(19) United States
(12) Patent Application Publication
(10) Pub. No.: US 2015/0196708 A1
(43) Pub. Date: Jul. 16, 2015

(54) SYSTEMS, METHODS AND COMPOSITIONS FOR IMPROVED TREATMENT OF ACIDOSIS

(71) Applicants: The Regents of the University of California, Oakland, CA (US); THE UNITED STATES GOVERNMENT REPRESENTED BY THE DEPARTMENT OF VETERANS AFFAIRS, Washington, DC (US)

(72) Inventors: Thomas G. Mason, Los Angeles, CA (US); Jeffrey A. Kraut, Santa Monica, CA (US)

(21) Appl. No.: 14/420,861

(22) PCT Filed: Aug. 19, 2013
(86) PCT No.: PCT/US2013/055629

§ 371 Date: Feb. 10, 2015

Related U.S. Application Data
(60) Provisional application No. 61/684,618, filed on Aug. 17, 2012.
(60) Provisional application No. 61/831,480, filed on Jun. 5, 2013.

(51) Int. Cl. A61M 5/14; A61K 33/00; A61K 9/19; A61M 5/172

(52) U.S. Cl. CPC A61M 5/1409; A61M 5/1723; A61K 33/00; A61K 9/19; A61M 2230/208; A61M 2230/202

(57) ABSTRACT

A system for treating an acidotic patient includes an intravenous-fluid supply system, an automated fluid mixer and dispenser connected to the intravenous-fluid supply system to receive at least one supply fluid therefrom, an electronic control system configured to communicate with the automated fluid mixer and dispenser, and an intravenous line fluidly connected to the automated fluid mixer and dispenser. The intravenous line includes an intravenous connecter configured for injecting intravenous fluid dispensed from the automated fluid mixer and dispenser intravenously into the acidotic patient. The electronic control system is configured to control at least one of a total volume or a flow rate of the intravenous fluid to be injected into the acidotic patient's blood based on at least a measured pH of the acidotic patient's blood and based on a composition of the at least one supply fluid. An intravenous solution for treating acidosis includes sodium bicarbonate and at least one of disodium carbonate, sodium hydroxide, and tris(hydroxymethyl)aminomethane dissolved in an aqueous solution. The intravenous solution has a pH of at least 10 and a total concentration of osmolites within a near isotonic range.

### CROSS-REFERENCE OF RELATED APPLICATION

This application claims priority to U.S. Provisional Application No. 61/684,618 filed Aug. 17, 2012; and U.S. Provisional Application No. 61/831,480 filed Jun. 5, 2013, the entire contents of which are hereby incorporated by reference.

### BACKGROUND

1. Field of Invention

The field of the currently claimed embodiments of this invention relates to systems, methods and compositions for improved treatment of acidosis.

2. Discussion of Related Art

Acute metabolic acidosis (a few hours to a few days in duration) and chronic metabolic acidosis (weeks to years in duration) are associated with impaired cellular function with increase morbidity and mortality (Kraut and Madias, 2010; Kraut, 2011). Presently, in addition to eliminating the underlying cause, treatment options are limited primarily to administration of base. Intravenous sodium bicarbonate has been the main base utilized in the treatment of acute metabolic acidosis. However, there is controversy about its use (Kraut and Kurtz, 2006). In human studies, administration of bicarbonate had no more beneficial effect than the administration of similar quantities of sodium chloride (Cooper et al., 1990). Moreover, the administration of base did not reduce morbidity or mortality of patients with ketoacidosis or lactic acidosis (Kraut and Madias, 2012). The failure of bicarbonate to be beneficial has been attributed to, in part, a reduction in intracellular pH arising from generation of carbon dioxide with its administration, the carbon dioxide rapidly penetrating cells producing an intracellular respiratory acidosis (Kraut and Madias, 2010). Therefore there is a need for development of a new base that can raise extracellular and intracellular pH and eliminate, or even reduce carbon dioxide levels in tissues.

Carbicarb, a 1:1 mixture of sodium dicarbonate and sodium bicarbonate (Filley and Kindig, 1985; Bersin and Arieff, 1988), does not increase generation of carbon dioxide while raising interstitial pH and maintaining or raising intracellular pH (Shapiro et al., 1995). Although early studies demonstrated improvement in cardiac function in animals, limited human studies involving 1:1 Carbicarb showed only minimal benefit (Leung et al., 1994).

Examination of the properties of the individual constituents of 1:1 Carbicarb, however, suggest that alteration of the ratio of dicarbonate to bicarbonate could improve the buffering capability and actually reduce tissue carbon dioxide levels (see Table 1).

TABLE 1
Impact of base administration on the pH and carbon dioxide
pressure PCO₂. (Shapiro et al., 1995)

| | pH | PCO₂ (mmHg) |
|---|---|---|
| Baseline (after HCl) | 6.81 ± .03 | 137 ± 10 |
| 0.5M NaHCO₃ | 6.92 ± 0.04 | 240 ± 6 |
| 0.5M Na₂CO₃ | 8.82 ± .08 | 3 ± 2 |
| 0.5M NaHCO₃/Na₂CO₃ | 7.35 ± .07 | 91 ± 9 |

The methods of delivery of the intravenous base could also affect the effectiveness of the administered buffer. The complications of bicarbonate administration have been shown to be more severe when the bicarbonate is administered rapidly as a hyperosmolal solution. Also, adverse effects at the infusion site were seen when there was extravasation of a highly alkaline solution. Therefore, development of methods to deliver the base slowly in a graded fashion could be helpful in eliminating these complications.

In summary, the optimum combination of compositions (i.e. materials and concentrations) and methods of introduction for treating various types of acidosis and degrees of acidosis (e.g. how far below normal body pH) have not yet been determined. An optimum combination for a particular type of acidosis would produce a desired increase in pH (in systemic blood as well as the interstitial and intracellular compartments) without causing adverse side-effects, particularly tissue damage near the administration or injection site (e.g. due to the pH of the injected solution being higher than normal body pH levels for an extended period of time and causing caustic tissue damage). Thus, there still remains an unmet need for formulating appropriate buffers (i.e. solutions) and/or administering them in a more efficacious manner which can improve upon the prior art of treating metabolic acidosis.

### SUMMARY

A system for treating an acidotic patient according to an embodiment of the current invention includes an intravenous-fluid supply system, an automated fluid mixer and dispenser connected to the intravenous-fluid supply system to receive at least one supply fluid therefrom, an electronic control system configured to communicate with the automated fluid mixer and dispenser, and an intravenous line fluidly connected to the automated fluid mixer and dispenser. The intravenous line includes an intravenous connecter configured for injecting intravenous fluid dispensed from the automated fluid mixer and dispenser intravenously into the acidotic patient. The electronic control system is configured to control at least one of a total volume or a flow rate of the intravenous fluid to be injected into the acidotic patient's blood based on at least a measured pH of the acidotic patient's blood and based on a composition of the at least one supply fluid.

An intravenous solution for treating acidosis according to an embodiment of the current invention includes sodium bicarbonate and at least one of disodium carbonate, sodium hydroxide, and tris(hydroxymethyl)aminomethane dissolved in an aqueous solution. The intravenous solution has a pH of at least 10 and a total concentration of osmolites within a near isotonic range.

A method of treating acidosis according to an embodiment of the current invention includes providing an intravenous solution for treating acidosis, and administering the intravenous solution intravenously to an acidotic patient. The intravenous solution has a pH of at least 10 and a total concentration of osmolites within a near isotonic range.

An intravenous dispersion for treating acidosis according to an embodiment of the current invention includes a liquid and a plurality of particles dispersed in the liquid. Each particle of the plurality of particles has a maximum outer dimension of less than about 2 micrometers such that the particles can pass unhindered through capillary blood vessels of an acidotic patient being treated. Each of the plurality of particles includes at least one of a shell and a matrix material that dissolves at a predetermined rate within the acidotic patient's blood stream, and each of the plurality of particles includes a pH-influencing material that mixes in the acidotic patient's blood stream at a controlled rate while the at least one of the shell and the matrix material dissolves.

### BRIEF DESCRIPTION OF THE DRAWINGS

Further objectives and advantages will become apparent from a consideration of the description, drawings, and examples.

FIG. 1 shows equilibrium pH of a solution having 0.5 M carbonate species formed by dissolving sodium bicarbonate and sodium carbonate in neutral water. The initial concentration of the bicarbonate anion immediately after dissolution and dissociation is [HCO₃⁻]₀. The initial concentration of the carbonate anion immediately after dissolution and dissociation is [CO₃²⁻]₀. The pH is plotted as a function of the ratio [HCO₃⁻]₀/[CO₃²⁻]₀, and the limiting values of pH at very low and very high ratio correspond to the pH of a pure sodium carbonate solution at 0.5 M and a pure sodium bicarbonate solution at 0.5 M, respectively.

FIG. 2 shows equilibrium pH of a solution having 0.01 M carbonate species formed by dissolving sodium bicarbonate and sodium carbonate in neutral water. The initial concentration of the bicarbonate anion immediately after dissolution and dissociation is [HCO₃⁻]₀. The initial concentration of the carbonate anion immediately after dissolution and dissociation is [CO₃²⁻]₀. The pH is plotted as a function of the ratio [HCO₃⁻]₀/[CO₃²⁻]₀, and the limiting values of pH at very low and very high ratio correspond to the pH of a pure sodium carbonate solution at 0.01 M and a pure sodium bicarbonate solution at 0.01 M, respectively.

FIG. 3 shows equilibrium pressure of CO₂, pCO₂, of a solution having 0.5 M carbonate species formed by dissolving sodium bicarbonate and sodium carbonate in neutral water plotted as a function of the ratio [HCO₃⁻]₀/[CO₃²⁻]₀.

FIG. 4 shows equilibrium pressure of CO₂, pCO₂, of a solution having 0.01 M carbonate species formed by dissolving sodium bicarbonate and sodium carbonate in neutral water plotted as a function of the ratio [HCO₃⁻]₀/[CO₃²]₀.

FIG. 5 is a schematic illustration of a system for treating an acidotic patient according to an embodiment of the current invention.

FIG. 6 is a schematic illustration of a system for treating an acidotic patient according to another embodiment of the current invention.

FIG. 7 is a schematic illustration of a system for treating an acidotic patient according to another embodiment of the current invention.

FIG. 8 is a schematic illustration of a system for treating an acidotic patient according to another embodiment of the current invention.

FIGS. 9A-9C are schematic illustrations of nanoparticles for forming dispersions for treating an acidotic patient according to some embodiments of the current invention.

FIG. 10 shows measured pH of a 0.50 M solution of sodium carbonate and sodium bicarbonate over a wide range of ratios given by the initial bicarbonate ion concentration divided by the initial carbonate ion concentration [HCO₃⁻]₀/[CO₃²⁻]₀.

FIG. 11 shows calculated pH of an aqueous solution of sodium bicarbonate and sodium hydroxide, where the initial concentration of sodium bicarbonate is fixed at [HCO₃⁻]₀=0.25 M, as a function of the initial concentration of sodium hydroxide (and therefore the hydroxide ion) [OH⁻]₀.

FIG. 12 shows calculated pCO₂ of an aqueous solution of sodium bicarbonate and sodium hydroxide, where the initial concentration of sodium bicarbonate is fixed at [HCO₃⁻]₀=0.25 M, as a function of the initial concentration of sodium hydroxide (and therefore the hydroxide ion) PHI. Units of pCO₂ are in mm Hg.

FIGS. 13A-13D provides details of the content of HydroxyBicarb calculations corresponding to FIGS. 11 and 12.

FIGS. 14A-14D show details of calculations corresponding to FIGS. 1-4.

FIG. 15 shows pH of canine blood of 7 May 2013, measured using an IDEXX Vetstat blood-gas analyzer (BGA), as a function of concentration of Na₂CO₃ base solution (1 part Na₂CO₃ solution added to 9 parts canine blood). Molarity of the base solution refers to molarity of carbonate species. The pH value at zero molarity corresponds to untreated canine blood. The addition of this base solution raises pH. A linear least squares fit (solid line) to the data yield an intercept of 6.99±0.03 pH units and a slope of 0.0037+0.0003 pH units/mM. Measurements (not shown) at 200 mM, 250 mM, and 360 mM gave results of pH>7.8, the upper limit of the BGA's measurement capability.

FIG. 16 provides PCO2 of canine blood of 7 May 2013, measured using an IDEXX Vetstat blood-gas analyzer (BGA), as a function of concentration of Na₂CO₃ base solution (1 part Na₂CO₃ solution added to 9 parts canine blood). Molarity of the base solution refers to molarity of carbonate species. The PCO2 value at zero molarity corresponds to untreated canine blood. The addition of this base solution lowers PCO2. A measurement (not shown) of PCO2 at 360 mM yielded a result of <10 mm Hg, below the measurement limit of the BGA of 10 mm Hg.

FIG. 17 shows equilibrium concentration of the bicarbonate ion, HCO₃⁻, in canine blood of 7 May 2013, measured using an IDEXX Vetstat blood-gas analyzer (BGA), as a function of concentration of Na₂CO₃ base solution (1 part Na₂CO₃ solution added to 9 parts canine blood). Molarity of the base solution refers to molarity of carbonate species. The [HCO₃⁻]_{eq} value at zero molarity corresponds to untreated canine blood. The addition of this base solution raises [HCO₃⁻]_{eq}.

FIG. 18 shows pH of treated canine blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species is thus fixed at 150 mM. One part of the treatment base solution is added to 9 parts of canine blood. The average increase in pH≈0.60 as a result of adding 150 mM strong-base treatment solution is nearly independent of the type of strong base added; this corresponds to a change of 0.004 pH units/mM strong base.

FIG. 19 shows PCO2 of treated canine blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species in the treatment base solution is thus fixed at 150 mM. One part of the treatment base solution is added to 9 parts of canine blood.

FIG. 20 shows equilibrium bicarbonate ion concentration of treated canine blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species in the treatment base solution is thus fixed at 150 mM. One part of the treatment base solution is added to 9 parts of canine blood.

FIG. 21 shows equilibrium sodium ion concentration of treated canine blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species in the treatment base solution is thus fixed at 150 mM. One part of the treatment base solution is added to 9 parts of canine blood.

FIG. 22 shows pH of base-treated canine HCl-acidified blood of 9 May 2013, measured using an IDEXX Vetstat blood-gas analyzer (BGA), as a function of concentration of Na₂CO₃ base solution (1 part Na₂CO₃ solution added to 9 parts canine HCl-acidified blood). Molarity of the base solution refers to molarity of carbonate species. The pH value at zero molarity corresponds to HCl-acidified canine blood. The addition of this base solution raises pH. A linear least squares fit (solid line) to the data yield an intercept of 6.62±0.03 pH units and a slope of 0.0050+0.0002 pH units/mM. The pH measurement at added [Na₂CO₃]=250 mM was made using a pH meter, not the BGA, since the pH lies above the measurement range of the BGA.

FIG. 23 shows PCO2 of base-treated canine HCl-acidified blood of 9 May 2013, measured using an IDEXX Vetstat blood-gas analyzer (BGA), as a function of concentration of Na₂CO₃ base solution (1 part Na₂CO₃ solution added to 9 parts canine HCl-acidified blood). Molarity of the base solution refers to molarity of carbonate species. The PCO2 value at zero molarity corresponds to canine HCl-acidified blood.

FIG. 24 shows equilibrium concentration of the bicarbonate ion in base-treated canine HCl-acidified blood of 9 May 2013, measured using an IDEXX Vetstat blood-gas analyzer (BGA), as a function of concentration of Na₂CO₃ base solution (1 part Na₂CO₃ solution added to 9 parts canine HCl-acidified blood). Molarity of the base solution refers to molarity of carbonate species. The [HCO₃⁻]_{eq} value at zero molarity corresponds to canine HCl-acidified blood.

FIG. 25 shows equilibrium concentration of the sodium ion in base-treated canine HCl-acidified blood of 9 May 2013, measured using an IDEXX Vetstat blood-gas analyzer (BGA), as a function of concentration of Na₂CO₃ base solution (1 part Na₂CO₃ solution added to 9 parts canine HCl-acidified blood). Molarity of the base solution refers to molarity of carbonate species. The [Na⁺]_{eq} value at zero molarity corresponds to canine HCl-acidified blood.

FIG. 26 shows pH of base-treated canine HCl-acidified blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species is thus fixed at 150 mM. One part of the treatment base solution is added to 9 parts of canine HCl-acidified blood. The average increase in pH≈0.68 as a result of adding 150 mM strong-base treatment solution is nearly independent of the type of strong base added; this corresponds to a change of 0.0045 pH units/mM strong base.

FIG. 27 shows PCO2 of base-treated canine HCl-acidified blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species in the treatment base solution is thus fixed at 150 mM. One part of the treatment base solution is added to 9 parts of canine HCl-acidified blood.

FIG. 28 shows equilibrium bicarbonate ion concentration of base-treated canine HCl-acidified blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species in the treatment base solution is thus fixed at 150 mM. One part of the treatment base solution is added to 9 parts of canine HCl-acidified blood.

FIG. 29 shows equilibrium sodium ion concentration of base-treated canine HCl-acidified blood of 9 May 2013, measured using an IDEXX Vetstat blood-gas analyzer (BGA), as a function of percentage of NaOH in a treatment base solution that is a mixture of sodium hydroxide and disodium carbonate. The points shown correspond to near-isotonic treatment base solutions of 150 mM NaOH (100%), 150 mM Na₂CO₃ (0%), and 1 part 150 mM Na₂CO₃ solution mixed with 1 part 150 mM NaOH (50%). The concentration of strong-base species in the treatment base solution is thus fixed at 150 mM. One part of the treatment base solution is added to 9 parts of canine HCl-acidified blood.

FIG. 30 shows pH of base-treated canine HCl-acidified blood of 9 May 2013, measured using an IDEXX Vetstat blood-gas analyzer (BGA), as a function of total carbonate concentration of base-treatment solution consisting of an equimolar solution of sodium bicarbonate and disodium carbonate. The base-treatment solution at 667 mM total carbonate concentration is classic “carbicarb” (333 mM NaHCO₃+333 mM Na₂CO₃). One part base-treatment solution is added to 9 parts canine HCl-acidified blood. The pH value at zero molarity corresponds to that of canine HCl-acidified blood. The addition of this base solution raises pH. A linear least squares fit (solid line) to the data yield an intercept of 6.71±0.20 pH units and a slope of 0.0026+0.0005 pH units/mM. The pH measurement at a total carbonate concentration of 667 mM in the base-treatment solution was made using a pH meter, not the BGA, since the pH lies above the measurement range of the BGA.

FIG. 31 shows PCO2 of base-treated canine HCl-acidified blood of 9 May 2013, measured using an IDEXX Vetstat blood-gas analyzer (BGA), as a function of total carbonate concentration of base-treatment solution consisting of an equimolar solution of sodium bicarbonate and disodium carbonate. The base-treatment solution at 667 mM total carbonate concentration is classic “carbicarb” (333 mM NaHCO₃+333 mM Na₂CO₃). One part base-treatment solution is added to 9 parts canine HCl-acidified blood. The PCO2 value at zero molarity corresponds to that of canine HCl-acidified blood.

FIG. 32 shows equilibrium bicarbonate ion concentration of base-treated canine HCl-acidified blood of 9 May 2013, measured using an IDEXX Vetstat blood-gas analyzer (BGA), as a function of total carbonate concentration of base-treatment solution consisting of an equimolar solution of sodium bicarbonate and disodium carbonate. The base-treatment solution at 667 mM total carbonate concentration is classic “carbicarb” (333 mM NaHCO₃+333 mM Na₂CO₃). One part base-treatment solution is added to 9 parts canine HCl-acidified blood. The [HCO₃⁻]_{eq} value at zero molarity corresponds to that of canine HCl-acidified blood.

FIG. 33 shows equilibrium sodium ion concentration of base-treated canine HCl-acidified blood of 9 May 2013, measured using an IDEXX Vetstat blood-gas analyzer (BGA), as a function of total carbonate concentration of base-treatment solution consisting of an equimolar solution of sodium bicarbonate and disodium carbonate. The base-treatment solution at 667 mM total carbonate concentration is classic “carbicarb” (333 mM NaHCO₃+333 mM Na₂CO₃). One part base-treatment solution is added to 9 parts canine HCl-acidified blood. The [Na⁺]_{eq} value at zero molarity corresponds to that of canine HCl-acidified blood.

FIG. 34 shows pH of base-treated canine HCl-acidified blood of 21 May 2013, measured using an IDEXX Vetstat blood-gas analyzer (BGA), as a function of percent disodium carbonate in a mixed base-treatment solution of sodium bicarbonate and disodium carbonate, where the total concentration of added carbonate species of the base-treatment solution is fixed at 150 mM. One part base-treatment solution is added to 9 parts canine HCl-acidified blood. The dashed line shows the pH of HCl-acidified canine blood of 21 May 2013 prior to treatment with the base-treatment solution.

FIG. 35 shows PCO2 of base-treated canine HCl-acidified blood of 21 May 2013, measured using an IDEXX Vetstat blood-gas analyzer (BGA), as a function of percent disodium carbonate in a mixed base-treatment solution of sodium bicarbonate and disodium carbonate, where the total concentration of added carbonate species of the base-treatment solution is fixed at 150 mM. One part base-treatment solution is added to 9 parts canine HCl-acidified blood of 21 May 2013. The dashed line shows the PCO2 of HCl-acidified canine blood of 21 May 2013 prior to treatment with the base-treatment solution.

FIG. 36 shows equilibrium concentration of bicarbonate ion [HCO₃⁻]_{eq} in base-treated canine HCl-acidified blood of 21 May 2013, measured using an IDEXX Vetstat blood-gas analyzer (BGA), as a function of percent disodium carbonate in a mixed base-treatment solution of sodium bicarbonate and disodium carbonate, where the total concentration of added carbonate species of the base-treatment solution is fixed at 150 mM. One part base-treatment solution is added to 9 parts canine HCl-acidified blood of 21 May 2013. The dashed line shows the [HCO₃⁻]_{eq} of HCl-acidified canine blood of 21 May 2013 prior to treatment with the base-treatment solution.

FIG. 37 shows equilibrium concentration of sodium ion [Na⁺]_{eq} in base-treated canine HCl-acidified blood of 21 May 2013, measured using an IDEXX Vetstat blood-gas analyzer (BGA), as a function of percent disodium carbonate in a mixed base-treatment solution of sodium bicarbonate and disodium carbonate, where the total concentration of added carbonate species of the base-treatment solution is fixed at 150 mM. One part base-treatment solution is added to 9 parts canine HCl-acidified blood of 21 May 2013. The dashed line shows the [Na⁺]_{eq} of HCl-acidified canine blood of 21 May 2013 prior to treatment with the base-treatment solution.

FIG. 38 shows brightfield transmission optical micrograph of untreated canine blood of 9 May 2013. Nearly all red blood cells (RBCs) are biconcave (i.e. normal) in shape and undamaged (few RBCs are spiculated). Rouleaux (i.e. attractive columnar aggregates of RBCs) are not observed. Scale: the field of view shown is about 298 microns×184 microns.

FIG. 39 shows brightfield optical transmission micrograph of canine blood of 9 May 2013 treated with 1 M sodium bicarbonate solution (1 part 1 M NaHCO₃ solution added to 9 parts blood). Many RBCs have been damaged by the treatment; many fragments of RBCs (i.e. schistocytes) and highly deformed RBCs are observed. Scale: the field of view shown is about 298 microns×184 microns.

FIG. 40 shows brightfield transmission optical micrograph of canine blood of 9 May 2013 treated with a near-isotonic solution of disodium carbonate (1 part 150 mM aqueous solution of Na₂CO₃ added to 9 parts blood). Nearly all red blood cells (RBCs) are biconcave (i.e. normal) in shape and undamaged (few RBCs are spiculated). Rouleaux (i.e. attractive columnar aggregates of RBCs) are not observed. Scale: the field of view shown is about 298 microns×184 microns.

FIG. 41 shows brightfield transmission optical micrograph of canine blood of 9 May 2013 treated with a near-isotonic solution of sodium hydroxide (1 part 150 mM aqueous solution of NaOH added to 9 parts blood). Nearly all red blood cells (RBCs) are biconcave (i.e. normal) in shape but a minor fraction of RBCs are spiculated. However, rouleaux (i.e. attractive columnar aggregates of RBCs) are frequently observed. Scale: the field of view shown is about 298 microns×184 microns.

FIG. 42 shows brightfield transmission optical micrograph of canine blood of 9 May 2013 treated with a near-isotonic solution 75 mM sodium hydroxide: 75 mM disodium carbonate (1 part 150 mM strong base aqueous solution added to 9 parts blood). Nearly all red blood cells (RBCs) are biconcave (i.e. normal) in shape but a minor fraction of RBCs are spiculated. Some rouleaux (i.e. attractive columnar aggregates of RBCs) are observed: more than in FIG. 40 but far fewer than in FIG. 41. Scale: the field of view shown is about 298 microns×184 microns.

FIG. 43 shows brightfield transmission optical micrograph of canine blood of 9 May 2013 treated with 150 mM HCl solution to acidify the blood (1 part 150 mM aqueous solution of HCl added to 9 parts blood). A large fraction of RBCs are spiculated, compared to the untreated blood in FIG. 38. Rouleaux (i.e. attractive columnar aggregates of RBCs) of non-spiculated RBCs are also frequently observed. Significant irreversible damage to RBCs, primarily observed as spiculation, by HCl-acidification of the blood can complicate the interpretation of micrographs of HCl-acidified blood that is subsequently treated by base solutions. Scale: the field of view shown is about 298 microns×184 microns.

FIG. 44 shows brightfield transmission optical micrograph of canine blood of 9 May 2013 treated with equimolar carbonate solution of sodium bicarbonate at 333 mM plus disodium carbonate at 333 mM (1 part 333 mM NaHCO₃+333 mM Na₂CO₃ aqueous solution added to 9 parts blood). A large fraction of RBCs exhibit spiculation, deformation, or breakage; a significant fraction of schistocytes are also observed compared to the untreated blood in FIG. 38. Some rouleaux (i.e. attractive columnar aggregates of RBCs) are also observed. Scale: the field of view shown is about 298 microns×184 microns.

FIG. 45 shows brightfield transmission optical micrograph of canine blood of 21 May 2013 treated with a near-isotonic solution of saline-supplemented sodium hydroxide (1 part 150 mM aqueous solution of NaOH containing 75 mM NaCl added to 9 parts blood). Nearly all red blood cells (RBCs) are biconcave (i.e. normal) in shape and very few RBCs are spiculated. However, a few smaller rouleaux are present. Overall, the addition of a small saline concentration appears to reduce the amount of spiculation and rouleaux formation compared to 150 mM NaOH only treatment shown in FIG. 41. Scale: the field of view shown is about 298 microns×184 microns.

FIG. 46 shows measured pH during titration of an initial volume of 4 mL of HCl-acidified canine blood using an added volume V_{b} of a near-isotonic base-treatment solution: 150 mM NaHCO₃ (squares), 100 mM Na₂CO₃ (circles), and 100 mM NaOH: 100 mM NaCl (triangles). The titration has been performed at 23° C.

FIG. 47 shows an embodiment of a computer-controlled system and custom-written computer software for dispensing liquid base-treatment solutions, including the option of feedback control based on signals of blood pH measured by a pH measuring device. As configured, the liquid dispensing device has two separate 25 mL syringes mounted in a computer-controllable dual-syringe Hamilton Microlab 560 syringe pump. These syringes are connected to two separate computer-controlled valves that enable the syringes to be refilled from separate liquid reservoirs (here shown in two different beakers), which hold two liquid solutions. Two separate input polymer-tubing lines are arranged to transport liquids from the two liquid reservoirs to the two computer-controlled valves. The volume rate of liquid dispensed and total volume of liquid dispensed from each syringe can be independently controlled, and both syringes can dispense liquids simultaneously at different rates via two output tubing lines. A pH meter (Accumet AB150) and pH probe are connected to the control computer (Dell Precision 490 workstation). As shown here, there are two output polymer-tubing lines that are connected to the top of the two separate computer-controlled valves. The ends of these tubing lines are connected to a patient's circulatory system through needles at two separate points on the patient. The computer software continuously reads the pH from the pH meter's probe (which can be contacted with the blood of the patient via a probe mounted in a perforated needle that is inserted into the patient's circulatory system) through a serial digital electronic interface with the pH meter, and the computer software is programmed to alter or stop the flow of liquids based on the real-time pH measurement and stored information and equations that are part of the computer software program.

FIG. 48 shows total circulating blood volume (blue squares), remaining original blood volume (red circles), injected 0.55 M lactic acid solution volume (green diamonds), and volume of near-isotonic base solution 112.5 mM Na₂CO₃+37.5 mM NaHCO₃ injected (black triangles) as a function of time after the initial bleed for RAT A. RAT A characteristics: male Sprague-Dawley, weight 246.6 g, date of birth Apr. 9, 2013. The 0.55 M lactic acid solution is injected into the femoral vein using a programmable syringe pump, and stopped. Subsequently, the base solution is injected into the femoral vein using a programmable syringe pump. The total injected volume of base solution is about 3.3 mL.

FIG. 49 shows blood-gas parameters pH (upper panel), PCO2 (middle panel) and [HCO3-] (lower panel) of RAT A measured using a blood-gas analyzer (IDEXX VetStat) as a function of time after the initial bleed, corresponding to the injected volumes of acid and base solutions described in FIG. 1. Treatment of the acidotic state of RAT A (at about 90 min) by about 3.3 mL of the near-isotonic base solution (112.5 mM Na₂CO₃+37.5 mM NaHCO₃) results in: an increase in pH (averaged over arterial and venous), ΔpH=+0.26; a very small average change in pressure of CO₂, ΔPCO2=+2.5 mm Hg; and an average increase in bicarbonate ion concentration, Δ[HCO3⁻]+9.4 mM.

FIG. 50 shows blood ion concentrations [Na⁺] and [Cl⁻] (upper panel) and [K⁺] (lower panel) of RAT A measured using a blood-gas analyzer (IDEXX VetStat) as a function of time after the initial bleed, corresponding to the injected volumes of acid and base solutions described in FIG. 1. These ion concentrations in the blood of RAT A are not strongly affected by the injected solutions.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some embodiments of the current invention can provide an improved form of base and base delivery for treatment of Acute Metabolic Acidosis. The following describes new systems, new materials (e.g. forms of bases) and new methods of base administration that can enable more efficacious treatment of acidosis, such as, but not limited to acute metabolic acidosis, while minimizing or preventing undesirable side-effects.

### New Form of Base for Treatment of Acute Metabolic Acidosis

Utilization of bicarbonate-based solutions, rather than alternative buffers such as THAM, is attractive because in an open system, as present in the body, carbon dioxide produced in the buffering process can be eliminated through the lungs. By contrast, THAM with accompanying protons has to be excreted in the urine. On the other hand if the buffering can be accomplished while minimizing the carbon dioxide produced, this can provide a distinct advantage. The optimal ratio of NaHCO₃:Na₂CO₃ dissolved to form an aqueous solution for intravenous administration in the treating of acute metabolic acidosis has never been determined for different types and degrees of acidosis. The choice of a 1:1 ratio for the Carbicarb formulation in the past was presumed because its administration did not increase generation of carbon dioxide and also raised extracellular and intracellular pH in animal studies (Bersin and Arieff, 1988). However, as indicated in Table 1, dicarbonate per se actually consumes carbon dioxide, a valuable property which enhances its value as a buffer since increased carbon dioxide tissue levels often accompany states associated with lactic acidosis. Therefore, generation of a base formulation having a greater dicarbonate to bicarbonate ratio would be desirable. Herein, we use bicarbonate to refer to HCO₃⁻, and we use either dicarbonate or carbonate to refer to CO₃²⁻.

The choice of a 1:1 ratio for the Carbicarb formulation used in the past, in combination with a standard injection scenario, is too caustic for injection and administration by the standard method of treatment, leading to tissue damage in the patient near the injection site resulting from prolonged exposure of this tissue to a significantly higher pH than is normal. Such tissue damage local to the injection site has been reported for concentrated solutions of sodium carbonate (see e.g. the Medical History section of Filley and Kindig 1985), which are significantly more basic (pH typically above 10) than concentrated solutions of sodium bicarbonate (pH typically near 8.3).

Although a sodium bicarbonate solution could be a good starting point for an improved treatment of acidosis in terms of known safe use in humans, sodium carbonate is not the only alternative material that could be used to raise the pH of the treatment solution. An alternative treatment solution for acidosis that has a higher pH than that of a sodium bicarbonate solution can be more efficacious in terms of raising a patient's overall pH without creating unwanted side-effects; however, sustained exposure of the patient to a higher pH of an alternative treatment solution could potentially cause unwanted tissue damage, especially near the injection site where the treatment is administered to the patient. For instance, sodium hydroxide is an example of a strong base that, when added in low concentrations judiciously, could be used in conjunction with a buffer like sodium bicarbonate to raise the pH of the alternative treatment solution above that of pure sodium bicarbonate solution. In fact, as is pointed out in Oxtoby et al., addition of a strong base to the amphoteric carbonate system in water can lead to an increase in pH and a change in the ratios of the equilibrium concentrations of bicarbonate ions and carbonate ions.

Using equilibrium equations of acid-base physical chemistry, it is possible to solve for the equilibrium pH of an aqueous solution resulting by dissolving into pure neutral water an initial quantity of sodium bicarbonate, yielding an initial bicarbonate anion concentration [HCO₃⁻]₀ and an initial quantity of sodium carbonate, yielding an initial carbonate anion concentration [CO₃²⁻]₀ immediately following dissolution and dissociation. With these initial concentrations, using equations for water autoionization, charge neutrality, conservation of matter (i.e. carbonate species), law of mass action for the deprotonation of carbonic acid H₂CO₃ in water into a bicarbonate anion and a hydronium ion, and law of mass action for the deprotonation of the bicarbonate anion in water into a carbonate anion and a hydronium ion. The unknown concentrations at equilibrium are [H₃O⁺], [OH⁻], [Na⁺], [H₂CO₃], [HCO₃⁻], and [CO₃²⁻]. These equations can be reduced to a 4th order polynomial (i.e. quartic equation) in the hydronium ion concentration [H₃O⁺] (see below for details about the calculations). Quartic equations can be solved, and the solution of this equation is facilitated by use of the software program Mathematica. Out of four possible complex solutions, only the real, positive solution is physical, so it is chosen.

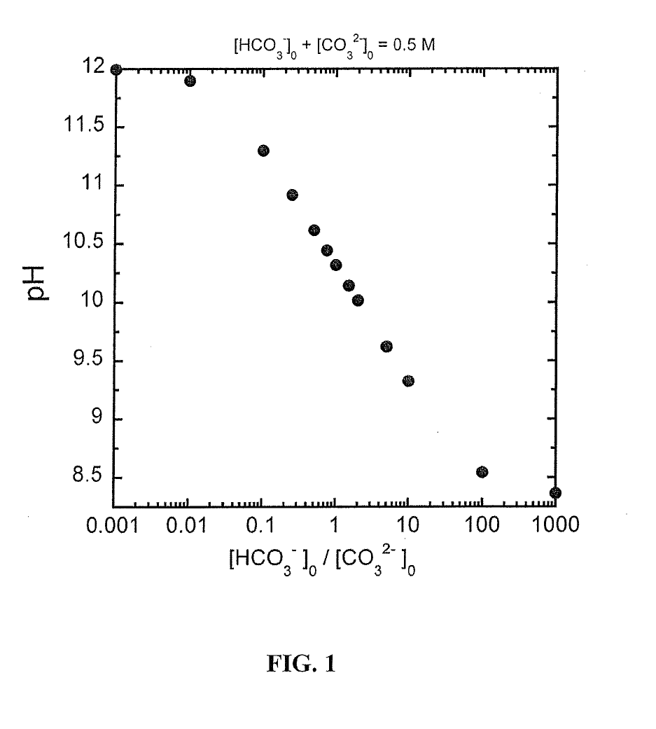

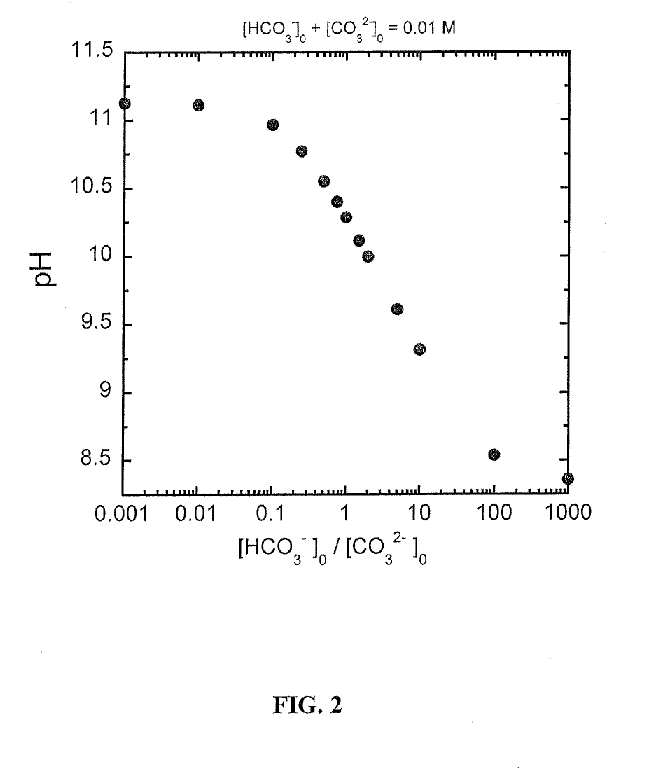

As an example, the predicted equilibrium pH at 0.5 M total carbonate concentration for different ratios of initial bicarbonate to carbonate concentrations is shown in FIG. 1. The result for the pH in the limit of large ratio (corresponding to pure bicarbonate solution) of around 8.34 is correct, according to a textbook solution of the pH of a sodium bicarbonate solution (see e.g. Oxtoby et al.). Values of Ka1=4.3×10⁻⁷ for carbonic acid deprotonation and Ka2=4.8×10⁻¹¹ for deprotonation of the bicarbonate anion are at room temperature, rather than body temperature, but that small change in temperature would make only a slight difference in the Ka1 and Ka2 values and thus the predicted pH. Note: the value of 4.3×10⁻⁷ chosen for Ka1 effectively incorporates the equilibrium between dissolved CO₂(aq) and H₂CO₃(aq). In the calculations, we designate the total carbonate concentration to be Cₜₒₜ=[HCO₃⁻]₀+[CO₃²⁻]₀ and we designate the initial ratio of bicarbonate to carbonate ions to be R=[HCO₃⁻]₀/[CO₃²⁻]₀.

From FIGS. 1 and 2, it can be seen that the predicted pH for 1:1 Carbicarb solution (where [HCO₃⁻]₀/[CO₃²⁻]₀=1) is about 10.2 and depends only very weakly on the overall concentration of carbonate species in a concentration range typical of a treatment solution. Thus, 1:1 Carbicarb's pH of about 10.2 is quite basic and caustic, and the effect of caustic tissue damage during the typical intravenous administration protocol is likely to be the reason why 1:1 Carbicarb has not been pursued commercially. However, this does not rule out custom-design of a solution that may contain a more optimal ratio of initial sodium bicarbonate to sodium carbonate concentration.

Although the trends are suggestive and can be used as a guideline for design purposes, these predictions for pH in FIGS. 1 and 2 are nevertheless somewhat approximate and further refinement may be necessary to obtain exact values that would match with experiment. So, the trend of the pH from a solution of pure sodium bicarbonate to a solution of pure sodium carbonate (e.g. as shown in FIGS. 1 and 2) is only approximate, and the actual measured equilibrium pH could differ somewhat from the values plotted. Thus, a measured pH curve as a function of overall carbonate concentration and [HCO₃⁻]₀/[CO₃²⁻]₀ could also be used for designing an optimal solution for treating acidosis.

Adding sodium carbonate (or other bases) in addition to sodium bicarbonate to neutral water can be used to raise the overall solution pH after equilibrium is reached, compared to the pH of a pure sodium bicarbonate solution. A higher pH of a treatment solution could be desirable from the point of view of raising overall blood pH when administered to a patient suffering from acidosis, provided undesirable side-effects, such as caustic damage near the injection site, can be limited to an acceptable level.

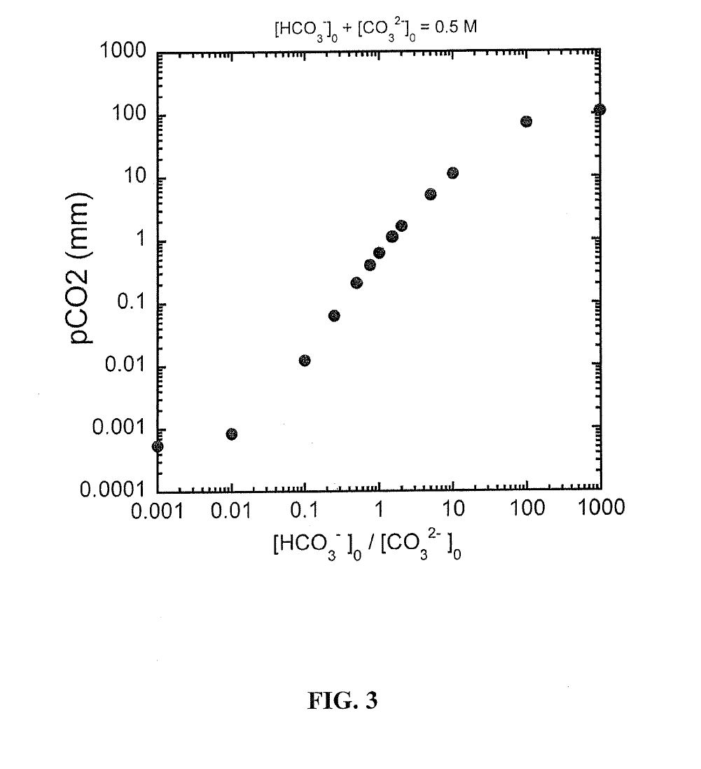

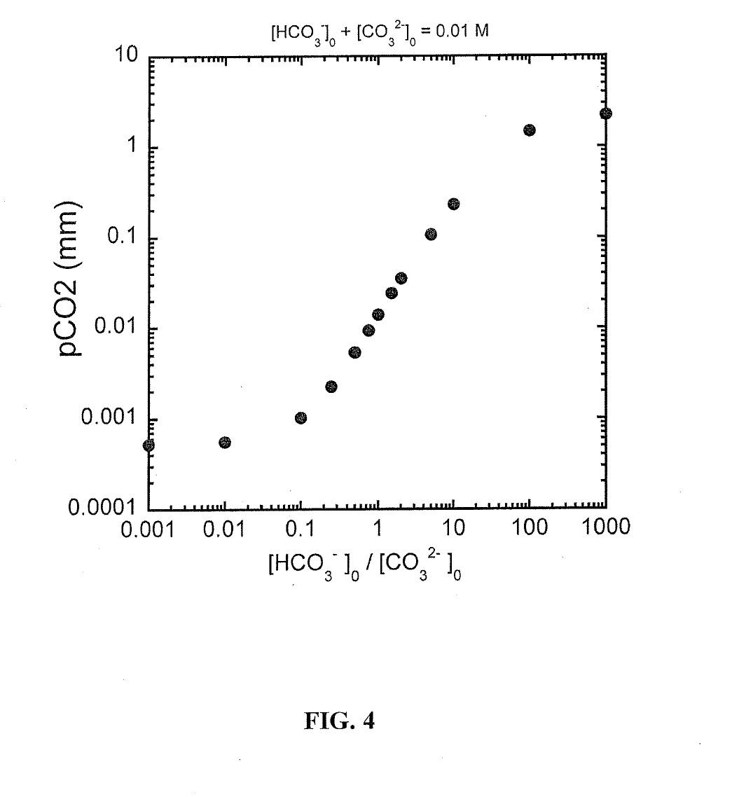

In addition to pH, the pressure of CO₂, pCO₂, in the solution of sodium carbonate and sodium bicarbonate to be administered is also important, since lower pCO₂ is typically desirable in formulations for treating acidosis. (pCO₂ may also be indicated PCO₂ herein. They are intended to have the same meaning.) Using the equilibrium equations, the actual concentration of CO₂ and pCO₂ of sodium carbonate and sodium bicarbonate solutions having different overall concentrations and ratios R=[HCO₃⁻]₀/[CO₃²⁻]₀ are predicted (see below for details). In FIG. 3, the result for Cₜₒₜ=0.5 M total carbonate concentration is shown, and in FIG. 4, the result for Cₜₒₜ=0.01 M total carbonate concentration is shown. For both total concentrations, a smaller ratio R, corresponding to more sodium carbonate in the formulation, reduces pCO₂.

In an embodiment of the current invention, the predicted equilibrium pH and pCO₂ as a function of total carbonate concentration Cₜₒₜ and R are used to select a treatment solution of a mixture of a solution of sodium carbonate and a solution of sodium bicarbonate to treat a patient suffering from acidosis based on at least a pH measurement of a patient.

In an embodiment of the current invention, a treatment solution consisting of a mixture of a solution of sodium carbonate and a solution of sodium bicarbonate having Cₜₒₜ>0.01 M and a ratio R>1 is administered to a patient suffering from acidosis.

Here, we use the common word “intravenous”, but administration of the treatment solution could be intra-arterial or via some other access route into the circulatory system and blood stream of the patient.

### Example Treatment Solution 1

Using the calculations shown in FIG. 1, a ratio [HCO₃⁻]₀/[CO₃²⁻]₀=30 at 0.5 M of a treatment solution (made from sodium bicarbonate and sodium carbonate) would provide a higher pH of about 8.8 (as compared to a solution of pure sodium bicarbonate). This solution having a higher pH than a pure sodium bicarbonate solution would be most appropriate for treating extreme acidosis, in which the patient needs to have blood and cellular pH raised substantially and more rapidly towards a normal level.

### Example Treatment Solution 2

Using the calculations shown in FIG. 1, a ratio [HCO₃⁻]₀/[CO₃²]₀=60 at 0.5 M of a treatment solution (made from sodium bicarbonate and sodium carbonate) would provide a higher pH of about 8.6 (as compared to a solution of pure sodium bicarbonate). This solution having a higher pH than a pure sodium bicarbonate solution would be most appropriate for treating mild acidosis, in which the patient needs to have blood and cellular pH raised mildly and gradually towards a normal level.

### Example Treatment Solution 3

The ratio of sodium bicarbonate to sodium carbonate mixed into water to form the treatment solution is determined according to the patient's measured blood pH relative to normal blood pH. If the patient's measured blood pH is in the extremely acidemic range (e.g. a pH below about 7.1), then the ratio [HCO₃⁻]₀/[CO₃²⁻]₀ is chosen to be below about 50, and if the patient's measured blood pH is in the mildly acidemic range (e.g. a pH between about 7.2 and about 7.3), then the ratio is chosen to be above about 50. This is assuming administration by standard intravenous delivery.

### Example Treatment Solution 4

In this example, a solution of sodium bicarbonate is mixed with a solution of sodium hydroxide, a different strong base than sodium carbonate. The addition of sodium hydroxide creates much the same effect as adding sodium carbonate in raising the solution's pH, compared to a solution of pure sodium bicarbonate, but yet does not contribute to the total carbonate concentration. For instance, a 0.5 M solution of sodium bicarbonate is added to a 0.1 M solution of sodium hydroxide to form a basic treatment solution suitable for treating acidosis.

### Example Treatment Solution 5

In this example, a solution of sodium bicarbonate is mixed with a solution of sodium carbonate to create a treatment solution having Cₜₒₜ=0.5 M and R in a range given by FIG. 3 that is selected such that pCO2 of the resulting solution is less than 10 mm.

### Example Treatment Method

Typical injection and treatment scenarios for acidosis typically administer a sodium bicarbonate solution through a standard intravenous injection at a single injection site into the patient's bloodstream. In this form of administration of the treatment solution, the rate of injection into the bloodstream over the course of administration is approximately constant, and it may even be unregulated. There is typically no feedback information used to actively control the composition, concentration, and/or rate of injection of a treatment solution. Typically, tissue in the region of the single injection site is subjected to a higher caustic exposure over a prolonged period of time than tissue further downstream and away from the injection site as a result of the caustic nature of the treatment solution prior to intermixing with the flowing blood. Whereas sodium bicarbonate solutions having a pH in the range of about 8.0 to 8.3 can be administered safely using this protocol, by contrast, other treatment solutions for acidosis, which may be designed to have higher pH to better raise blood pH, can create an adverse side-effect of undesirable tissue damage near, around, and downstream from the single injection site. So, there remains a need for improvements in combining the design of the administration method of a treatment solution for acidosis with the design of the composition of a treatment solution.

In this aspect of the current invention, a treatment solution having a higher pH than sodium bicarbonate solution is administered to a patient in a time-varying and spatially-varying manner that more evenly distributes the caustic exposure around a larger region of the body, thereby reducing and/or eliminating tissue damage that would otherwise occur if the treatment solution were injected using a standard method in a single injection site. Thus, regions of tissue proximate to the injection sites (where the intravenous (IV) needle is inserted into the patient) will have at least some time to recover closer to the body's normal pH (thereby avoiding or reducing tissue damage).

Accordingly, an embodiment of the current invention is to inject the treatment solution into the patient in a time-varying, controlled manner that enables the circulatory system of the patient to distribute the acid-neutralizing components in the treatment solution more evenly, thereby reducing the side-effect of local caustic tissue damage to an acceptable level.

In particular, in one embodiment, the volume rate of delivery of treatment solution is not constant, but can be varied in time (e.g. can be periodic in time yielding repeating cycles of a higher rate and a lower rate) so that tissue in the injection region exposed to the treatment solution has time to recover to closer to normal pH as the blood circulates and carries and distributes the treatment solution downstream in the patient.

Alternatively, in another embodiment, the pH of the treatment solution can also be varied in time (e.g. can be periodic in time yielding repeating cycles of a higher concentration and a lower concentration of the agents in the treatment solution) so that tissue in the injection region exposed to the treatment solution has time to recover to closer to normal pH as the blood circulates and carries and distributes the treatment solution downstream in the patient.

Alternatively, in yet another embodiment, the pH of the treatment solution can be varied in time in a non-periodic manner, starting at a higher pH and slowly decreasing to a lower pH. This can be accomplished, for instance, by changing the relative rates of a solution of sodium bicarbonate and a solution of sodium carbonate that are mixed together by a computer controlled system according to an embodiment of the current invention prior to injection into the patient. For example, the ratio [HCO₃⁻]₀/[CO₃²⁻]₀ can be selected as 10 initially (some sodium carbonate solution mixed with a sodium bicarbonate solution), and a quadratic increase in the ratio can be made over a two hour period to a final ratio of 1000 (nearly all sodium bicarbonate solution).

Alternatively, in yet another embodiment, the pH of the treatment solution can be varied in time in a non-periodic manner, starting at a lower pH and slowly increasing to a higher pH. This can be accomplished, for instance, by changing the relative rates of a solution of sodium bicarbonate and a solution of sodium carbonate that are mixed together by the computer controlled system prior to injection into the patient. For example, the ratio [HCO₃⁻]₀/[CO₃²⁻]₀ can be selected as 1000 initially (i.e. nearly all sodium bicarbonate solution), and an exponential decrease in the ratio can be made over a two hour period to a final ratio of 10 (i.e. some sodium carbonate solution mixed with a sodium bicarbonate solution).

A computer-controlled injection system according to an embodiment of the current invention having multiple solution containers, dispensers, valves, and mixers (e.g. multiple syringe pump dispensing/mixing systems such as manufactured by Hamilton or other companies) can be used to create the time-varying distribution of the agents in the treatment solution to the patient.

Another embodiment of the current invention that can better distribute the caustic load more evenly in the patient is to use multiple injection sites rather than a single injection site, so as to reduce or eliminate local tissue damage that may result from prolonged exposure of this tissue to a more highly caustic solution over a sustained period of time required to administer this solution. A different time-varying injection rate can be chosen for each different injection site. The time-varying composition, concentration, and volume rates at different injection sites can be independently controlled and they can be coordinated and/or synchronized. For instance, a computer-controlled mixer and dispenser that has computer controlled valves can alternate open and closed positions of valves periodically to cause the treatment solution to be injected into a first injection site for a certain period of time, then the flow to the first injection site is halted, and by action of computer-controlled valves, the treatment solution is then directed to and injected into a second injection site for a certain second period of time; then the computer-controlled system stops flow to the second injection site and redirects the flow of the treatment solution to the first injection site. The choice of the first period of time and second period of time can be determined in part by the diameters of the blood vessels and/or volume flow rates of blood in the blood vessels of the circulatory system of the patient at the first injection site and at the second injection site.

While we recognize that the use of multiple injection sites could create additional punctures to the circulatory system of the patient, which can be undesirable in certain circumstances, in some extreme cases, the positive benefit of distributing the caustic treatment solution more evenly throughout the patient can outweigh the negative side-effects caused by additional punctures to the patient's circulatory system. Embodiments of the current invention described herein that mention the use of multiple injection sites are only examples, and these embodiments could be applied as well to good effect in the use of only a single injection site.

In another embodiment of the current invention, a single needle for an IV having multiple side perforations and multiple fluid inputs (e.g. via two or more tubes) is inserted into the circulatory system of a patient, wherein fluid emanating from a certain first set of perforations are fed by a first tube and fluid emanating from a certain second set of perforations are fed by a second tube, so that the perforations spatially distribute the caustic solution out of different openings in the needle, over a greater spatial region than a needle having a single opening, in a time-varying manner, as a computer-controlled dispensing system changes the rates of fluid injection in said first tube and said second tube, thereby reducing tissue damage in the patient in the region proximate to said needle.

The manner in which the treatment solution is distributed to the multiple injection sites can be altered on-the-fly through real-time feedback control, wherein a measurement of a patient's pH is used as a factor in controlling the composition, concentration, and time-varying rate of injection of a treatment solution. In addition to a measurement of the patient's pH, other measurable aspects of a patient's condition (e.g. heart rate, blood pressure, temperature, CO₂ level in the blood, sodium concentration in the blood, and other factors) can be communicated to the computer-controlled dispensing system of a time-varying treatment solution. This communication between the sensors of the patient's current condition and history of condition and the control computer of the dispensing system of the treatment solution can occur by either wired digital, wired analog, wireless digital, and/or wireless analog communication, for example.

The above-described time-varying and space-varying method of administration of a treatment solution can offer significant advantages over the current simple method of constant IV injection, which causes tissue local to the injection site to experience an abnormally high pH for a long period of time while the a basic buffer is being administered (such a long period of higher pH is known to lead to considerable tissue damage locally around the injection site). A computer-controlled delivery system can optimize, in real-time, the rate of administration, pulsing, and relative amounts of mixtures of different solutions or buffers, based on how much of a change in pH the patient needs (i.e. patient's initial symptoms/pH and patient's current measured symptoms/pH).

For instance, in an embodiment of the present invention, a computer-controlled mixing and dispensing system could even vary the ratio of sodium carbonate solution to sodium bicarbonate solution administered to the patient via IV using a computer-controlled dispensers and valves as the patient's blood pH is changing over time (e.g. as monitored by a computer-linked sensor in a site on the patient different than the injection site(s)). This can include a computer-controlled feedback system for treating acidosis that could alter the ratio and concentrations of sodium carbonate to sodium bicarbonate over time, as the patient's pH approaches the normal range during the course of treatment, as well as providing pulsing, alternation, and/or redistribution of the solution to different IV injections points into the patient.

In an embodiment of the present invention, when a patient is just beginning treatment, the proportion of sodium carbonate in the composition of the treatment solution could be adjusted by the computer-controlled system to be somewhat higher temporarily, but as the patient's pH rises towards a more normal level, the computer could automatically reduce the proportion of sodium carbonate in the mixture being administered by IV as well as the overall concentration of the solution, thereby reducing or eliminating problems caused by caustic effects near the injection site(s). The temporally controlled rate of flow and/or concentration of the treatment solution can be adapted in real time by feedback as the patient's measured pH returns closer to normal during treatment.

In an embodiment of the current invention, a total volume rate of injection of a treatment solution dispensed by a computer-controlled mixer/dispenser to an injection site of a patient is between about 0.1 microliter per minute to about 10 milliliter per minute.

In an embodiment of the current invention, a total delivered volume of a treatment solution dispensed by a computer-controlled mixer/dispenser to an injection site of a patient is between about 1 milliliter to about 1 liter.

In an embodiment of the current invention, a total delivered base resulting from delivery of a treatment solution dispensed by a computer-controlled mixer/dispenser to an injection site of a patient is between about 10 mEq to about 1000 mEq.

In an embodiment of the current invention, a volume rate of intake of an input solution used by computer-controlled mixer/dispenser to form a treatment solution is between about 0.1 microliter per minute to about 10 milliliter per minute.

In an embodiment of the current invention, a measurement of pCO2 or [CO₂] of a patient is used in a feedback loop by a computer-controlled mixer/dispenser to modify at least one of a base composition, a base concentration, a volume rate of dispensing, and a total delivered volume of a treatment solution to an injection site of a patient.

In an embodiment of the current invention, a computer-controlled mixer/dispenser and associated tubing, needles, and input solutions and containers are sterile.

In and embodiment of the current invention, at least one of an equation describing equilibrium concentrations and pressures described below (or a result thereof) is used by a computer program that directs a computer-controlled mixer/dispenser to select at least one of a base composition, a base concentration, a volume rate of dispensing, and a total delivered volume of a treatment solution that is mixed and dispensed to an injection site of a patient.

In an embodiment of the current invention, measured conditions of a patient connected to a computer-controlled mixing and dispensing system that are used by the mixing and dispensing system's software to adjust and control at least one of a composition, a concentration, a rate, and a location of injection of at least one of a base-treatment solution, a mixture of two or more base-treatment solutions, and a mixture of a base-treatment solution and a dispersion of pH-altering nanoparticles include: a heart rate, a blood pressure, a respiration rate, an electrocardio signal, a heart ejection factor, a brain-wave signal, a body temperature, an arterial blood pH, a venous blood pH, an arterial [HCO₃₋], a venous [HCO₃⁻], an arterial PCO2, a venous PCO2, an arterial [Na⁺], a venous [Na⁺], an arterial [Cl⁻], a venous [Cl⁻], an arterial [K⁺], a venous [K⁺], an arterial PO2, and a venous PO2.

In an embodiment of the current invention, if a patient is connected to an automated respiration system, then one or more signals system containing information about the composition of inhaled gas, composition of exhaled gas, frequency of respiration, and an effective volume per breath inhaled/exhaled are transmitted from said automated respiration to said computer-controlled mixing and dispensing system. Alternatively, said information about the settings of the automated respiration system are manually entered into said computer-controlled mixing and dispensing system if said automated respiration system is not equipped to transmit signals in a compatible manner. In an embodiment of the current invention, if said automated respiration system is equipped to receive control signals, then said computer-controlled mixing and dispensing system transmits one or more signals back to the automated respiration system, thereby changing said respiration parameters used by the automated respiration system to cause a change in respiration of the patient, in real-time in response to monitored changes in at least one of pH, PCO2, [HCO₃⁻], composition of base solution, and rate of administering base solution, that are measured, affected, and/or controlled by the computer-controlled mixing and dispensing system. Respiration of a patient can influence PCO2, thereby affecting pH and [HCO3-], so a treatment of an acidemic patient connected to an automated respiration system optimally involves both administration of base-solution by the computer-controlled mixing and dispensing system in coordination with appropriate alteration of the respiration parameters of the automated respiration system, as controlled by the computer-controlled mixing and dispensing system.

Modes of transmission of signals to and from a computer-controlled mixing and dispensing system can include at least one of: transmission by electrically conducting wire, transmission by fiber optical line, transmission by light waves, transmission by electromagnetic waves, and transmission by sound waves. For instance, in an embodiment of the current invention, electromagnetic waves in the form of wifi signals are used to transmit signals to and from a computer-controlled mixing and dispensing system and sensors of said computer-controlled mixing and dispensing system, pumps of said computer-controlled mixing and dispensing system, and a wireless electronic communication network (e.g. of a hospital). In an embodiment of the current invention, use of wireless communication, such as wifi signals or other electromagnetic and light signals, by the computer-controlled mixing and dispensing system facilitate the placement of said sensors and pumps relative to the patient without cumbersome wires or lines that are necessary for hard-wired electrical connections and fiber-optical connectors.

In an embodiment of the current invention, said computer-controlled mixing and dispensing system is used to treat a form of acidosis. Forms of acidosis that are treatable by said computer-controlled mixing and dispensing system include but are not limited to: metabolic acidosis, respiratory acidosis, lactic acidosis, ketoacidosis, dilutional acidosis, starvation acidosis, and diabetic acidosis.

A computer-controlled mixing and dispensing system according to some embodiments can be used to treat disorders in a patient other than acidosis, including but not limited to: renal failure, diarrhea, intoxication, rhabdomyolysis, diabetes, and poisoning. Such treatments can involve administration of at least one of a solution, a mixture of two or more solutions, and a mixture of a solution and a dispersion of particles.

A computer-controlled mixing and dispensing system according to some embodiments can be used to treat a patient suffering from alkylosis using a near-isotonic acid-treatment solution that is administered via said computer-controlled mixing and dispensing system.

A computer-controlled mixing and dispensing system according to some embodiments, said near-isotonic base-treatment solutions, and said dispersions of pH-influencing particles can be used to treat animal patients suffering from disorders including but not limited to acidosis in a veterinary medical context.

Herein, we refer to an “acidemic patient”, and we also refer to an “acidotic patient”. Our meaning of “acidemic patient” is a patient who has a condition of acidosis. Likewise, our meaning of “acidotic patient” is a patient who has a condition of acidosis. Thus, our use of the words “acidotic”, “acidemic”, and “acidemia” refer to a state of acidosis that may be in blood as well as in tissue of a patient. Herein, we refer to the pressure of carbon dioxide gas using notations including pCO2, pCO₂, PCO2, and PCO₂.

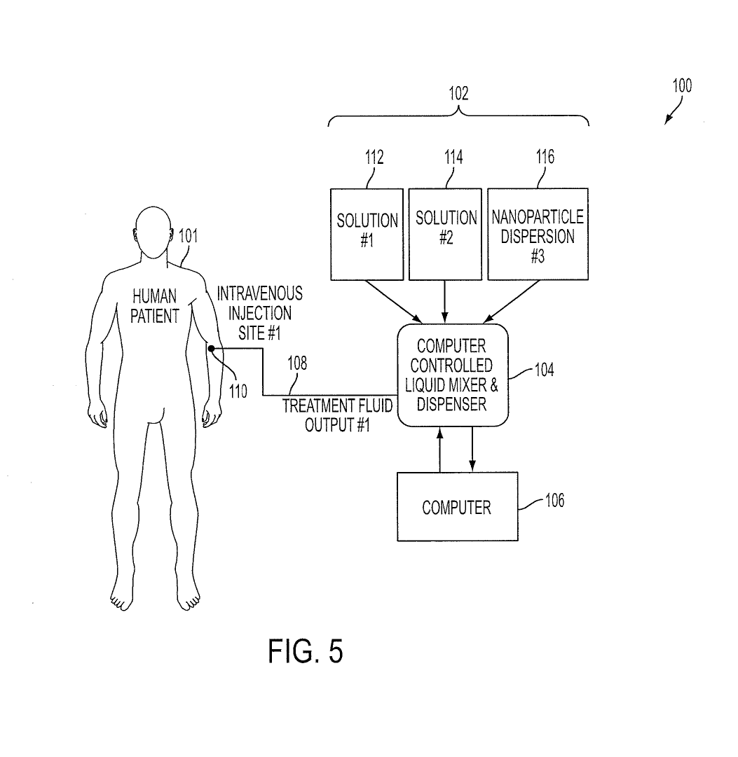

FIG. 5 is a schematic illustration of a system 100 for treating an acidotic patient 101 according to an embodiment of the current invention. The system 100 includes an intravenous-fluid supply system 102, an automated fluid mixer and dispenser 104 connected to the intravenous-fluid supply system 102 to receive at least one supply fluid therefrom, an electronic control system 106 configured to communicate with the automated fluid mixer and dispenser 104, and an intravenous line 108 fluidly connected to the automated fluid mixer and dispenser 104. The term “intravenous line” is intended to be broad to include the term “tube”, catheter, or any other structure that is suitable for delivering fluid to the acidotic patient's circulatory system. The intravenous line 108 includes an intravenous connecter 110 configured for injecting intravenous fluid dispensed from the automated fluid mixer and dispenser 104 intravenously into the acidotic patient 101. The electronic control system 106 is configured to control at least one of a total volume or a flow rate of the intravenous fluid to be injected into the acidotic patient's blood based on at least a measured pH of the acidotic patient's blood and based on a composition of the at least one supply fluid.

The embodiment of the intravenous-fluid supply system 102 illustrated in FIG. 1 is configured to be able to supply three solutions, or precursor solutions, 112, 114, 116. The intravenous-fluid supply system 102 is not limited to this example. In some embodiments, a single solution supply may be sufficient, in some embodiments, two solutions may be provided, or in other embodiments, more than three solutions and/or dispersions could be provided, as desired for a particular application.

The automated fluid mixer and dispenser 104 is illustrated as a combined assembly in FIG. 1. However, the automated fluid mixer and dispenser 104 can be separate mixer and dispenser components in some embodiments. The automated fluid mixer and dispenser 104 can include syringe pumps, such as, but not limited to syringe pumps by HAMILTON according to some embodiments of the current invention.

The electronic control system 106 can be, or can include, a computer according to some embodiments of the current invention. These can include, but are not limited to one of more work station, personal computer, tablet computer, smart phone, or other handheld, laptop, which could be local and/or networked over a local area network and/or the internet, for example. The electronic control system 106 can include data storage and/or memory and can be programmed to perform the control functions. The electronic control system 106 can be programmed through software and/or hardware such as, but not limited to ASICs and/or FPGAs. The electronic control system 106 can also be a separate component from the automated fluid mixer and dispenser 104, or could be integrated into a common package with at least some components of the automated fluid mixer and dispenser 104.

In some embodiments, the electronic control system 106 can be further configured to control at least one of the total volume or the flow rate of the intravenous fluid to be injected into the acidotic patient's blood based on at least one of a predicted pH effect, a predicted pCO2 effect, and a predicted bicarbonate anion concentration effect of the intravenous fluid to be injected.

In some embodiments, the electronic control system 106 can be further configured to control at least one of the total volume or the flow rate of the intravenous fluid to be injected into the acidotic patient's blood further based on a predicted effect of the intravenous fluid to be injected on an isotonic condition of the acidotic patient's blood.

In some embodiments, the at least one supply fluid from the intravenous-fluid supply system includes at least a first supply fluid and a second supply fluid, e.g., from 112 and 114. The electronic control system 106 can be further configured to provide control signals to the automated fluid mixer and dispenser 104 to mix the first and second supply fluids in a proportion based on at least the measured pH of the acidotic patient's blood. In some embodiments, pCO₂ and/or other measured data can be used by the electronic control system 106 to provide control signals to the automated fluid mixer and dispenser 104 to mix the first and second supply fluids. Although this refers to an embodiment in which two supply fluids are mixed, the general concepts of the current invention are not limited to this example. In some embodiments, a single solution can be provided, while in others more than three solutions can be mixed. In addition, one or more dispersions could be mixed with one or more solutions, and/or used separately as a single dispersion to be administered according to some embodiments of the current invention.

In some embodiments, the electronic control system 106 can be further configured to provide control signals to the automated fluid mixer and dispenser 104 to mix the first and second supply fluids in a proportion and a concentration based on a predicted effect of the intravenous fluid to be injected on an isotonic condition of the acidotic patient's blood. The first supply fluid can have a higher pH in a base range than a pH of the second solution according to some embodiments of the current invention. The second supply fluid can be an aqueous solution that includes dissolved sodium bicarbonate, and the first solution can be an aqueous solution that includes at least one of dissolved disodium carbonate, sodium hydroxide, or tris(hydroxymethyl)aminomethane, for example.

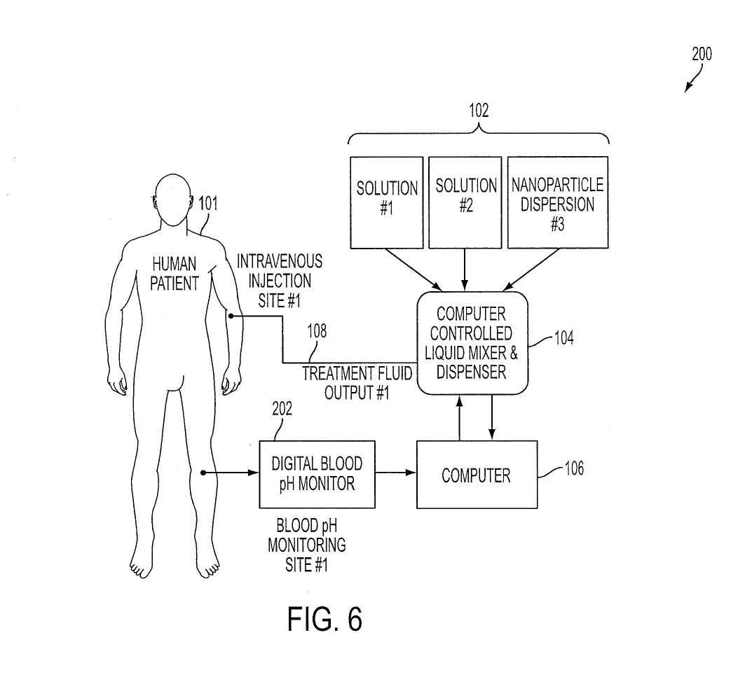

FIG. 6 is a schematic illustration of a system 200 for treating an acidotic patient 101 according to an embodiment of the current invention. The system 200 can share the same or similar components as the system 100 as indicated by the same reference numerals. The system 200 also includes a blood sensor system 202 configured to communicate with the electronic control system 106. The blood sensor system 202 can be or can include a digital blood pH monitor, for example. However, the blood sensor system 202 is not limited to only digital blood pH monitor. It can alternatively, or additional include pCO₂ sensors, for example. It can also include alternative or additional blood monitors. Furthermore, other sensor systems could also be included, such as, but not limited to blood pressure, electrocardiogram, etc. In some applications, without limitation, a blood gas and chemistry monitor by VIA MEDICAL is suitable.

In some embodiments, the blood sensor system 202 is configured to measure at least one property of the patient's blood in real time and provide sensor signals to the electronic control system. The signals can be provided by a wire and/or wirelessly in some embodiments. In this embodiment, the electronic control system 106 can provide control signals to the automated fluid mixer and dispenser such that at least one of mixing or dispensing by the automated fluid mixer and dispenser is based at least partially on real-time information from the blood sensor system.

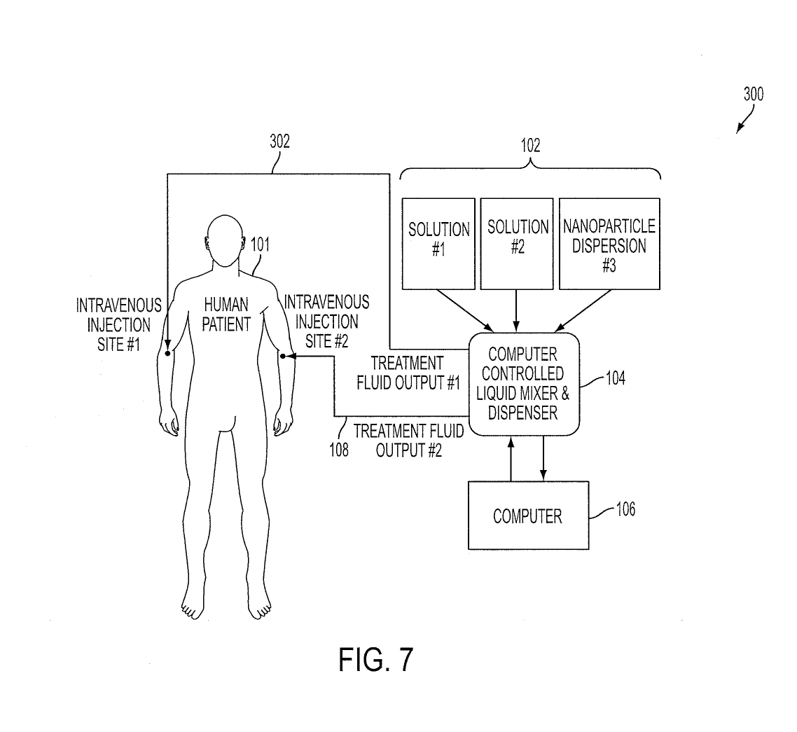

FIG. 7 is a schematic illustration of a system 300 for treating an acidotic patient 101 according to another embodiment of the current invention. The system 300 can share the same or similar components as the systems 100 and 200 as indicated by the same reference numerals. The system 300 also includes a second intravenous line 302 fluidly connected to the automated fluid mixer and dispenser 104. The second intravenous line includes a second intravenous connecter 304 configured for injecting second intravenous fluid dispensed from the automated fluid mixer and dispenser 104 intravenously into a second position in the acidotic patient. Although the embodiment of FIG. 7 has two intravenous lines; three, four or more could be included in other embodiments.

In some embodiments, intravenous-fluid supply system 102 may be loaded with a plurality of precursor solutions that when mixed by the automated fluid mixer and dispenser, provides an intravenous solution to be dispensed. In some embodiments, intravenous solution can include sodium bicarbonate and at least one of disodium carbonate, sodium hydroxide, and tris(hydroxymethyl)aminomethane dissolved in an aqueous solution such that the intravenous solution has a pH of at least 10 and a total concentration of osmolites within a near isotonic range. The aqueous solution can include disodium carbonate and sodium bicarbonate in a molar ratio of at least 2:1 in some embodiments. The intravenous solution can be essentially only disodium carbonate and sodium bicarbonate dissolved in the aqueous solution in a molar ratio of at least 2:1 in some embodiments.

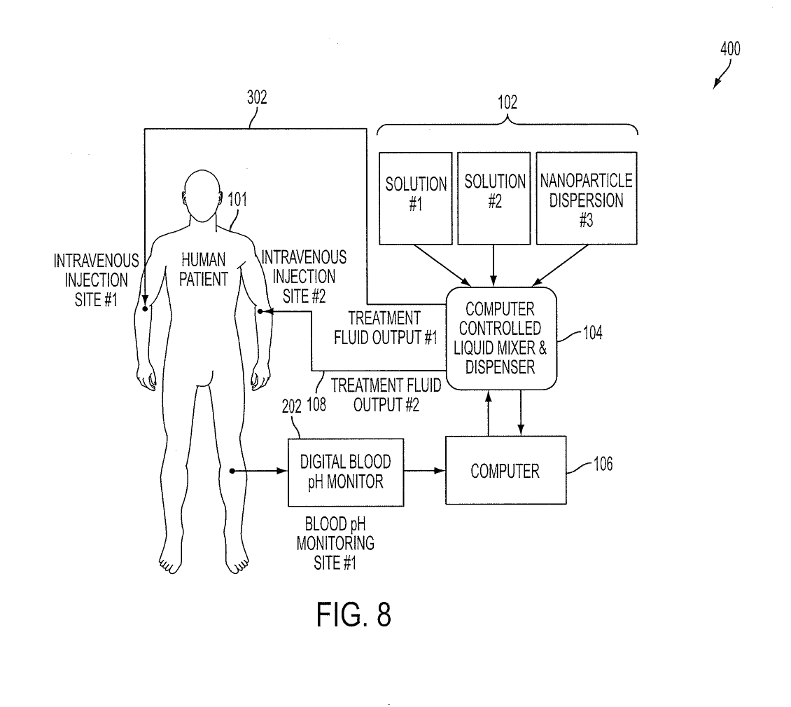

FIG. 8 is a schematic illustration of a system 400 for treating an acidotic patient 101 according to another embodiment of the current invention. The system 40 can share the same or similar components as the systems 100, 200 and 300 as indicated by the same reference numerals. The system 400 also includes a second intravenous line 302 fluidly connected to the automated fluid mixer and dispenser 104, such as in FIG. 7, as well as a blood sensor system 202 configured to communicate with the electronic control system 106, similar to the embodiment of FIG. 6.

In some embodiments, the computer-controlled mixing/dispensing system can also record values of actual liquid compositions and volumes dispensed to the different sites as a function of time, as well as recording measured signals from sensors that are connected to the mixing/dispensing system (e.g. in the computer's memory storage system). The recorded time history of measurements and injected liquid parameters, as well as real-time signals, can be used by the mixing/dispensing system to adjust and control the composition, rate, etc. of fluids delivered to the patient. Such recorded history can be a factor to adjust type, concentration, rate, etc. of fluids dispensed in some embodiments.

Another embodiment of the current invention is directed to an intravenous solution for treating acidosis. The intravenous solution includes sodium bicarbonate and at least one of disodium carbonate, sodium hydroxide, and tris(hydroxymethyl)aminomethane dissolved in an aqueous solution. The intravenous solution has a pH of at least 10 and a total concentration of osmolites within a near isotonic range. In some embodiments, the aqueous solution includes disodium carbonate and sodium bicarbonate in a molar ratio of at least 2:1. In some embodiments, the aqueous solution includes essentially only disodium carbonate and sodium bicarbonate in a molar ratio of at least 2:1.

Another embodiment of the current invention is directed to a method of treating acidosis. The method includes providing an intravenous solution for treating acidosis, and administering the intravenous solution intravenously to an acidotic patient. The intravenous solution has a pH of at least 10 and a total concentration of osmolites within a near isotonic range.

In some embodiments of the method of treating acidosis, the intravenous solution includes sodium bicarbonate and at least one of disodium carbonate, sodium hydroxide, and tris(hydroxymethyl)aminomethane dissolved in an aqueous solution. In some embodiments of the method of treating acidosis, the aqueous solution includes disodium carbonate and sodium bicarbonate in a molar ratio of at least 2:1. In some embodiments of the method of treating acidosis, the aqueous solution includes essentially only disodium carbonate and sodium bicarbonate in a molar ratio of at least 2:1.

In some embodiments, the method of treating acidosis further includes selecting the intravenous solution to have a composition based on at least one of a measured pH value, a measured pCO2 value, and a measured bicarbonate anion concentration of the acidotic patient.

In some embodiments, the method of treating acidosis further includes measuring a pH of the acidotic patient, and at least one of selecting or mixing the intravenous solution to have a composition based on the measuring the pH value of the acidotic patient. In some embodiments, the method can further include repeating the measuring and at least one of selecting or mixing a plurality of times to provide a real-time adjusted method of treating acidosis.

Another embodiment of the current invention is directed to an intravenous dispersion for treating acidosis. The intravenous dispersion includes a liquid and a plurality of particles dispersed in the liquid. Each particle of the plurality of particles has a maximum outer dimension of less than about 2 micrometers such that the particles can pass unhindered through capillary blood vessels of an acidotic patient being treated. Each of the plurality of particles includes at least one of a shell and a matrix material that dissolves at a predetermined rate within the acidotic patient's blood stream, and each of the plurality of particles includes a pH-influencing material that mixes in the acidotic patient's blood stream at a controlled rate while the at least one of the shell and the matrix material dissolves.

A rate of dissolution of the pH-influencing material can be controlled by at least one of a composition of the at least one of the shell and the matrix material, a structure of the at least one of the shell and the matrix material, a relative volume fraction of the pH-influencing material and the at least one of the shell and said matrix material, and an average size of the plurality of particles.

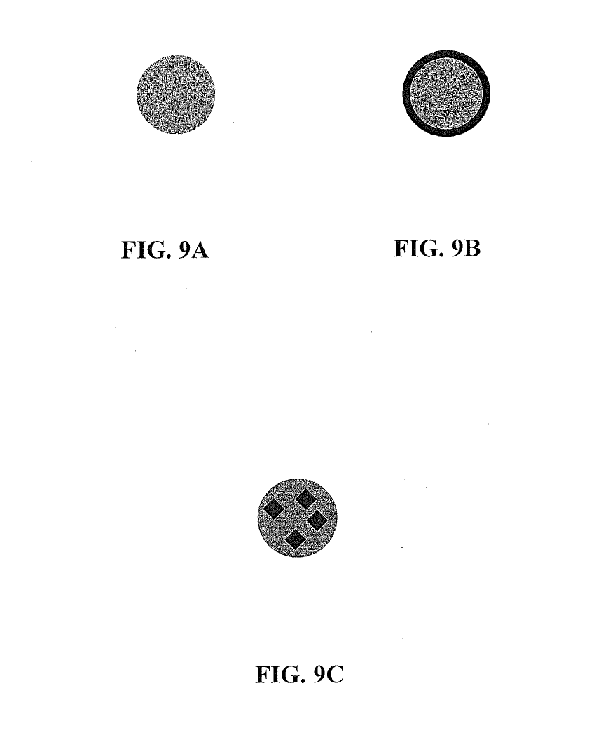

FIGS. 9A-9C show some embodiments of particles that can be used for producing dispersions according to some embodiment of the current invention. FIG. 9A shows an embodiment of a particle (core) composition. It can be one or more of the following:
a material that neutralizes hydronium ions or protons in a water-based liquid, such as blood
a material that dissolves in and/or dissociates in a water-based liquid to form a basic solution and raise pH
a base-functional material (e.g. a strong base, a weak base, a salt of a conjugate acid that functions as a base, an oxoacid containing an electropositive element that functions as a base)
a buffer material (e.g. to regulate pH to a desired value)
examples: sodium carbonate Na₂CO₃,
   sodium bicarbonate NaHCO₃
      sodium salts of carbonates are good candidates for core material because they naturally occur and are readily regulated and eliminated by the body
   sodium phosphate Na₃PO₄
   sodium hydroxide NaOH (a strong base)

The particle (core) structure can be spherical or non-spherical (e.g. a crystalline or polycrystalline grain), can be solid or porous, can be only partially composed of a base-functional material. The particle (core) size can be microscale or smaller so that it can be easily dissolved and will not get trapped in small capillaries. Nanoscale sizes can be desirable. Monodisperse or polydisperse size distributions can be suitable for particular applications.

FIG. 9B shows an embodiment of a coated particle that can be used for producing dispersions according to some embodiment of the current invention. The particle shell composition can be one or more of the following:
a protein, a sugar, a poly-peptide, an amphiphilic co-polymer
an albumin, a poly-nucleic acid, a polymer
a poly-(ethelene oxide), a poly-(ethylene glycol)
an enzymatically degradable biocompatible polymer
a material that creates a surface charge to stabilize particles against aggregation in an aqueous solution
a material that is pH sensitive and dissolves in water when the pH in the solution around it falls below a certain predetermined value
a material that is temperature sensitive and only dissolves in water when the temperature approaches body temperature
a material that inhibits the dissolution of the core material for a desired predetermined time after injection into the body of an organism, particularly a human being

The particle shell structure can be solid or porous, can be amorphous, crystalline, liquid crystalline, and/or polycrystalline. It may be desirable for the particle shell material to provide a repulsion between two coated particles and between a coated particle and a body structure (e.g. vein). The particle shell thickness can be microscale or nanoscale so that it can be dissolved in blood readily and will not get trapped in small capillaries, for example. Nanoscale sizes can be desirable; as well as monodisperse or polydisperse size distributions.

FIG. 9C shows an embodiment of a composite particle that can be used for producing dispersions according to some embodiment of the current invention. Examples of a composite nanoparticle composition include:
The pH-influencing material is at least partially obstructed by the matrix material from dissolving rapidly in an aqueous environment.
The pH-influencing material is shown here as small black diamonds inside a spherical grey matrix material.
For example, the pH-influencing material can be sodium bicarbonate or a hydrate thereof, and the matrix material can be a dextran. Both sodium bicarbonate and dextran are soluble in water, but the rate of dissolution of the dextran can be controlled through the molecular weight of the dextran, thereby controlling the rate of dissolution of the sodium bicarbonate.

In an embodiment of the current invention, the particle concepts of FIGS. 9B and 9C can be combined, resulting in a particle having a shell around a composite particle that exhibits a time delay prior to the dissolving of an internal pH-sensitive material, affected by a property of the shell, and a time-rate of dissolution of a pH sensitive-material, affected by a property of the matrix material.

According to an embodiment of the current invention, a treatment liquid containing dispersed nanoparticles (whether coated or not) and/or nanodroplets (whether encapsulated or not) is injected into the circulatory system of a patient suffering from acidosis. The primary component of this treatment liquid is typically water. These dispersed nanoparticles and/or nanodroplets contain pH-influencing materials, such as sodium carbonate or sodium bicarbonate, that can be carried by the patient's blood flow away from the injection site before completely dissolving or releasing all of said pH-influencing material, thereby alleviating caustic damage to the patient's tissue near the injection site. The aforementioned nanoparticles and/or nanodroplets can also be dispersed in ionic and/or molecular treatment solutions for acidosis, such as sodium bicarbonate and/or sodium carbonate solutions.

In treating acidosis, it could be desirable to directly reduce hydronium ion concentration by a nanoparticle containing a pH-influencing material that while the nanoparticle is dissolving in the blood as it is being transported in the patient's circulatory system, the released pH-influencing material can effectively neutralize some excess hydronium ions in the blood. For instance, a solid nanoscopic particle of sodium carbonate placed directly in the blood will dissolve and dissociate, yielding two sodium cations and one carbonate anion. The carbonate anion will rapidly take up a proton from a hydronium ion in the blood to form a bicarbonate anion, which is amphoteric and functions thereafter as a natural buffer in the blood. Provided the nanoscopic particle dissolves gradually yet fully as it is carried along in the blood, its prolonged dissolution as it travels through the patient's circulatory system will enable it to distribute the caustic exposure much more evenly throughout the patient than would a basic solution or a basic buffer that mixes more rapidly with the blood causing greater exposure of tissue to basic environment or abnormal pH near the injection site.

Nanoparticles of Na₂CO₃ (or e.g. hydrates thereof) that dissolve directly in the blood can enhance a therapeutic effect by neutralizing hydronium ions present in the blood and thereby raising blood pH and subsequently overall pH levels within cells throughout the body as well as providing a source of bicarbonate ions that are beneficial in further regulating body pH to a normal range.

In an embodiment of the current invention, examples of nanoparticles containing a material that influences pH (e.g. containing a base or base-producing material when dissolved in water) for the purpose of treating acidosis, are shown in FIGS. 9A-9C.

The sizes of the nanoparticles or nanodroplets can be fabricated and selected to provide a range of time for dissolution in the flowing bloodstream. Some polydispersity in the size distribution is beneficial, yet particles cannot be so large as to obstruct or get lodged in the circulatory system of the patient. It may be typically desirable to have nanoparticle or nanodroplet sizes (i.e. effective maximal distance spanning the particle or droplet) about or less than about 200 nm, since larger particles might create problems with circulation or could create issues with locally high regions of caustic pH if they might get stuck and dissolve in one fixed location.

Coatings of nanoparticles containing a pH-influencing material, such as a base-producing material Na₂CO₃, (or coatings that encapsulate nanodroplets which contain basic solutions) can be engineered in many ways that could be beneficial. An example is shown in FIG. 9B. Many advantageous types of coating materials could be used (e.g. a naturally bio-compatible material such as at least one of a polymer, a protein, a nutrient, an enzyme, a ribonucleic acid, a kinase, an albumin, a lipid, a lipo-protein, a glyco-protein, a sugar, a dextran, an amino acid, a glycine, a poly-peptide, a co-polypeptide, and an inorganic salt). The function of the coating can be to protect the injection site from highly caustic solutions that could otherwise form through too rapid a rate of dissolution of the particle material and thereby cause damage body tissue in the injection region. The coating also can inhibit attractions between nanoparticles and/or nanodroplets that can cause destabilization of the dispersion the nanoparticles or nanodroplets prior to administration in the patient and inhibits attractive interactions that would promote sticking of the particles to venous and arterial walls, as well as other bodily structures, which could result in strong local exposure that might lead to highly localized tissue damage where the particles have become stuck. A range of thicknesses of the coating material and a range of solubilities of the coating material could be used so that particles dissolve at different locations in the body, thereby better distributing the caustic load more evenly.

In an embodiment of the current invention, a coating material is designed to dissolve at a specific pH (i.e. if the blood pH begins to drop below a certain value such as 7.2), thereby providing a feedback mechanism to further regulate pH.

In an embodiment of the current invention, a coating material is designed to transform by at least one of melting, dissolving in water, and reacting when the temperature is raised from about 25 degrees C. to about 37 degrees C. Such a mechanism would cause a coating material that is stably protecting the core material against dissolution at room temperature; the resulting temperature-dependent transformation effectively then deprotect the core material when the nanoparticle enters the body of the patient and the nanoparticle's temperature rises to body temperature, allowing the core material (which contains a pH-influencing material) to then dissolve further downstream from the injection site in the patient.

In an embodiment of the current invention, a coating material that can dissolve, be metabolized, and/or be enzymatically broken down by human blood, is selected to confer a property of stabilization of nanoparticles against aggregation.

In an embodiment of the current invention, high-throughput production of nanoparticles of sodium carbonate is accomplished by creating a fine aerosol of microscale droplets of an aqueous solution of sodium carbonate (e.g. at 0.1% by mass) and evaporating the water from the aerosol by heating the aerosol using a hot-air convective blower. Production of a fine aerosol of microscale to nanoscale droplets of solution can be accomplished using a variety of devices, such as atomizers, nebulizers, ultrasonic agitators, and thermal-spray nozzles. As an option, the nanoparticles in the airstream of the convective blower can then be deposited into a solvent for subsequent coating by a coating material that is soluble in that solvent. Another aerosol of the nanoparticles in that solvent+coating material can be formed, and the solvent can be evaporated, yielding coated core-shell nanoparticles. Alternatively, a surface coating can be grown on nanoparticles in the airstream of the convective blower prior to deposition into an aqueous solution.

In another embodiment of the current invention, a composite nanoparticle containing two or more materials in a configuration other than a core-shell configuration, can also be efficacious in controlling the rate of dissolution and therefore spatial and temporal distribution of a pH-influencing material. For instance, a nanoparticle containing a smaller amount of solid sodium carbonate that is evenly distributed within a larger amount of sodium bicarbonate can be formed by evaporating a nebulized aerosol of an aqueous solution of sodium carbonate mixed with sodium bicarbonate. An example of a nanoparticle composition and structure, in which small domains of a pH-influencing material (e.g. base or base-functional material) are in a matrix material to make up a composite nanoparticle, for treating acidosis is shown in FIG. 9C.

In an embodiment of the current invention, a matrix material that can dissolve, be metabolized, and/or be enzymatically broken down by human blood, and said matrix material in addition confers a property of stabilization of nanoparticles against aggregation.

In an embodiment of the current invention, a matrix material remains undissolved in water at a pH equal to or greater than about 7.3 yet dissolves readily when the pH drops below about 7.3.

In an embodiment of the current invention, a matrix material is designed to transform by at least one of melting, dissolving in water, and reacting when the temperature is raised from about 25 degrees C. to about 37 degrees C. Such a mechanism would cause a matrix material, which stably protects at least some pH-influencing material against dissolution at room temperature; to transform, thereby effectively deprotecting the interspersed pH-influencing material which can then dissolve in an aqueous environment when said nanoparticle enters the body of the patient and said nanoparticle's temperature rises to body temperature. Thus, this temperature-sensitive composition of the matrix of a nanoparticle can gradually deprotect the pH-influencing material after the temperature in the environment of the nanoparticle changes (e.g. upon administration to a patient), so that it dissolves further downstream from the injection site in the patient.

Alternatively, in yet another embodiment, a nanoparticle containing a pH-influencing material, such as solid sodium bicarbonate, dispersed in a matrix of a pH-neutral material, such as a biocompatible polymer (e.g. a dextran), can be formed by taking an aqueous solution of a dextran and sodium bicarbonate, forming an aerosol of microscale to nanoscale droplets, and evaporating the water to form a plurality of discrete nanoparticles that have a composite composition. In this composition, the rate of time release of the carbonate is governed by the relative amounts of dextran to sodium bicarbonate; more dextran slows down the dissolution of the sodium bicarbonate, thereby regulating the rate of release of the pH-influencing material.

In another embodiment of the current invention, a nanoparticle containing a pH-influencing material, a non-pH-influencing biocompatible matrix material, and a non-pH-influencing bio-compatible amphiphile material are added to water to form an aqueous solution in order to produce a plurality of nanoparticles by evaporation of an aerosol of droplets of said aqueous solution. During the evaporation of the water, the amphiphile preferentially spatially segregates towards the surface of the nanoparticle. The solid nanoparticles are then dispersed in a liquid (e.g. water), and the amphiphile functions to stabilize the nanoparticles against aggregation.

In another embodiment of the current invention, a plurality of solid nanoparticles containing a pH-influencing material are dispersed into an aqueous solution, and that aqueous solution is administered into a patient's circulatory system immediately after dispersion and before said plurality of nanoparticles can completely dissolve and release all of a pH-influencing material contained within them.

In an embodiment of the current invention, the composition of a nanoparticle for treating acidosis contains between about one percent to one hundred percent of at least one of sodium carbonate, sodium bicarbonate, a hydrate of sodium carbonate, a hydrate of sodium bicarbonate, and sodium hydroxide.

In an embodiment of the current invention, the composition of a nanoparticle for treating acidosis contains between about one percent to about ninety percent of at least one of sodium carbonate, sodium bicarbonate, a hydrate of sodium carbonate, a hydrate of sodium bicarbonate, and sodium hydroxide and also contains about ninety-nine percent to about ten percent of a pH-neutral biocompatible matrix material that dissolves in human blood.

In an embodiment of the current invention, the composition of a nanoparticle for treating acidosis contains between about ten percent to about ninety-five percent of at least one of sodium carbonate, sodium bicarbonate, a hydrate of sodium carbonate, a hydrate of sodium bicarbonate, and sodium hydroxide and also contains about ninety percent to about five percent of a biocompatible coating material that dissolves in human blood.

In an embodiment of the current invention, the composition of a nanoparticle dispersion for treating acidosis is water and a plurality of nanoparticles containing a pH-influencing material, wherein a volume fraction of nanoparticles containing a pH-influencing material lies in the range from about 10⁻⁶ to about 0.3.

In an embodiment of the current invention, the composition of a nanoparticle dispersion for treating acidosis is water and a plurality of nanoparticles containing a pH-influencing material, wherein a volume fraction of nanoparticles containing a pH-influencing material lies between about 0.01 and 0.0001 and a pH-influencing material within said nanoparticles is at least one of sodium carbonate, sodium bicarbonate, a hydrate of sodium carbonate, a hydrate of sodium bicarbonate, and sodium hydroxide.

In an embodiment of the current invention a composite nanoparticle containing a pH-influencing material and a matrix material, is coated with a coating material to form a hybrid coated-composite nanoparticle for treating acidosis.

The general concepts and improvements underlying these new materials and methods, while focusing on acidosis in particular as an example, can be extended to treat regulatory ailments affecting the chemistry of the entire body, other than acidosis, such as alkylosis. These approaches can potentially be combined to further optimize treatments for a variety of disorders and ailments.

### REFERENCES

Cooper D J, Walley K R, Wiggs B R and Russell J A. Bicarbonate does not improve hemodynamics in critically ill patients who have lactic acidosis. Ann Intern Med 112: 492-498, 1990.
Kraut, J. A. and Madias, N. E. Treatment of acute metabolic acidosis. Nephrol Nat Rev. 2012.
Kraut J A and Kurtz I. Use of base in the treatment of acute severe organic acidosis by nephrologists and critical care physicians: results of an online survey. Clin Exp Nephrol 10: 111-117, 2006.
Kraut J A and Madias N E. Metabolic acidosis: pathophysiology, diagnosis and management. Nat Rev Nephrol 6: 274-285, 2010.
Leung J M, Landow L, Franks M, Soja-Strzepa D, Heard S O, Arieff A I and Mangano D T. Safety and efficacy of intravenous Carbicarb in patients undergoing surgery: comparison with sodium bicarbonate in the treatment of metabolic acidosis. Crit Care Med 22: 1540-1549, 1994.
Shapiro J I, Elkins N, Logan J, Ferstenberg L B and Repine J E. Effects of Sodium-Bicarbonate, Disodium Carbonate, and A Sodium-Bicarbonate Carbonate Mixture on the P-Co2 of Blood in A Closed-System. Journal of Laboratory and Clinical Medicine 126: 65-69, 1995.
J. A. Kraut and I. Kurtz, Use of Base in the Treatment of Severe Acidemic States. Am J Kidney Diseases. 2001; 38: 703-727.
J. A. Kraut, Effect of Metabolic Acidosis on Progression of Chronic Kidney Disease. Am J Physiol Renal Physiol. 2011; 300: F828-F829.
G. F. Filley and N. B. Kindig, Carbicarb, an Alkalinizing Ion-Generating Agent of Possible Clinical Usefulness. Trans Am Clin Climatol Assoc. 1985; 96: 141-153.
R. M. Bersin and A. I. Arieff, Improved hemodynamic function during hypoxia with Carbicarb, a new agent for the management of acidosis. Circulation. 1988; 77:227-233.
D. W. Oxtoby, H. P. Gillis, and A. Campion, Principles of Modern Chemistry, 6th ed., Thomson Brooks/Cole (2008).

### Measured pH of a Solution of Sodium Carbonate and Sodium Bicarbonate

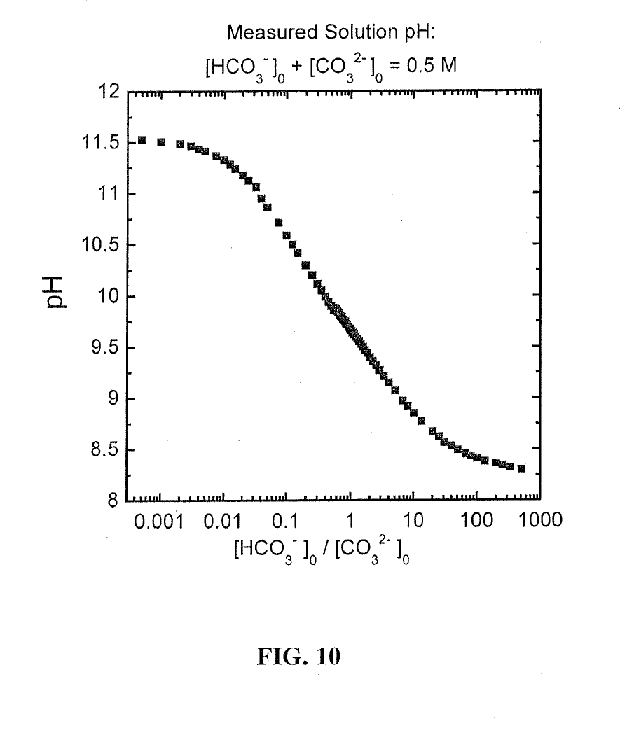

The measured pH of a solution of sodium carbonate and sodium bicarbonate at a fixed 0.50 M=[HCO₃⁻]₀+[CO₃²⁻]₀ has been measured using a digital pH meter calibrated with appropriate standard solutions. The results of these measurements, performed by titration, are shown below in FIG. 10.

Overall, the function given by the measured pH([HCO₃⁻]₀/[CO₃²⁻]₀) has the same shape as the predicted values in FIG. 1. Also, within experimental uncertainty, the measured value of pH for the sodium bicarbonate solution in the limit of large [HCO₃⁻]₀/[CO₃²⁻]₀ in FIG. 10 is in good agreement with the predicted value in FIG. 1.

However, at small [HCO₃⁻]₀/[CO₃²]₀, the value of the measured pH is about 11.5, which is smaller than the predicted pH of about 12.0. A likely reason why the measured pH is somewhat lower than the predicted pH is that the sodium carbonate used in the experiment has limited purity (i.e. is common utility grade rather than ultra-pure reagent grade) and likely contains a small but non-negligible percentage of sodium bicarbonate.

According to an embodiment of the current invention, a measured relationship between equilibrium pH and solution composition, such as the measured pH([HCO₃⁻]₀/[CO₃²⁻]₀) as displayed in FIG. 10, is used to select a composition of a solution for treating an acidemic patient suffering from acute metabolic acidosis.

### Nanoparticles Containing a Base Material for Treating Acidosis

Pure sodium bicarbonate solutions at high concentrations, typical of what is currently administered in treating acute metabolic acidosis, also have very high concentrations of CO₂. For instance, a pure NaHCO₃ solution at 0.5 M has a pCO2 approaching about 100 mm Hg=100 torr and a pH of about 8.3. This high pCO2 is undesirable, because the typical pCO2 in blood of a normal individual is only about 40 torr. The extra pCO2 means that more dissolved CO₂ in the blood will be converted to carbonic acid, which will give up a proton and convert to the bicarbonate anion, thereby tending to lower the pH and negating at least part of the excess hydroxide present in the basic sodium bicarbonate solution that raises pH. So, at least one of the reasons why sodium bicarbonate solutions are not very effective or desirable in treating acidosis is because these solutions introduce significant quantities of dissolved CO₂, and this large amount of dissolved CO₂ will actually counteract some of the pH-raising tendency of the bicarbonate anion.

It would be much more beneficial, instead, to raise pH in the patient's blood, and ultimately in the patient's cells, by introducing nanoparticles containing strong base, such as Na₂CO₃, into the flowing blood and having these nanoparticles release Na⁺ and CO₃²⁻ ions into the blood gradually in a controlled manner. Each one of the released CO₃²⁻ ions would effectively eliminate one hydronium ion in the blood, thereby directly lowering the proton concentration and raising pH. In addition, the product of this proton-accepting reaction is the bicarbonate ion HCO₃⁻, which is the main component of the currently administered bicarbonate solution and is known also to slightly raise pH, be safe in humans, and offers excellent permeability into cells. The rate of dissolution and dissociation of a strong base, such as Na₂CO₃, in blood can be controlled by creating nanoparticles containing the strong base. These nanoparticles satisfy several requirements: they are small enough to freely travel in a significant portion of the patient's circulatory system and they are designed to release base material to raise pH in a controlled manner that avoids continuous exposure of a localized area of a patient's tissue to a very high pH over a sustained period of time that would lead to undesirable tissue damage in that localized area. For instance, by injecting nanoparticles containing Na₂CO₃ that are circulated in blood throughout the acidemic patient, rather than a concentrated solution of Na₂CO₃, it is possible to avoid tissue damage near the site of injection that would otherwise result from intravenous injection of a strongly basic solution. Moreover, nanoparticles that continuously release small enough concentrations of the base material Na₂CO₃ in a controlled manner while flowing in the circulatory system can reduce or prevent tissue damage as well as damage to red blood cells, proteins, or other important components in the blood. As the nanoparticles release base material while circulating in the blood of the patient, before they are completely gone, they can more evenly distribute the proton accepting material throughout the body, thereby reducing tissue damage due to prolonged high caustic exposure of basic solutions near the injection site. Whereas a pure solution of sodium carbonate is likely to cause tissue damage if injected in the standard manner, if a way can be found to introduce CO₃²⁻ ions in the patient in a distributed manner in time and space, then the tissue damage due to a more caustic agent such as Na₂CO₃ could be reduced or eliminated. Moreover, the composition of the non-base material is biologically compatible and can be readily used and/or eliminated by the patient.

In an embodiment of the current invention, a nanoparticle that contains one or more smaller nanogranules of a strong base material, such as sodium carbonate, in a non-base material, such as at least one of a matrix material and a coating material. In some cases, it is desirable for the non-base material to be chosen so as to maintain the integrity of the nanoparticles and inhibit release of the strong base when the aqueous dispersion of nanoparticles is stored prior to administration, and only after intravenous administration, the non-base material is then chosen so that it is readily dissolved or degraded after entering the patient. As examples, the non-base material of the nanoparticles can be enzymatically degraded by naturally occurring enzymes in the blood, the non-base material can be dissolved in certain acedmic pH range in the patient, and/or the non-base material can be dissolved when heated above a certain temperature, such as body temperature. In some cases, a base material by itself in the form of a nanoparticle dispersion of pure base material would dissolve so readily in an aqueous environment that it would become a solution very rapidly, and could not be stored as a dispersion. The use of a non-base material in the composition of the nanoparticle therefore permits long-term stability of the nanoparticle dispersion outside of the patient, and the type, quantity, and structure of the non-base material in a nanoparticle are chosen through a fabrication process to confer a desired rate of release of the base material in a flowing environment of a biofluid, such as blood, in a living patient.

### Example Calculation of a Dispersion of Nanoparticles Containing a Strong Base

Consider an aqueous dispersion of monodisperse nanoparticles; each nanoparticle has a radius a and contains a base-material (e.g. sodium carbonate) and a non-base material (e.g. a dextran). The base material has a mass density ρ_{b} and a molecular weight of M_{Wb}. The base material inside the nanoparticle is protected against rapid dissolution in water by the non-base material, which is typically structured in a manner to be at least one of a matrix material and a coating material. The structure and type of non-base material controls the rate of dissolution of the base material when the nanoparticle is administered in an aqueous dispersion to a patient suffering from acidosis. In the case of intravenous administration, controlling the rate of dissolution of the base material and thus the release of acid-neutralizing material facilitates a more even distribution of the base material throughout the patient's bloodstream, thereby reducing undesirable complications and side-effects due to spatially localized regions of higher-than-normal pH over a sustained period of time. Such high local pH over a sustained period of time, which can cause significant tissue damage, typically occur near the administration site when caustic solutions are administered intravenously to patients suffering from acute metabolic acidosis.

For simplicity, we consider a nanoparticle to be a sphere (although the current invention is not limited only to spherical shapes), so the volume of a nanoparticle is given by Vₚ=4πa³/3. Within a nanoparticle, the volume of base material is V_{bp} and the volume of non-base material is V_{nbp}, so the volume fraction of base material in the particle is given by ψ=V_{bp}/(V_{bp}+V_{nbp})=V_{bp}/Vₚ. Thus, the volume of base in a single nanoparticle is V_{bp}=ψVₚ=4πψa³/3. The mass of base in a single nanoparticle is thus m_{bp}=ρ₀V_{bp}. The number of moles of base material in a single nanoparticle is thus n_{bp}=m_{bp}/M_{Wb}=(4π/3)ψa³(ρ₀/M_{Wb}), and the number of molecules of base material in a single nanoparticle is simply N_{bp}=n_{bp}N₀, where N₀ is Avogadro's number.

As an example, a nanoparticle suitable for treating acidosis contains the base material sodium carbonate Na₂CO₃ as solid nanogranules, smaller in size than the nanoparticle itself; these nanogranules are contained within in a non-base matrix material that controls the rate of dissolution of the nanogranules and release of base material into a surrounding aqueous medium, such as blood. For Na₂CO₃, ρ₀=2.54 g/cm³ and M_{Wb}=105.98 g/mol, a nanoparticle having a=50 nm and ψ=0.3 has about 3.8×10⁻¹⁸ mol of Na₂CO₃, equivalent to about 2.3×10⁶ molecules of Na₂CO₃ per nanoparticle, yielding the same number of CO₃²⁻ anions per nanoparticle when the Na₂CO₃ within the nanoparticle has completely dissolved and dissociated in the aqueous medium.

Suppose a patient has acute metabolic acidosis, and the patient's blood pH is 7.1, rather than the desired normal pH of 7.4. Recognizing that the total volume of body fluid is significantly more than the volume of blood in a patient, so that basing a calculation on only blood volume may yield an underestimate, we provide an estimate of the required number of nanoparticles, N_{particles,req}, that would be necessary to change the patient's blood pH (and not the rest of the body fluids) from 7.1 to 7.4 as follows. In calculating a first estimate, we initially consider only the excess hydronium ions in the blood, without regard to the patient's blood and/or body tissues, which can effectively act as a source of additional protons through chemical reactions. Thus, this first estimate represents an estimate of a minimum number of particles, not necessarily the total number of particles that would be needed to completely treat an acidemic patient. Also, in making this estimate, we account only for the primary proton-accepting reaction of CO₃²⁻ after it is released from the nanoparticles, and not the additional beneficial action of the HCO₃₋ anion that is the resulting product, which also acts to raise the pH of an acidemic patient.

The average volume of a patient's blood is assumed to be V_{blood}=5.0 L. The concentration of hydronium ions (or equivalently protons) in the patient's blood at pH=7.1 is about 7.9×10⁻⁸ M, whereas the desired concentration at pH=7.4 is about 4.0×10⁻⁸ M. So, the desired reduction in concentration of hydronium ions is Δ[H₃O⁺]=3.9×10⁻⁸ M, corresponding to a desired reduction of ΔN_{H3O+}=Δ[H₃O⁺]V_{blood} N₀=1.2×10¹⁷ hydronium ions in the blood. Because each CO₃²⁻ ion released by the nanoparticle will effectively accept a proton from a hydronium ion in the blood to form a bicarbonate anion HCO₃⁻, (or take a proton from a water molecule, leading to a hydroxide ion that will in turn neutralize a hydronium ion), we estimate that the total number of nanoparticles required to obtain the desired pH change in the blood will be: N_{particles,req}=1.2×10¹⁷/2.3×10⁶=5.2×10¹⁰ nanoparticles. This corresponds to a total volume of nanoparticles required to treat the blood of V_{particles,req}=N_{particles,req} Vₚ=2.7×10⁻⁵ cm³. This volume is quite small and simply provides a rough estimate of an approximate lower limit of what might be required to begin to raise an acidemic patient's blood pH towards a normal level, since the patient's blood and body tissues can act as a buffer, thereby requiring higher total dosage levels that the rough lower limit that is estimated here.

In designing an aqueous dispersion of nanoparticles to treat acidosis, in many cases, it would be desirable, but not necessary, for the dispersion of nanoparticles to be dilute (i.e. having a low volume fraction φ of nanoparticles), so the viscosity of the dispersion is not far above that of at least one of water and blood. A lower volume fraction of particles can facilitate rapid mixing of the administered dispersion with blood. As an example, a volume fraction of nanoparticles in water of about φ=0.05 is dilute enough to have a desirable viscosity that is not much greater than that of water itself, where φ=V_{particles,req}/(V_{particles,req}+V_{w}), where V_{w} is the volume of water in the dispersion. Thus, using numbers corresponding to the extreme lower limit, estimated above, the volume of the dispersion V_{disp}=V_{particles,req}+V_{w}=V_{particles,req}/φ=5.4×10⁻⁴ cm³, much less than the volume of blood in the patient. Even reducing the volume fraction of the nanoparticle dispersion down to φ=10⁻⁴, which could be advantageous in certain circumstances, since the viscosity of the dispersion is very close to that of water, would yield a total volume of the aqueous dispersion of only V_{disp}=0.27 cm³, still a very small volume to administer to a patient. This simplistic estimate does not account for the buffering capacity of the patient's blood or body tissues; a wide variety of chemical reactions in the acidemic patient will be generating more hydronium ions as the nanoparticles are releasing the base material over time.

Even dispersions having smaller volume fractions of nanoparticles containing strong base materials in solid form, such as sodium carbonate or sodium hydroxide, could be administered to an acutely acidemic patient to effectively raise the patient's pH the desired amount. Also, considering other body fluids, the volume of 5 L used in these calculations may be somewhat small compared to an approximate adult total body fluid volume of 40 L (i.e. 25 L is typically intracellular and 15 L is typically extracellular). If a total body fluid volume of an adult is used in the above calculations, then an estimate of a lower limit of the total volume of an aqueous dispersion of nanoparticles containing sodium carbonate at φ=10⁻⁴ would still be only about 2.2 cm³.

Since the above estimates neglect the buffering capacity of the blood and body tissues, we provide an estimate that is typically required to fully treat acidosis in an acidemic patient. It is typically necessary to consider the total number of moles of a base material that is needed in order to alter a patient's pH from an acidemic state to a normal state. For sodium bicarbonate solutions, administration of 500 mEq or more of the bicarbonate anion can be required to raise the pH of a patient suffering from acute metabolic acidosis to a normal level. As previously mentioned, solutions of sodium bicarbonate are not highly effective as a treatment, in part because of the high levels of dissolved CO₂ that can convert to carbonic acid. So, an effective treatment of an acidemic patient using nanoparticles containing a stronger base such as Na₂CO₃ typically requires only smaller total administered dose as compared to a weaker base such as NaHCO₃.

As an example of an embodiment of the current invention, we calculate the total required number of nanoparticles, the total required volume of dispersion of nanoparticles, and the volume fraction of nanoparticles in the dispersion that is required to treat a patient suffering from acute metabolic acidosis assuming that the total dose of strong base required to treat the patient is 100 millimoles. We consider nanoparticles containing Na₂CO₃, and the required number of moles of base is n_{b,req}=0.100 mol. The number of moles of base per particle, assuming ψ=0.51 (e.g. for spherical core-shell nanoparticles having an outer radius a=50 nm and an inner radius of aᵢ=40 nm within which the Na₂CO₃ is contained, where the shell of the nanoparticle is a non-base material), is simply n_{bp}=(4π/3)a³ ψρ_{b}/M_{wb}=6.42×10⁻¹⁸ mol. Thus, the number of nanoparticles required for the total dose is N_{particles,req}=n_{b,req}/n_{bp}≈1.56×10¹⁶. This corresponds to a total required particle volume of V_{particles,req}=N_{particles,req} Vₚ=8.15 cm³. To keep the volume fraction φ of nanoparticles in an aqueous dispersion at a dilute level, we choose a volume of water to be 191.85 mL, yielding a total volume of the dispersion of 200.0 mL. Thus, the volume fraction of nanoparticles is φ=8.15 mL/200 mL=0.041, which is less than 5% and is dilute. Thus, a reasonable volume of 200 mL of a dilute dispersion of 50 nm radius spherical nanoparticles containing Na₂CO₃ at ψ=0.51 and φ=4.1% can be used to administer a total dose of 100 mmol of a strong base to an acidemic patient.

An advantage of using a nanoparticle containing a strong base is that as the nanoparticle base material dissolves and dissociates in the patient's flowing bloodstream, it gradually releases ions (e.g. an anion such as CO₃²⁻ or OH⁻) as it flows along, and these ions act as bases to reduce the hydronium ion concentration directly (e.g. by forming HCO₃⁻ or H₂O, respectively) while avoiding the destruction of tissue near the site of injection into the patient as would be caused by a very caustic solution. Thus, whereas a total of 500 mEq of sodium bicarbonate solution may need to be administered to a patient suffering from acute metabolic acidosis in order to obtain the desired pH increase to treat extreme acidemic symptoms, yielding a large injected volume of a concentrated bicarbonate solution, by contrast, a smaller volume of a dispersion of nanoparticles containing a strong base, such as sodium carbonate, is typically necessary to achieve the same desired pH change by the same type of administration via intravenous injection.

In an embodiment of the current invention, the total time required for a nanoparticle containing a base-material to fully dissolve in human blood circulating in a live human patient at a temperature of about 37 C is less than about twenty-four hours. In an embodiment of the current invention, the total time required for a nanoparticle containing a base-material to fully dissolve in human blood circulating in a live human patient at a temperature of about 37 C is less than about two hours.

Alternatively, in an embodiment of the current invention, the rate of administration of the dispersion of nanoparticles is controlled and is the dominant factor in determining the rate of pH-raising action of base-material into the blood. In an embodiment of the current invention, the total time required for a nanoparticle containing a base-material to fully dissolve in human blood circulating in a live human patient at a temperature of about 37 C is less than about two minutes, and the volume rate of injection of the dispersion of nanoparticles is controlled to achieve the desired pH change in a desired period of time of administration.

In an embodiment of the current invention, a dispersion of nanoparticles containing a base material is an input to a computer-controlled mixer/dispenser system designed for administering a time-varying and/or spatially varying mixture of an acidosis treatment fluid.

Although we have mentioned water as being the continuous phase for the composition of a dispersion of nanoparticles containing a base material for use in the treatment of acidosis, a solution, such as a saline solution, could also be used as the continuous phase of the dispersion, provided that the ions present in the saline solution do not cause aggregation of the nanoparticles in the dispersion.

In an embodiment of the current invention, a nanoparticle for treating acidosis has a base material and a non-base material that are in a bicontinuous structure.

In an embodiment of the current invention, a nanoparticle for treating acidosis has a solid base material and a non-base material that is at least one of a solid, a liquid, and a liquid crystal, wherein said non-base material controls a rate of dissolution of said base material into an aqueous environment.

In an embodiment of the current invention, a nanoparticle suitable for treating acidosis is formed by: making a mixture of at least one of a solution and a dispersion of a non-base material with at least one of a solution and a dispersion of a base material in a liquid solvent, creating an aerosol of said mixture such that the diameters of the largest droplets in the aerosol are less than about ten microns, and evaporating said solvent.

Results of Calculations of pH and pCO₂ of an Aqueous Solution of Sodium Bicarbonate and Sodium Hydroxide

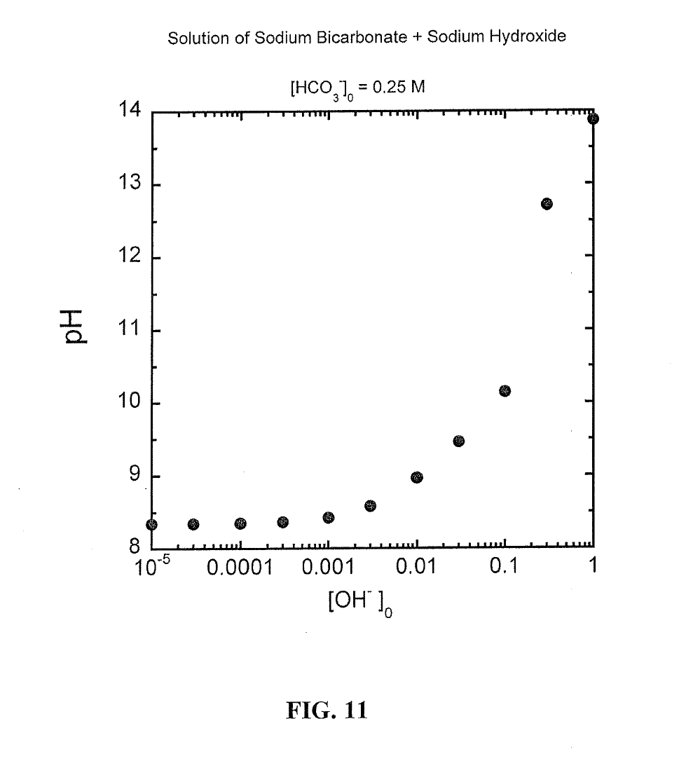

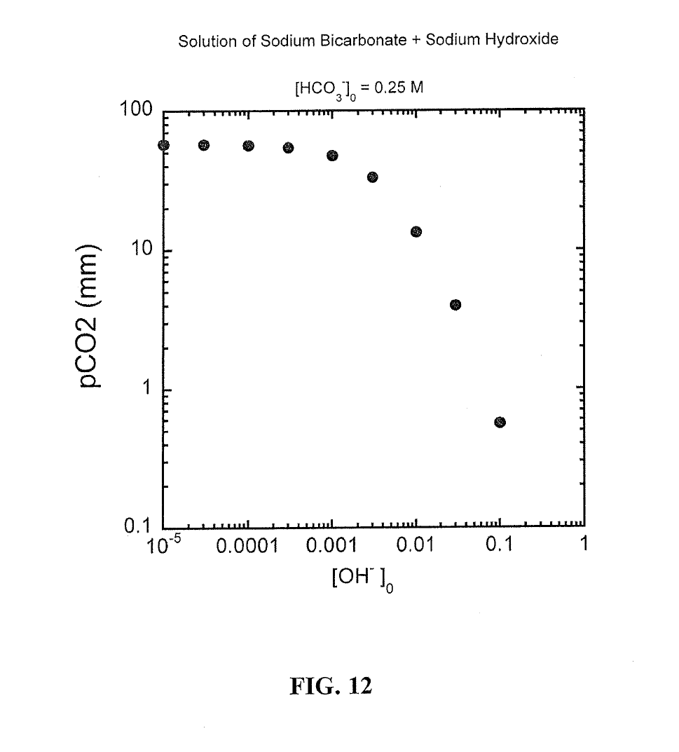

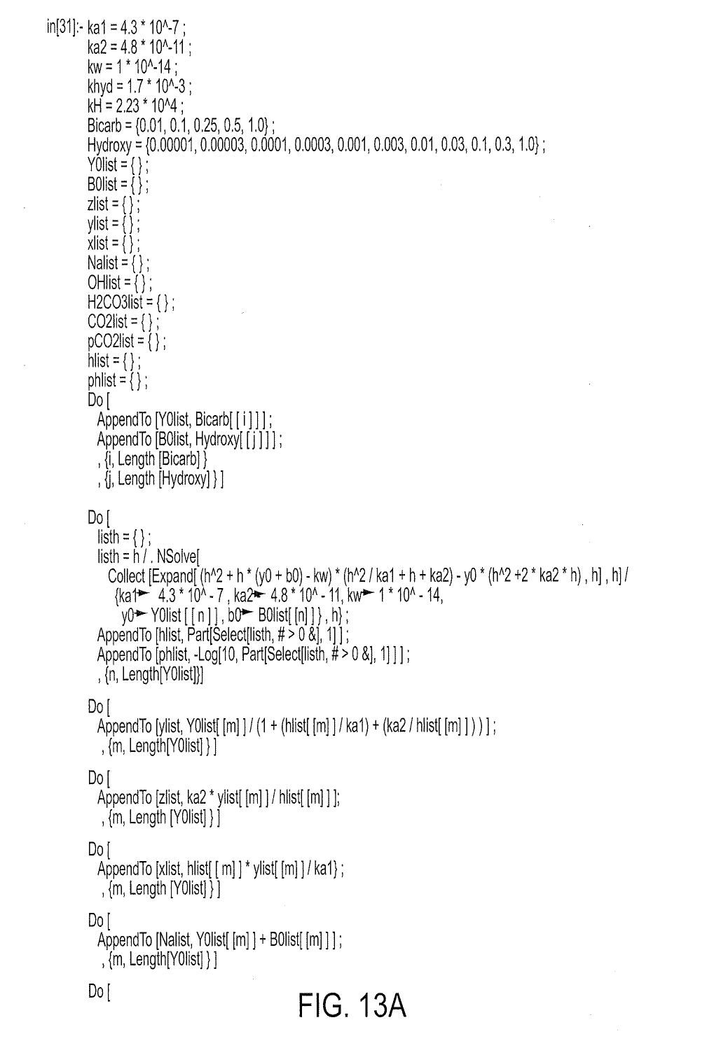

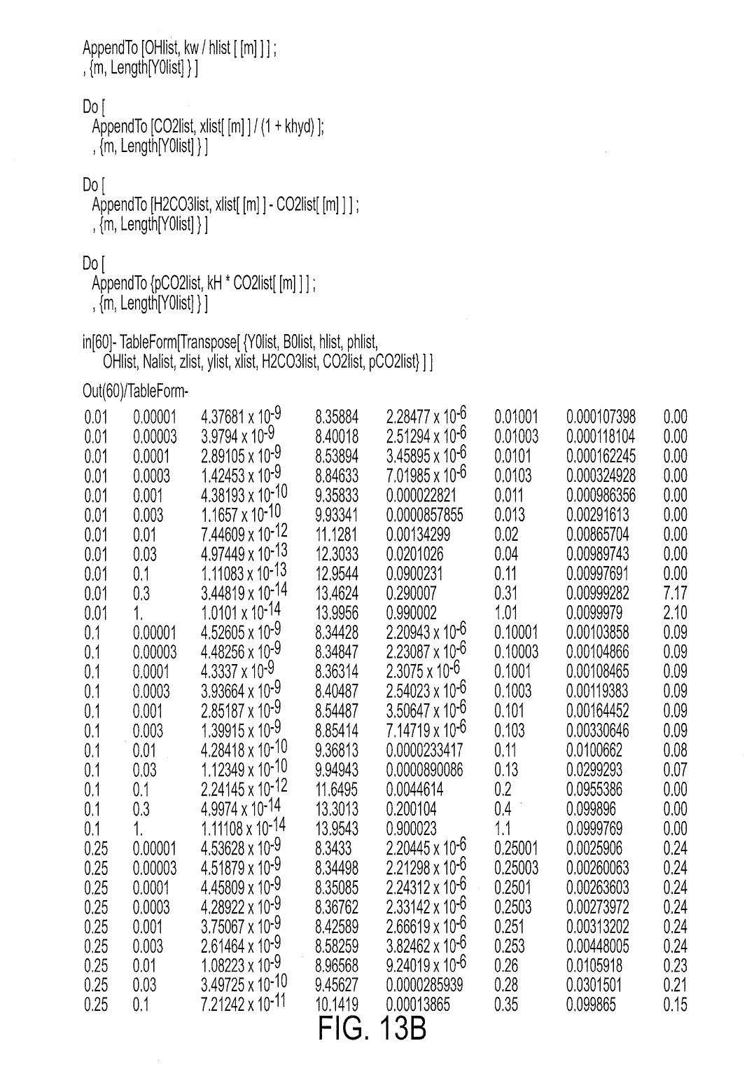

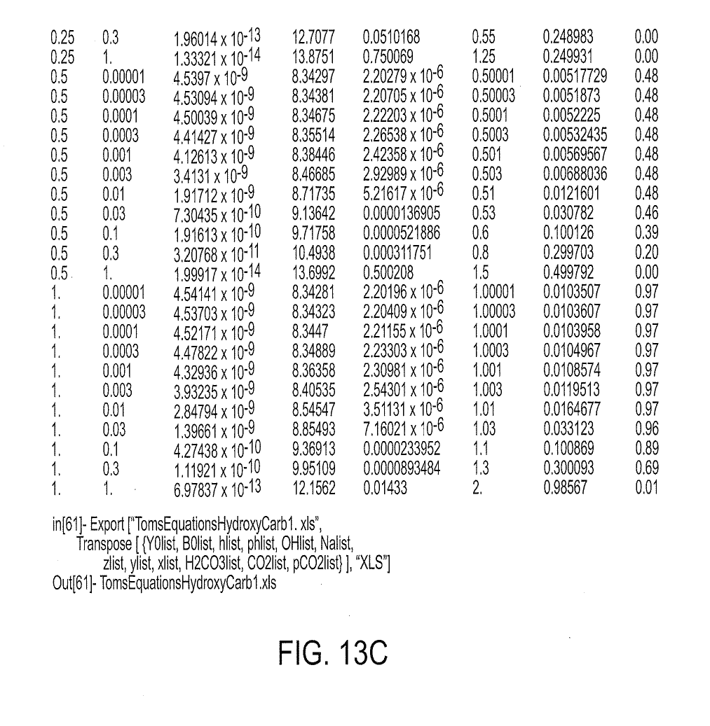

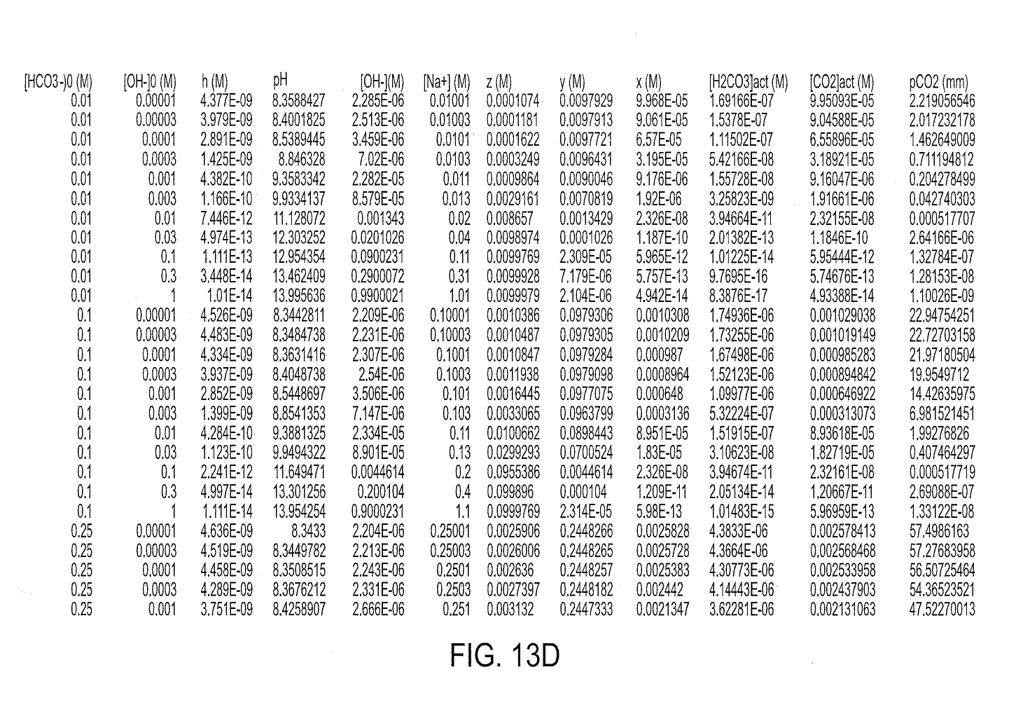

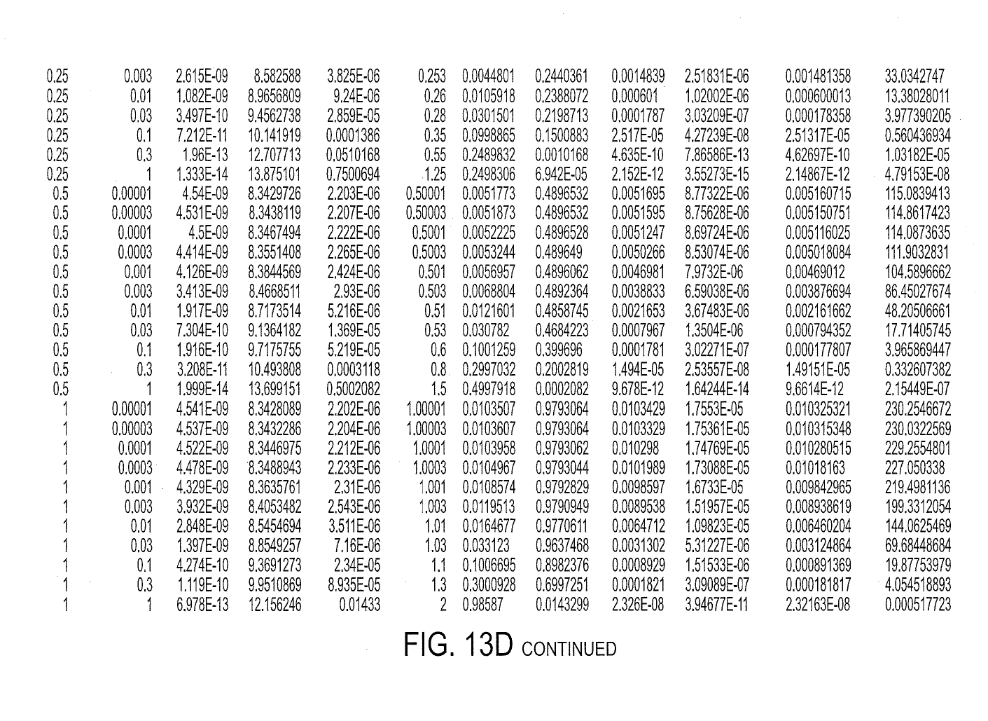

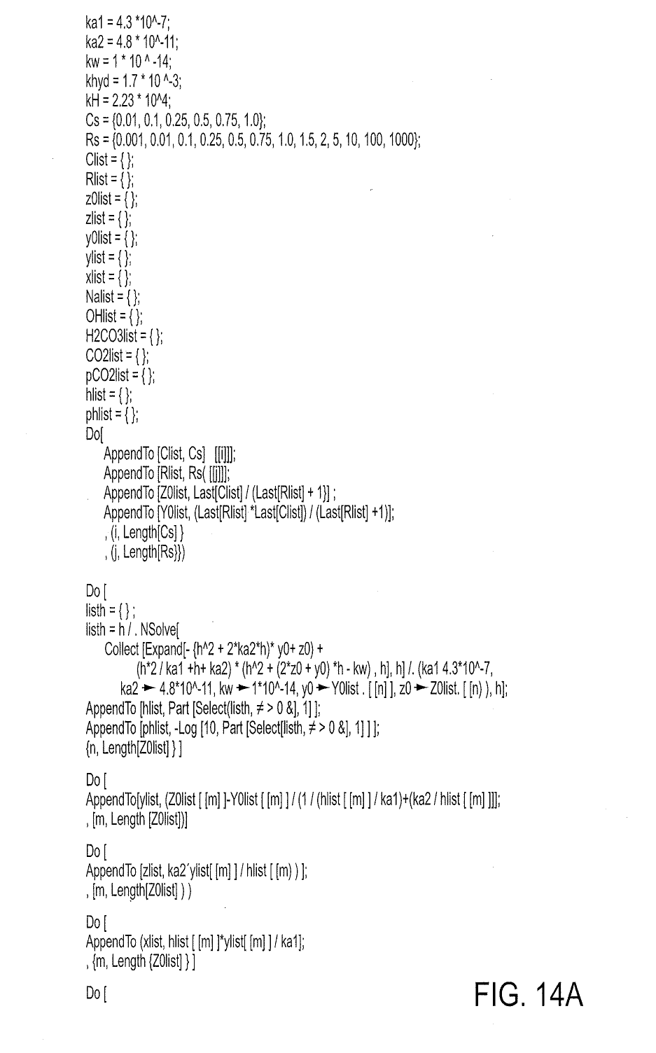

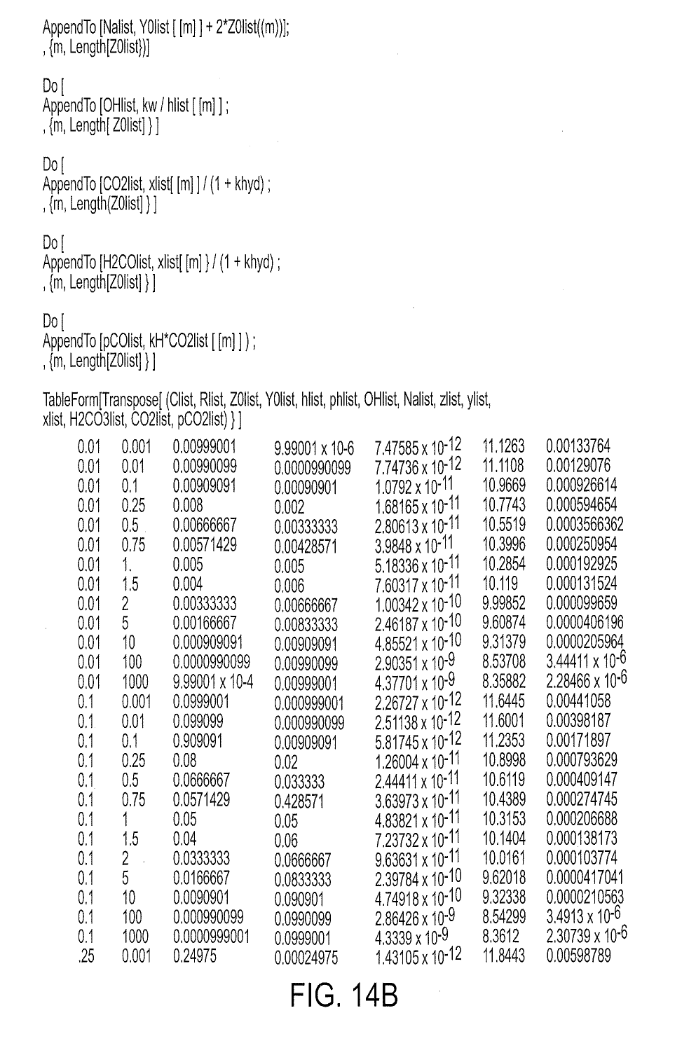

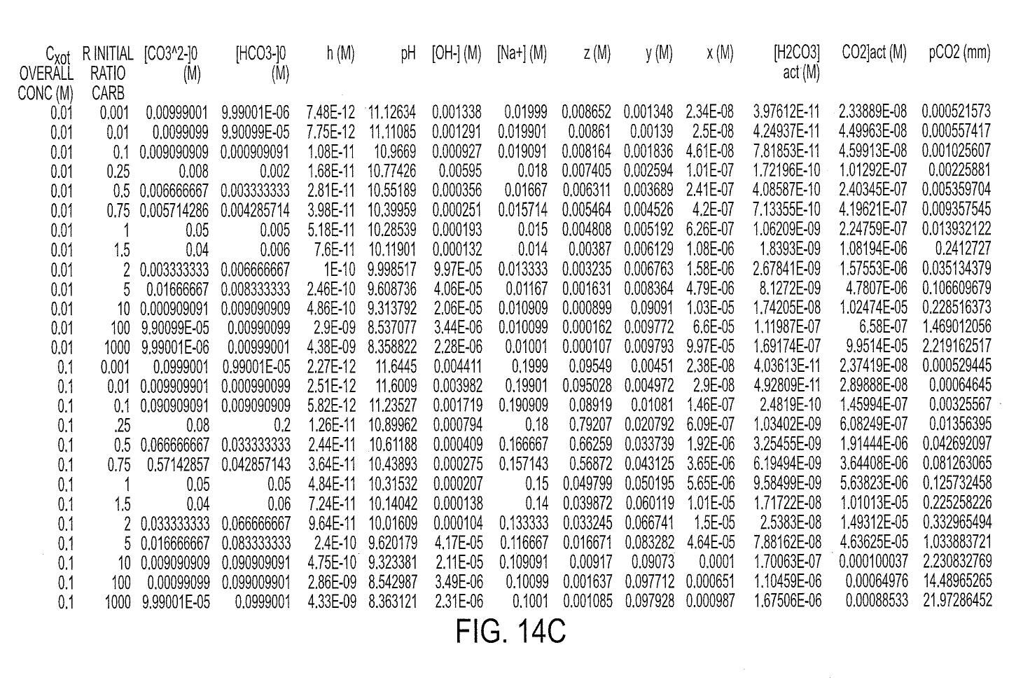

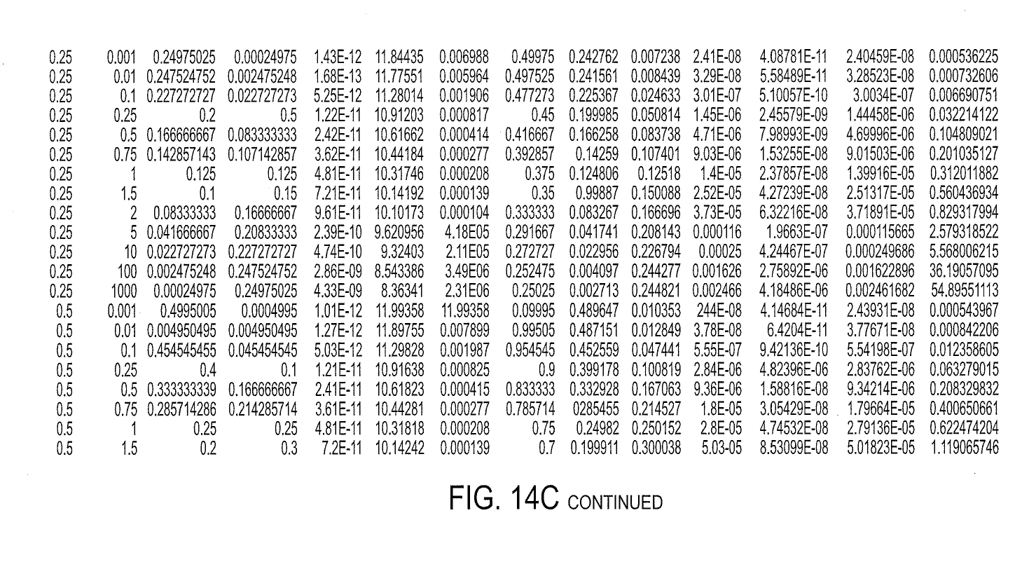

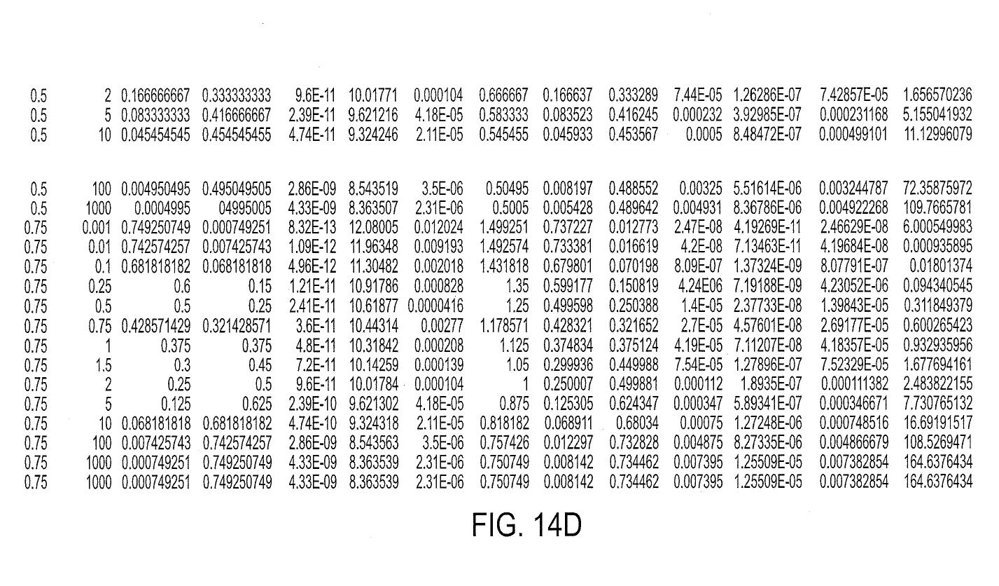

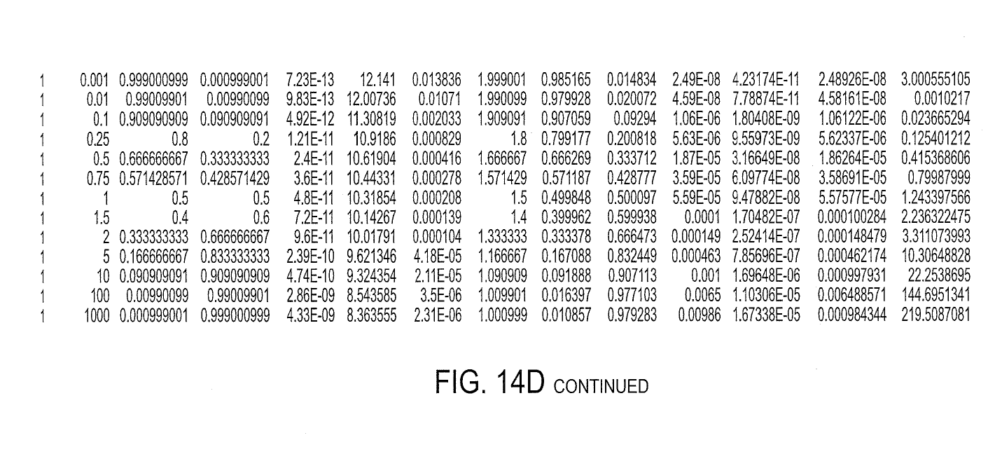

Below, we present the results of calculations of the equilibrium pH and pCO₂ of an aqueous solution of sodium bicarbonate and sodium hydroxide in FIGS. 11 and 12, respectively, when the initial concentration of sodium bicarbonate (and therefore the bicarbonate anion) is fixed at [HCO₃⁻]₀=0.25 M. The equations used to make these calculations are presented under the title of “Alternative Formulation”.

Thus, according to FIG. 11, a higher pH of the solution can be obtained with a modest addition of sodium hydroxide initially. According to an embodiment of the current invention, a solution of sodium bicarbonate and sodium hydroxide at particular [HCO₃⁻]₀ and [OH⁻]₀ is selected to treat an acidemic patient using a functional dependence of pH predicted by equilibrium equations such as those used to generate FIG. 11.

Thus, according to FIG. 12, a desirable reduction in pCO2 can be obtained by combining a sodium hydroxide and a sodium bicarbonate solution, especially as the amount of [OH⁻]₀ is increased. According to an embodiment of the current invention, a solution of sodium bicarbonate and sodium hydroxide at particular [HCO₃⁻]₀ and [OH⁻]₀ is selected to treat an acidemic patient using a functional dependence of pCO₂ predicted by equilibrium equations such as those used to generate FIG. 12.

Solutions of sodium bicarbonate and sodium hydroxide offer potentially valuable characteristics. The pH is adjustable to a higher value than that of a pure sodium bicarbonate solution through selection of at least one of [HCO₃⁻]₀ and [OH⁻]₀. Likewise, the pCO₂ can be adjusted to be lower than that of pure sodium bicarbonate solutions. In addition, for the same pH, there is a lower sodium load (i.e. concentration) introduced into the patient, as compared to solutions of sodium carbonate and sodium bicarbonate, since NaOH releases only one Na⁺ ion for every OH⁻ ion when it dissociates, whereas Na₂CO₃ releases two Na⁺ ions for every CO₃²⁻ anion.

In an embodiment of the current invention, the volume rates of input solutions and concentrations of input solutions of at least a solution of sodium bicarbonate and sodium hydroxide to a multi-input fluid mixer/dispenser are controlled by a computer in a time-varying manner so as to select and adjust at least one of pH, pCO₂, and [Na⁺] of a resulting output fluid delivered to an acidemic patient.

### Alternative Formulation:

### Solution of Sodium Bicarbonate and Sodium Hydroxide for Treating Acute Metabolic Acidosis

A certain amount of sodium bicarbonate is added to neutral water, yielding an initial concentration of the bicarbonate anion [HCO₃⁻]₀≈y₀. Also, a certain amount of sodium hydroxide is added to the same solution, yielding an initial concentration of the hydroxide anion [OH⁻]₀=b₀, where we assume that b₀=[OH⁻]₀>>10⁻⁷M (the concentration of hydroxide anions in neutral water is 10⁻⁷ M). The initial concentrations of the carbonate anion and carbonic acid are also zero:

[H₂CO₃]=x₀=0

[CO₃²⁻]=z₀=0.

The initial chemical equations, immediately representing/following dissolution and dissociation are:

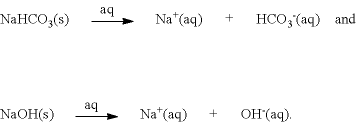

Thus, the initial concentration of the sodium cation is:

[Na⁺]=[HCO₃⁻]₀+[OH⁻]₀=y₀+b₀.

Since sodium is ineffective in acid-base chemistry, the equilibrium concentration of the sodium cation is simply the initial concentration:

[Na⁺]=[Na⁺]₀=y₀+b₀.

Now that the initial conditions have been well defined, we can consider the set of equations governing chemical equilibrium. These are:

[H₃O⁺]=h (concentration of hydronium ions)

[OH⁻]=K_{w}/h where K_{w}=10⁻¹⁴ (water auto-ionization)

[Na⁺]=y₀+b₀

Conservation of carbonate species requires:

[HCO₃⁻]₀=[H₂CO₃]+[HCO₃⁻]+[CO₃²⁻],

or y₀=x+y+z. (Recall: x=[H₂CO₃] & z=[CO₃²⁻]).

Charge neutrality requires:

[Na⁺]+[H₃O⁺]=[HCO₃⁻]+2[CO₃²⁻]+[OH⁻],

or (K_{w}/h)−h=b₀+y₀−y−2z.

From the carbonate equilibria, equations from the law of mass action are:

Ka₁=hy/x where Ka₁=4.3×10⁻⁷

Ka₂=hz/y where Ka₂=4.8×10⁻¹¹.

Substituting these two equations into the equation for the conservation of carbonate species yields:

y=y₀/[1+(h/Ka₁)+(Ka₂/h)].

Substituting the two equations above involving the law of mass action into the charge neutrality equation yields:

y=[y₀+b₀+h−(K_{w}/h)]/(1+2Ka₂/h).

Since the two expressions for y in terms of h must be equal, we obtain a quartic equation in the hydronium ion concentration:

[y₀+b₀+h−(K_{w}/h)][1+(h/Ka₁)+(Ka₂/h)]=y₀(1+2Ka₂/h).

So, 0=[h²+h(y₀+b₀)−K_{w}][(h²/Ka₁)+h+Ka₂]−y₀(h²+2Ka₂h).

This quartic equation is solved using Mathematica, similarly as has been done before, for various y₀ & b₀.

### pH of an Aqueous Solution of Sodium Bicarbonate and Sodium Carbonate

When sodium bicarbonate NaHCO₃ (or a hydrate thereof) is added to neutral water, it dissolves and dissociates:

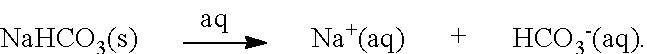

This reaction goes to completion, so the initial quantity of NaHCO₃ added to neutral water is [HCO₃⁻]₀ (i.e. the initial concentration of the bicarbonate anion HCO₃⁻).

When disodium carbonate Na₂CO₃ (or a hydrate thereof) is added to neutral water (pH=7.00), it dissolves and dissociates:

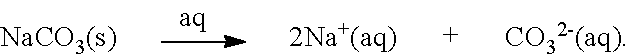

This reaction also goes to completion, so the initial concentration of carbonate anion CO₃²⁻, given by [CO₃²⁻]₀, is determined by a 1:1 molar ratio based on the disodium carbonate added.

Conservation of matter then requires that the initial concentration of sodium [Na⁺]₀ is:

[Na⁺]₀=[HCO₃⁻]₀+2[CO₃²⁻]₀.

Since Na⁺ is ineffective in acid-base chemistry, the equilibrium concentration of Na⁺ is:

[Na⁺]=[Na⁺]₀.

Given the initial concentrations of [HCO₃⁻]₀ and [CO₃²⁻]₀, which are dictated by the quantities of sodium bicarbonate and disodium carbonate, respectively, added to the neutral water, it is possible to solve for the equilibrium pH using a set of simultaneous equations for six variables:

[H₃O⁺]=h

[OH⁻]=K_{w}/[H₃O⁺]=K_{w}/h, where K_{w}=10⁻¹⁴ (from water auto-ionization)

[Na⁺]=[Na⁺]₀=[HCO₃⁻]₀+2[CO₃²⁻]₀

[H₂CO₃]=x

[HCO₃⁻]=y

[CO₃²⁻]=z

For simplicity, we define the following initial concentrations as:

[HCO₃⁻]₀=y₀

[CO₃²⁻]₀=z₀

[H₂CO₃]₀=0=x₀ (since no carbonic acid was added initially).

We can write equations of conservation of matter of the carbonate species, charge neutrality, and the deprotonating reactions of carbonic acid and the bicarbonate anion.

Conservation of carbonate species yields (at equilibrium):

[HCO₃⁻]₀+[CO₃²⁻]=[HCO₃⁻]+[H₂CO₃]+[CO₃²⁻],

simplifying to: x=(y₀−y)+(z₀−z). (Eq. 1)

Charge neutrality yields (at equilibrium):

[Na⁺]+[H₃O⁺]=[HCO₃⁻]+2[CO₃²⁻]+[OH⁻],

simplifying to: (K_{w}/h)−h=(y₀−y)+2(z₀−z). (Eq. 2)

Deprotonation of carbonic acid proceeds according to:

H₂CO₃(aq)+H₂O(l) [symbol] H₃O⁺(aq)+HCO₃⁻(aq)

with Ka₁=4.3×10⁻⁷=[H₃O⁺][HCO₃⁻]/[H₂CO₃]

according to the law of mass action.

This simplifies to Ka₁=hy/x. (Eq. 3)

Deprotonation of the bicarbonate anion proceeds according to:

HCO₃⁻(aq)+H₂O(l) [symbol] H₃O⁺(aq)+CO₃²⁻(aq)

with Ka₂=4.8×10⁻¹¹=[H₃O+][CO₃²⁻]/[HCO₃⁻].

This simplifies to Ka₂=hz/y. (Eq. 4)

Thus, there are four equations Eq. 1, Eq. 2, Eq. 3, & Eq. 4, and four unknowns x, y, and z.

We can rewrite Eq. 3 and Eq. 4 as:

x=hy/Ka₁ & z=Ka₂y/h

Substituting these relationships into Eq. 1 yields

hy/Ka₁=(y₀−y)+(z₀−Ka₂y/h),

which can be solved to obtain y in terms of h:

y=(y₀+z₀)/[1+(h/Ka₁)+(Ka₂/h)].

Substituting relationships for x and z into Eq. 2 yields

(K_{w}/h)−h=(y₀−y)+2(z₀−Ka₂y/h),

which can also be solved to obtain y in terms of h:

y=[y₀+2z₀+h−(K_{w}/h)]/(1+2Ka₂/h).

The right hand sides of the two equations for y must be equal, so

(1+2Ka₂/h)(y₀+z₀)=[1+(h/Ka₁)+(Ka₂/h)][y₀+2z₀+h−(K_{w}/h)].

This yields a 4^{th} order (quartic) polynomial in h, which is simplified to:

h⁴/Ka₁+[1+(y₀+2z₀)/Ka₁]h³+[Ka₂−(K_{w}/Ka₁)+z₀]h²−(K_{w}+Ka₂y₀)h−(Ka₂K_{w})=0.

Quartic equations can be solved, and the physical solution for h=[H₃O⁺] will be positive and real. Other imaginary or negative solutions can be discarded.

Once h is known, y, x, and z can also be calculated, yielding other equilibrium concentrations. These equations are provided above.

The value of x calculated in the previous equations actually accounts for the sum of the dissolved carbonic acid plus dissolved carbon dioxide.

x=[H₂CO₃]_{calc} (combined)

To separate out actual CO₂(aq) and H₂CO₃(aq), it is necessary to consider the so-called “hydration” equilibrium reaction of CO₂ in water:

CO₂(aq)+H₂O(l) [symbol] H₂CO₃(aq).

The law of mass action then implies:

K_{hyd}=[H₂CO₃]_{act}/[CO₂]_{act}, where K_{hyd}=1.7×10⁻³ at room temp.

Knowing x=[H₂CO₃]_{calc}=[H₂CO₃]_{act}+[CO₂]_{act},

then [CO₂]_{act}=[H₂CO₃]_{calc}/(1+K_{hyd})

and [H₂CO₃]_{act}=[H₂CO₃]_{calc}−[CO₂]_{act}.

The actual concentrations of CO₂ and H₂CO₃ can then be reported.

Once [CO₂]_{act} has been determined then it is straight forward to use Henry's Law to calculate the pressure of CO₂, pCO2:

pCO2=K_{H}[CO₂]_{act}

Where K_{H}=2.23×10⁴ mm/M is the Henry's Law value for CO₂ in H₂O at room temperature. Pressures are then expressed in mm Hg when [CO₂]_{act} is specified in molar units.

### EXAMPLES

The following examples help explain some concepts of the current invention. However, the general concepts of the current invention are not limited to the particular examples.

Acute metabolic acidosis is defined as a reduction in pH and serum bicarbonate ion concentration in blood below their respective normal ranges, typically lasting from about a minute to a few days. Acute metabolic acidosis is associated with cellular dysfunction and an increase in mortality. This cellular dysfunction is related to a decrease in the pH of the interstitial and cellular compartments.

In current clinical practice, acute metabolic acidosis is commonly treated by administering a highly concentrated aqueous solution of a weak base, such as sodium bicarbonate (NaHCO₃), which is injected into the circulatory system of a patient having an acidemic state. In the common treatment, the molarity of the solution of a weaker base is high (i.e. hypertonic), typically around or above one molar, because such administered solutions of weaker bases typically have a pH that is less than about 8.5 and are relatively ineffective at raising pH when administered at lower concentrations that are not hypertonic. In a typical solution of 1.0 M sodium bicarbonate, after dissociation, there are 2.0 M of osmolite species (i.e. 1.0 M of the sodium ion Na+ and 1.0 M of the bicarbonate ion HCO₃⁻). This 2.0 M osmolite concentration lies far beyond the isotonic concentration of osmolite species in human blood, around 300 mM. In clinical studies, a hypertonic solution of sodium bicarbonate has been shown to be non-optimal in treating acute metabolic acidosis, and such solutions have been demonstrated to depress cardiovascular function.

Clinicians and medical researchers have documented the ineffectiveness, and even adverse consequences, of administering the standard high-concentration sodium bicarbonate solutions to patients having acidemic states. In particular, the standard, high concentration, commonly available solutions of sodium bicarbonate have been shown to increase the pressure of carbon dioxide, PCO2, in blood. Raising PCO2 is typically undesirable, since hydrolysis of the additional carbon dioxide in the blood can subsequently lead to acid formation, thereby limiting the effectiveness of solutions of sodium bicarbonate in raising the pH of a patient's blood into the normal range that would alleviate the patient's state of acidemia.

Past research on high concentration solutions of equimolar mixtures of sodium bicarbonate and disodium carbonate (Na₂CO₃), called “carbicarb” (333 mM NaHCO₃, 333 mM Na₂CO₃), have revealed that blood pH could be raised while simultaneously lowering blood PCO2. However, the total osmolite concentration of the carbicarb solution mixture is 1.667 M; thus, carbicarb is also strongly hypertonic since its concentration of osmolites is far higher than the isotonic concentration of 300 mM. Interestingly, carbicarb was never adopted in clinical practice because it did not produce significantly better outcomes of treatment of states of acidemia in human patients beyond what the standard hypertonic solution of sodium bicarbonate produced. The reason for this clinical research result on humans was not fully understood, but it was clear that hypertonic carbicarb did not perform noticeably better than hypertonic sodium bicarbonate in a clinical setting. Thus, carbicarb was not found to be a more effective base-treatment solution for treating acidosis in a clinical setting than the standard treatment solution of sodium bicarbonate, and so it never replaced the standard treatment solution of sodium bicarbonate that is still commonly used today. Likewise, hypertonic solutions of pure Na₂CO₃ are strongly alkaline and can have deleterious effects if administered in any substantial quantity.

Hypertonic solutions containing osmolite concentrations much higher than isotonic, when added even in small relative volumes to blood, could adversely affect the structure and functionality of blood cells and other structures in the blood and surrounding tissues. So, it is possible that hypertonic base-treatment solutions have not been effective in treating human patients having acidemic states in prior studies because of adverse effects of such hypertonic solutions on blood cells and other structures. Destruction of even a fraction of a patient's blood cells by hypertonic base-treatment solutions could lead to additional unnecessary stress on the patient that would be highly undesirable.

Thus, there remains ample room for new innovations in the optimization of base-treatment solutions and in methods of administration of optimized base-treatment solutions that could substantially improve the treatment of acute metabolic acidosis beyond the current art.

### REFERENCES

Cooper D J, Walley K R, Wiggs B R and Russell J A. Bicarbonate does not improve hemodynamics in critically ill patients who have lactic acidosis. Ann Intern Med 112: 492-498, 1990.
Kraut, J. A. and Madias, N. E. Treatment of acute metabolic acidosis. Nephrol Nat Rev. 2012.
Kraut J A and Madias N E. Metabolic acidosis: pathophysiology, diagnosis and management. Nat Rev Nephrol 6: 274-285, 2010.
Leung J M, Landow L, Franks M, Soja-Strzepa D, Heard S O, Arieff A I and Mangano D T. Safety and efficacy of intravenous Carbicarb in patients undergoing surgery: comparison with sodium bicarbonate in the treatment of metabolic acidosis. Crit Care Med 22: 1540-1549, 1994.
Shapiro J I, Elkins N, Logan J, Ferstenberg L B and Repine J E. Effects of Sodium-Bicarbonate,
Disodium Carbonate, and A Sodium-Bicarbonate Carbonate Mixture on the P-CO2 of Blood in A Closed-System. Journal of Laboratory and Clinical Medicine 126: 65-69, 1995.
J. A. Kraut and I. Kurtz, Use of Base in the Treatment of Severe Acidemic States. Am J Kidney Diseases. 2001; 38: 703-727.
J. A. Kraut, Effect of Metabolic Acidosis on Progression of Chronic Kidney Disease. Am J Physiol Renal Physiol. 2011; 300: F828-F829.
G. F. Filley and N. B. Kindig, Carbicarb, an Alkalinizing Ion-Generating Agent of Possible Clinical Usefulness. Trans Am Clin Climatol Assoc. 1985; 96: 141-153.
R. M. Bersin and A. I. Arieff, Improved hemodynamic function during hypoxia with Carbicarb, a new agent for the management of acidosis. Circulation. 1988; 77:227-233.

In an embodiment of the current invention, a near-isotonic solution that includes a strong base in its composition is administered to an acidemic patient to raise the patient's blood pH, lower the patient's blood PCO2, and minimize damage to the patient's blood cells, thereby effectively treating the patient's acidemia. This approach differs from the prior art because we use a lower, near-isotonic concentration of a solution that contains a strong base, and this provides a clear improvement over high concentration solutions that have been used in the past. In particular, we find that near-isotonic solutions that contain strong bases, including but not limited to disodium carbonate, sodium hydroxide, and tris(hydroxymethyl)aminomethane (Tris), can be used to raise blood pH into a desirable range, lower blood PCO2 into a desirable range, and minimize visible damage to blood cells as probed using optical microscopy. Because we use higher concentrations of a strong base than in carbicarb, and because we limit the concentration of total osmolite species in the base-treatment solution to be much lower, in the near-isotonic range, we achieve significantly improved performance in treating acidemia over the prior art. We also show that the two high concentration treatment solutions of standard 1.0 M sodium bicarbonate and carbicarb of the prior art lead to significant damage to blood cells because both of these solutions have high osmolite concentrations compared to isotonic. This potentially explains the adverse effects and lack of improvement in clinical outcomes of standard sodium bicarbonate solutions as well as carbicarb solutions in clinically treating acidemia in patients.

In another embodiment of the current invention, one or more near-isotonic base-treatment solutions, at least one solution of which contains a strong base, are administered to a patient having an acidemic state using a computer-controlled system that includes a computer and a computer-controllable liquid dispensing system which uses real-time and stored changes in measured blood parameters of a patient, read from a computer-connected device, including but not limited to a pH meter and an optical blood-gas analyzer, in a feedback loop to optimize the administration of said near-isotonic base-treatment solution to said patient having an acidemic state.

In another embodiment of the current invention, a composition of a base-treatment solution for treating acidosis is an aqueous solution of Na₂CO₃:NaHCO₃ at a molar ratio of about 2:1 or larger, wherein the molar ratio is selected to achieve both a desired pH increase and a desired PCO2 reduction, and wherein the total concentration of osmolites in said base-treatment solution is in a range that is near-isotonic.

In another embodiment of the current invention, a composition of a base-treatment solution for treating acidosis is an aqueous solution of NaOH:NaHCO₃ at a molar ratio of about 2:1 or larger, wherein the molar ratio is selected to achieve both a desired pH increase and a desired PCO2 reduction, and wherein the total concentration of osmolites in said base-treatment solution is in a range that is near-isotonic.

In another embodiment of the current invention, a composition of a base-treatment solution for treating acidosis is an aqueous solution of NaOH:Na₂CO₃, wherein the concentrations of NaOH and Na₂CO₃, the molar ratio, and the total solution volume are selected to achieve a desired pH increase, a desired PCO2 reduction, and a desired concentration of bicarbonate ion [HCO₃⁻], and wherein the total concentration of osmolites in said base-treatment solution is in a range that is near-isotonic.

### Experimental Protocol

We have designed and carried out experiments using canine blood that is more acid than normal to measure how new types of base-treatment solutions affect blood chemistry in order to optimize the composition and concentration of the base-treatment solution for treating acidosis.

The primary blood chemistry tests were carried out using an IDEXX VetStat Blood Gas Analyzer (BGA) and single-use IDEXX Electrolyte 8+ cassettes. The VetStat BGA was calibrated using all standard reference cassettes, passing the necessary reference tests, and it was also calibrated using Levels 1, 2, and 3 OptiCheck fluids, passing all calibration fluid tests successfully. The BGA directly measures pH and PCO2 and uses these values to determine the concentration of the bicarbonate ion HCO₃⁻. The pH range of the BGA has a lower limit of 6.4 and an upper limit of 7.8, and the PCO2 has a lower limit of 10 mm Hg. The BGA also directly measures [Na⁺], [K⁺], and [Cl⁻]. All BGA measurements are made at a temperature T=37° C. In addition, simple pH measurements were made using an Accumet pH meter equipped with an electrode probe suitable for small volumes. Optical microscopy was performed with a National Optical brightfield microscope using a 40× objective and a digital camera (Flea2 by Point Grey Research). The image size (i.e. distance scale) has been calibrated using a 100 line-per-mm reticle. Simple pH measurements using the meter and optical microscopy were carried out at room temperature T 23° C.

Canine blood was obtained from Animal Blood Resources International (Dixon, Calif.) and stored at 4° C. This blood has been treated with a citrate-type anticoagulant; this type of anticoagulant, which can contain citric acid, can lower blood pH from normal. The first blood sample is “canine blood of 7 May 2013” (measured pH=6.98), more acidic than normal, received fresh from the supplier on 7 May 2013. The second blood sample is “canine blood of 9 May 2013” (measured pH=6.90), slightly more acidic because aging of the blood during storage over two days occurred. The third blood sample is “HCl-acidified canine blood of 9 May 2013” (measured pH=6.62), in which 1 part of a 150 mM solution of hydrochloric acid (HCl) was added to 9 parts of canine blood of 9 May 2013. The fourth blood sample is “canine blood of 21 May 2013” (measured pH=7.00) which was received fresh from the supplier in a separate shipment on 21 May 2013. The fifth blood sample is “HCl-acidified canine blood of 21 May 2013” (measured pH=6.66), in which 1 part of a 150 mM solution of HCl was added to 9 parts of canine blood of 21 May 2013.

Blood samples were treated with base-treatment solutions by adding 1 part of the base-treatment solution (e.g. 100 microliters) to 9 parts of canine blood (e.g. 900 microliters) and mildly agitating for 10-20 seconds. The base-treated blood was then injected using a syringe into a heparin-treated capillary tube of 200 microliter capacity. The capillary tube was inserted into the aspiration nozzle of the Electrolyte 8+ cassette of the VetStat BOA, and the BOA then tested the blood sample and recorded the results. The entire procedure takes less than five minutes from mixing to loading of the BGA. A small amount of the base-treated blood was transferred onto a glass slide with a No. 1 coverslip for viewing using the optical microscope.

We use several conventions for describing the aqueous base-treatment solutions that we have prepared. Compositions and concentrations of base-treatment solutions containing carbonate species refer to the concentration carbonate species. These solutions have been made by dissolving solid bases or hydrates of bases into de-ionized water. For instance, in an aqueous solution of 150 mM NaHCO₃, the sodium bicarbonate dissociates into 150 mM HCO₃⁻ and 150 mM Na⁺. Likewise, in an aqueous solution of 150 mM Na₂CO₃, disodium carbonate dissociates into 150 mM CO₃²⁻ and 300 mM Na⁺. In the case of a mixture of two different carbonate base types, we specify individual molarity of each base. For example, 50 mM NaHCO₃: 100 mM Na₂CO₃ refers to an aqueous solution that has a total dissolved concentration of all carbonate ionic species of 150 mM and a total concentration of dissolved Na⁺ of 250 mM. For mixtures of two different strong base types, regardless of whether or not they are both carbonates, such as Na₂CO₃ and NaOH, we specify the individual molarity of each base and the total molarity of strong base ions. For example 75 mM Na₂CO₃: 75 mM NaOH refers to an aqueous solution that has a total strong base ion concentration of 150 mM, since both CO₃²⁻ and OH⁻ are strong bases, and a total concentration of dissolved Na⁺ of 225 mM.

We assume that a total ionic concentration (combined concentrations of all positive and negative ions that function osmotically, excluding H₃O⁺ and OH⁻) in blood is about 300 mM, so we assume that 300 mM is isotonic, and total ionic concentrations that lie within a range from about 150 mM to about 450 mM are near-isotonic. Many of the base-treatment solutions that we test are at or near this value of total ionic concentration, so we refer to such solutions as isotonic or near-isotonic. For example, 150 mM NaHCO₃ yields a total ionic concentration of 300 mM after dissociation in water ([Na+]=150 mM and [HCO₃⁻]=150 mM), so this is quite close to isotonic. As a different example, 150 mM Na₂CO₃ yields a total ionic concentration of 450 mM because [Na⁺]=300 mM and [CO₃²⁻]=150 mM, which, although somewhat larger than 300 mM, we still consider to be a near-isotonic base-treatment solution.

In some base-treatment solutions, we have added sodium chloride NaCl in order to raise the concentration of effective osmolites to near-isotonic. For instance, 150 mM NaOH solution only has 150 mM of osmotically effective species after dissociation: 150 mM [Na⁺]; the 150 mM [OH⁻] is ineffective as an osmotic agent. However, we have also made a 150 mM NaOH: 75 mM NaCl solution; this solution is near-isotonic because the total concentration of effective osmolites is near 300 mM (i.e. [Na⁺]=225 mM and [Cl⁻]=75 mM). Thus, we recognize that 150 mM NaOH: 75 mM NaCl solution is much closer to isotonic, yet we still refer to 150 mM NaOH solution as near-isotonic.

### Results

The results of BGA tests have been organized into Tables I-IV. Table I summarizes results for 150 mM strong base or HCl strong acid solutions added to canine blood of 9 May 2013. Strong bases raise pH substantially, and the strong acid HCl lowers pH. Table II summarizes results of adding 150 mM base-treatment solutions to Ha-acidified canine blood of 9 May 2013. Sodium bicarbonate raises the pH only by about 0.2 pH units, and it elevates PCO2 by about 28 mm Hg. By contrast, base-treatment solutions of strong bases, such as Na₂CO₃ and NaOH, raise pH by about 0.5 pH units. We find that Na₂CO₃ is highly effective in raising pH, while lowering PCO2 substantially, and contributing a positive increase to [HCO₃-]. We find that NaOH, which is a strong base yet contains no carbonate species, is also highly effective in raising pH. However, the 150 mM base-treatment solution of NaOH lowers PCO2 even more than the solution of Na₂CO₃, so solutions of NaOH cause very little change, perhaps even a small reduction, in [HCO₃⁻]. We also find that a mixed base-treatment solution that is 75 mM Na₂CO₃: 75 mM NaOH, which has 150 mM total of strong base species, raises pH by about the same amount as either 150 mM pure Na₂CO₃ or 150 mM pure NaOH solutions, yet has values of PCO2 and [HCO₃⁻] that are intermediate between the two pure solutions. Thus, by controlling the proportion of Na₂CO₃ and NaOH in the solution, yet keeping the total ion concentration in the near-isotonic range, it is possible to control the amount of change in pH and reduction in PCO2 independently. In Table III, we summarize results for equimolar mixtures of sodium bicarbonate and disodium carbonate base-treatment solutions added to HCl-acidified canine blood of 9 May 2013. The solution at 333 mM NaHCO₃: 333 mM Na₂CO₃ is standard carbicarb. As such, carbicarb lies far outside of the near-isotonic range, so carbicarb is not a near-isotonic base-treatment solution. The measured pH increase is about 1.7 (beyond the range of the BGA but measured using the pH meter) and reduces PCO3 to about 13 mm Hg. In Table IV, we summarize responses of canine blood of 7 May 2013 treated using varying proportions of sodium bicarbonate and disodium carbonate base-treatment solutions at near-isotonic concentrations. By mixing different proportions of Na₂CO₃ and NaHCO₃ into the near-isotonic treatment solution, the increase in pH and reduction in PCO2 can both be controlled independently. In Table V, we summarize responses of canine blood of 21 May 2013 treated using various base-treatment solutions that are near-isotonic. In Table VI, we summarize responses of HCl-acidified canine blood of 21 May 2013 treated using various base-treatment solutions that are near-isotonic. These responses are very similar to the prior results and demonstrate good reproducibility of our measurements. In Tables V and VI, we include results for Tris buffer at pH=8.1, as well as Tris solutions at higher pH than the buffer, and we also include results for base-treatment solutions that contain added saline in order to raise the total concentration of osmolites to near-isotonic. Table VII reports the measured pH of the base-treatment solutions used.

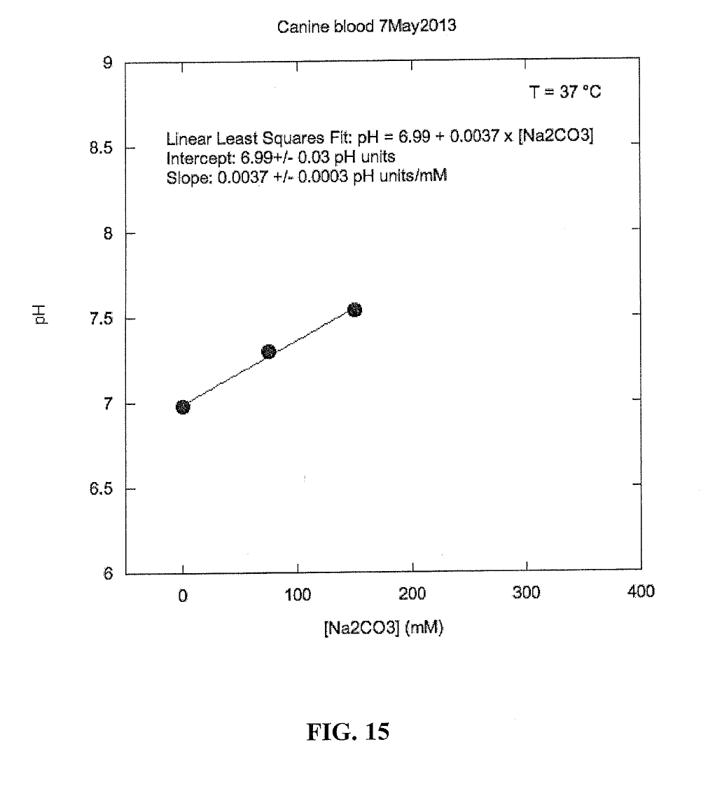

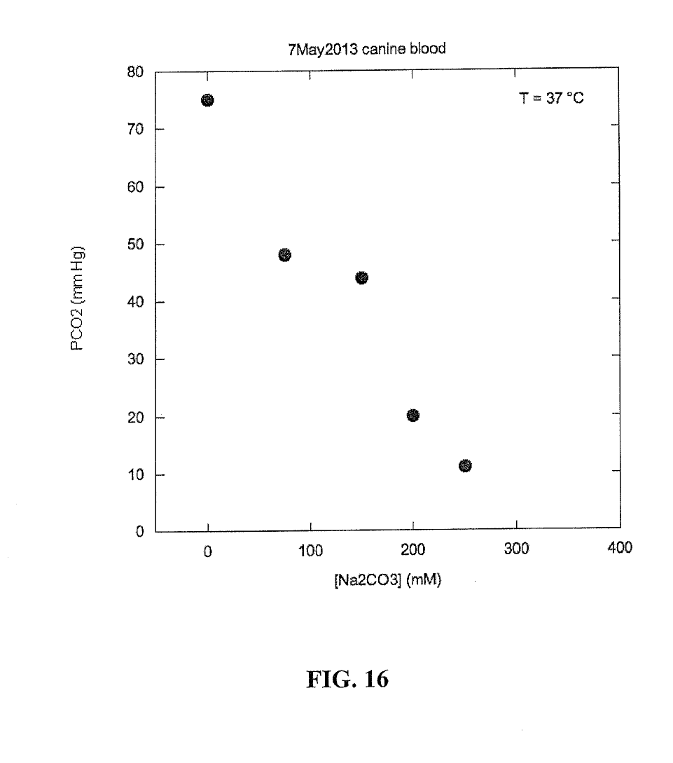

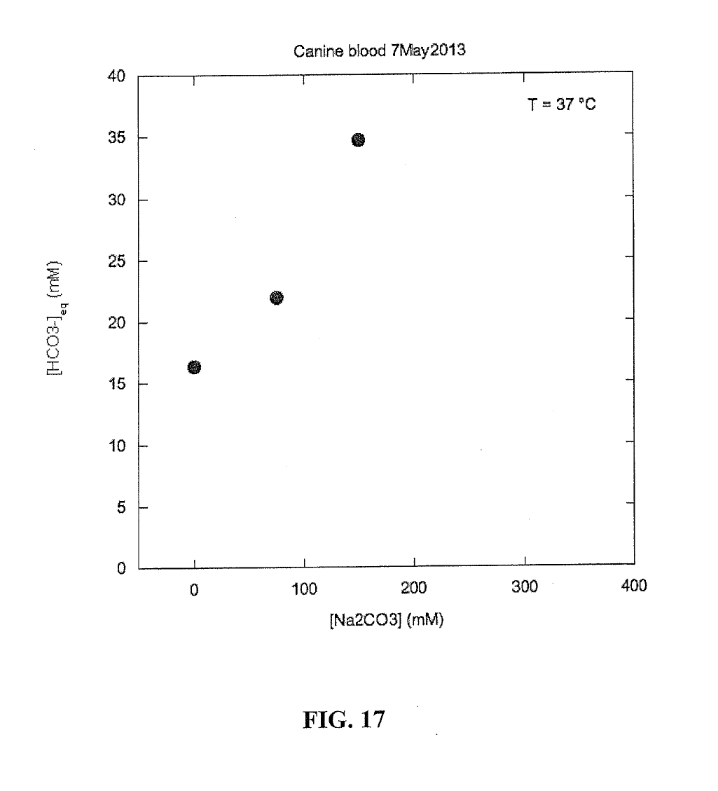

FIGS. 15-17 show BGA results for pH, PCO2, and [HCO₃⁻]_{eq} respectively after disodium carbonate base-treatment solutions having concentrations below, near, and above 150 mM carbonate were added to canine blood of 7 May 2013. The pH is raised, PCO2 is lowered, and [HCO₃⁻] is raised.

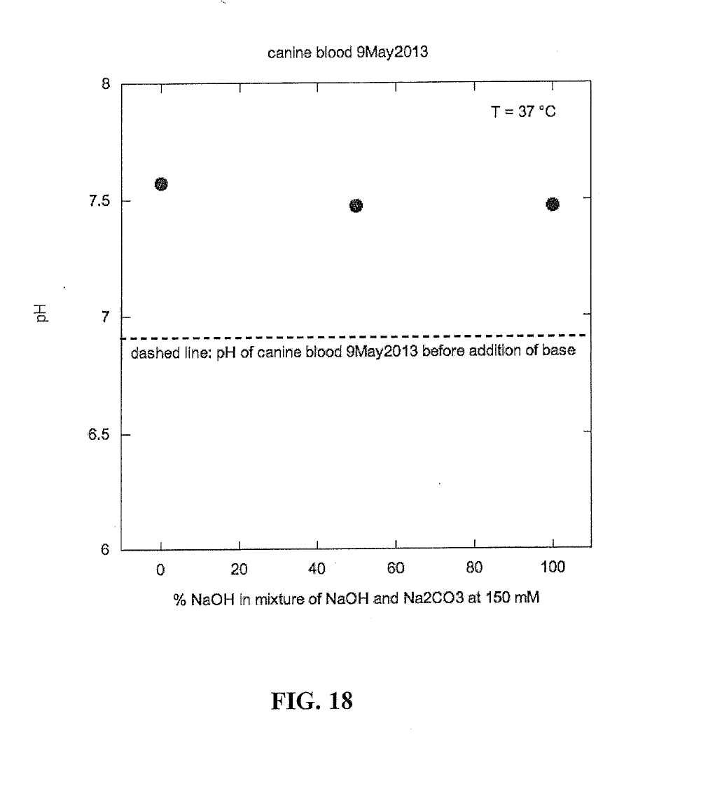

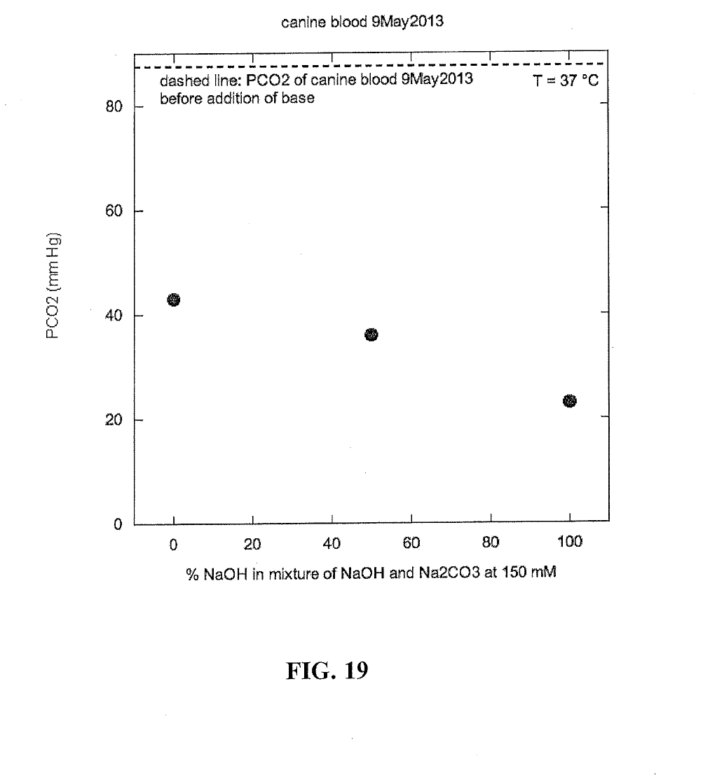

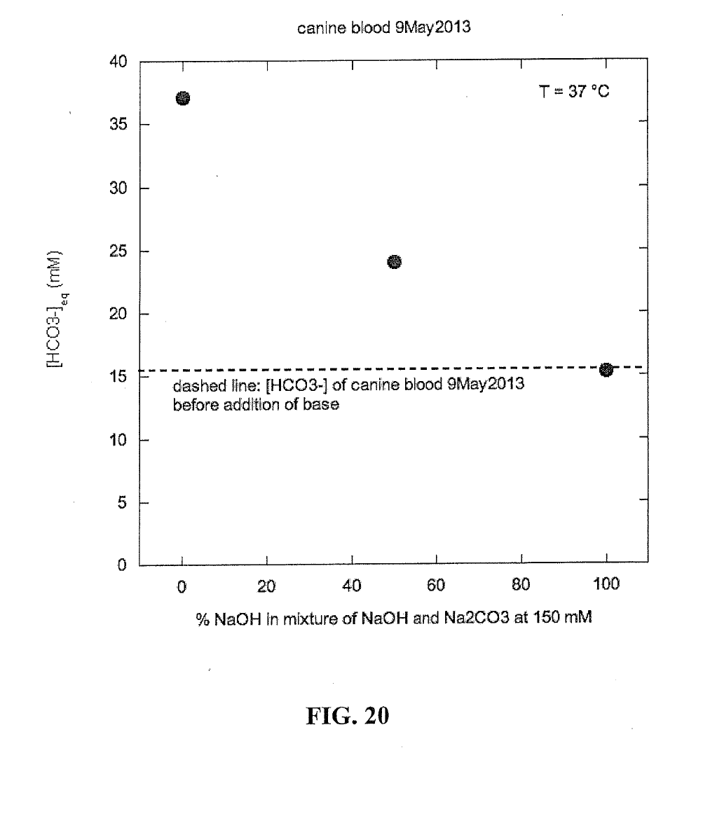

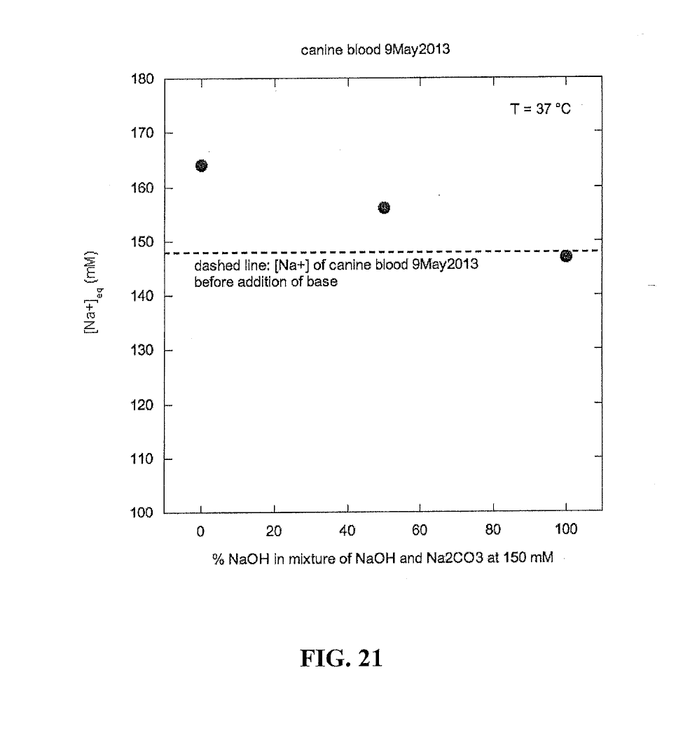

FIGS. 18-21 show BGA results for pH, PCO2, [HCO₃⁻]_{eq}, and [Na⁺]_{eq} respectively after near-isotonic 150 mM base-treatment solutions containing NaOH only, Na₂CO₃ only, and 50% NaOH mixed with 50% Na₂CO₃ were added to canine blood of 9 May 2013. All three base-treatment solutions provide about the same increase in pH, but PCO2 is more substantially reduced by using a base-treatment solution that contains more NaOH than Na₂CO₃ in the mixture. While NaOH provides essentially the same pH reduction as Na₂CO₃, the equilibrium sodium ion concentration in the treated blood is lower for NaOH than for Na₂CO₂ because NaOH is monovalent in sodium but Na₂CO₃ is divalent in sodium.

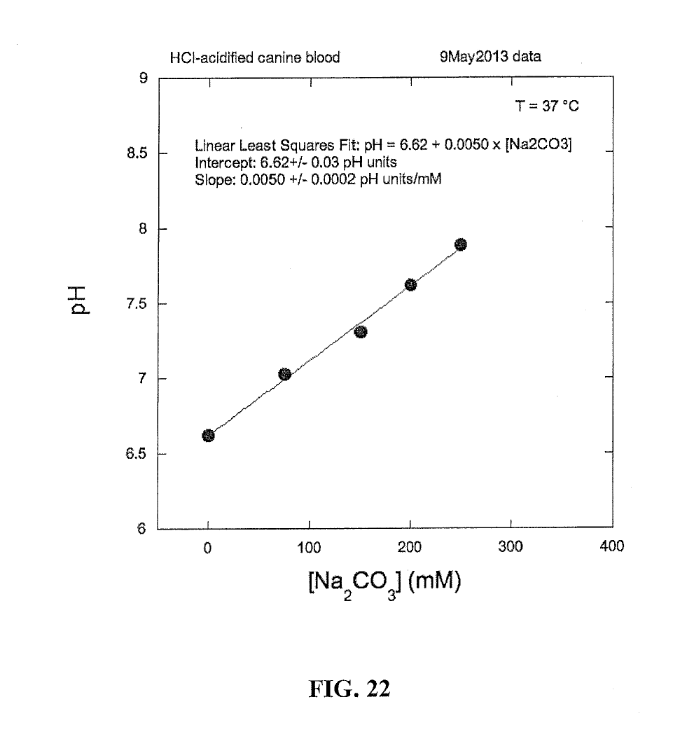

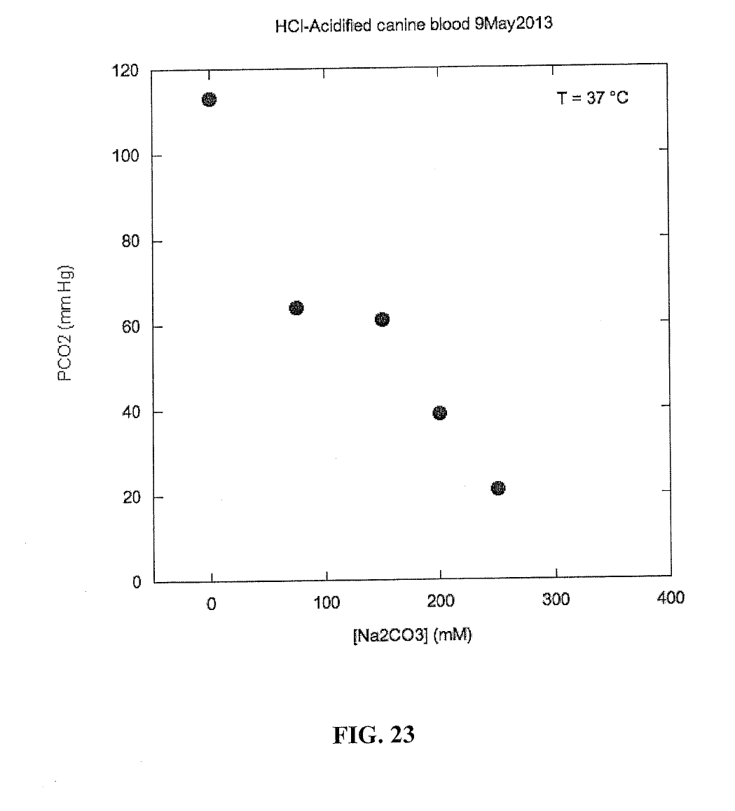

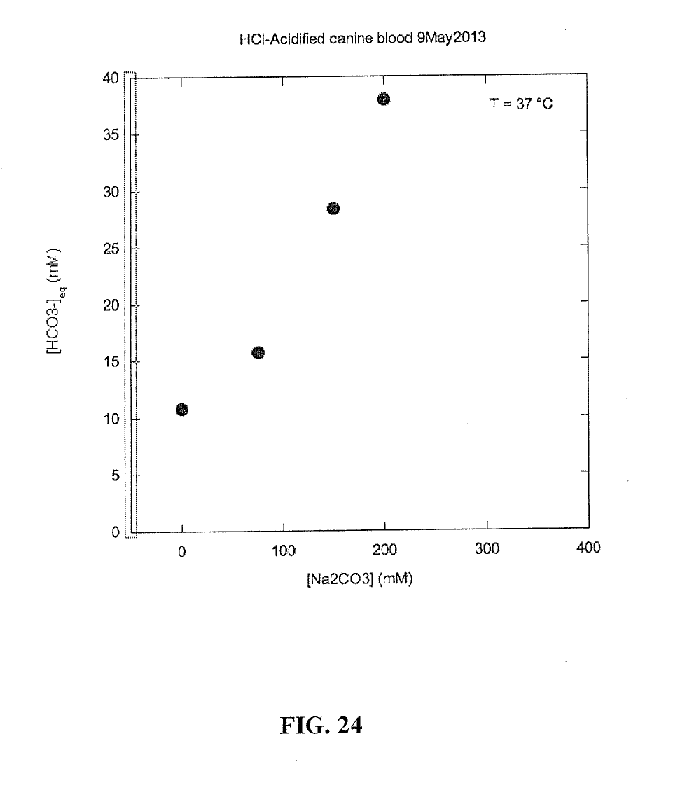

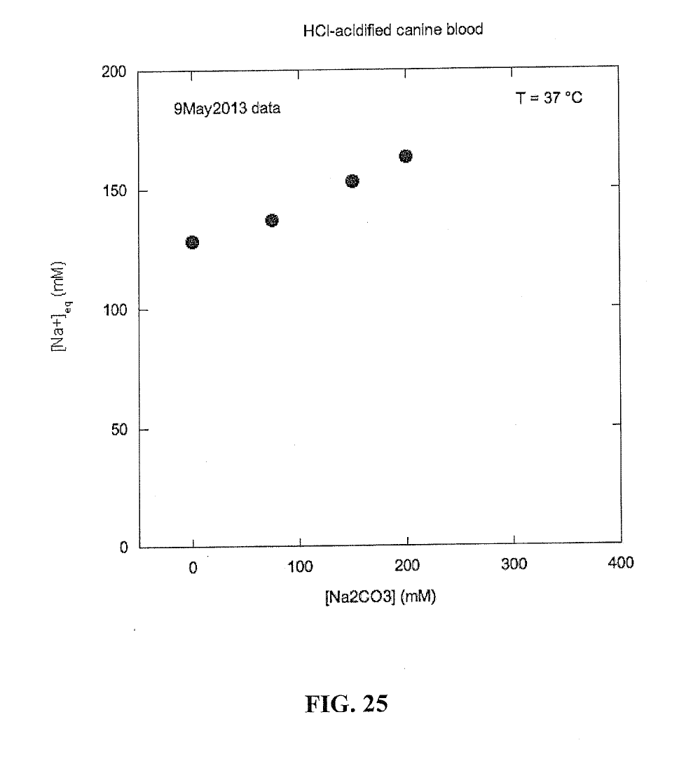

FIGS. 22-25 show BGA results for pH, PCO2, [HCO₃⁻]_{eq}, and [Na⁺]_{eq} respectively after the addition of disodium carbonate base-treatment solutions at different concentrations to HCl-acidified canine blood of 9 May 2013. Although the starting pH of the blood prior to the addition of the base-treatment solution is lower, the trends in the measured quantities are very similar to those in FIGS. 1-3. The lower starting pH enables more measurements to be made with the BGA, since the pH after treatment is better adapted for the range of the BGA.

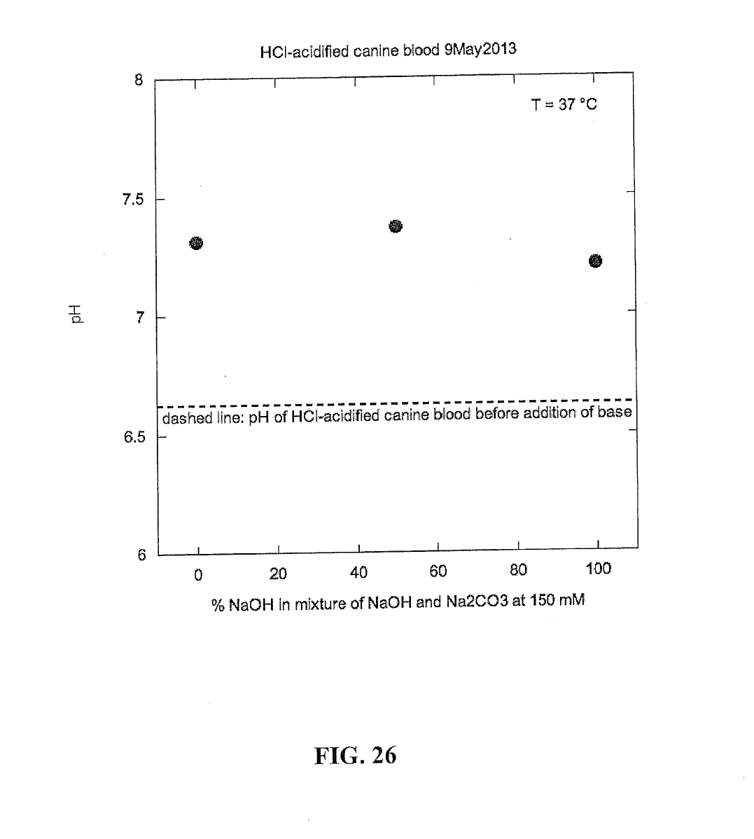

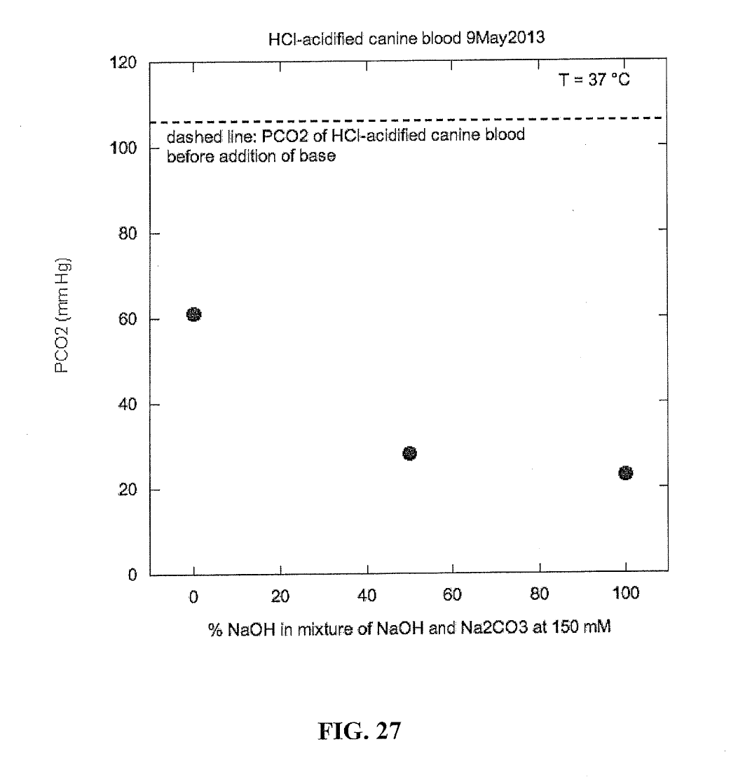

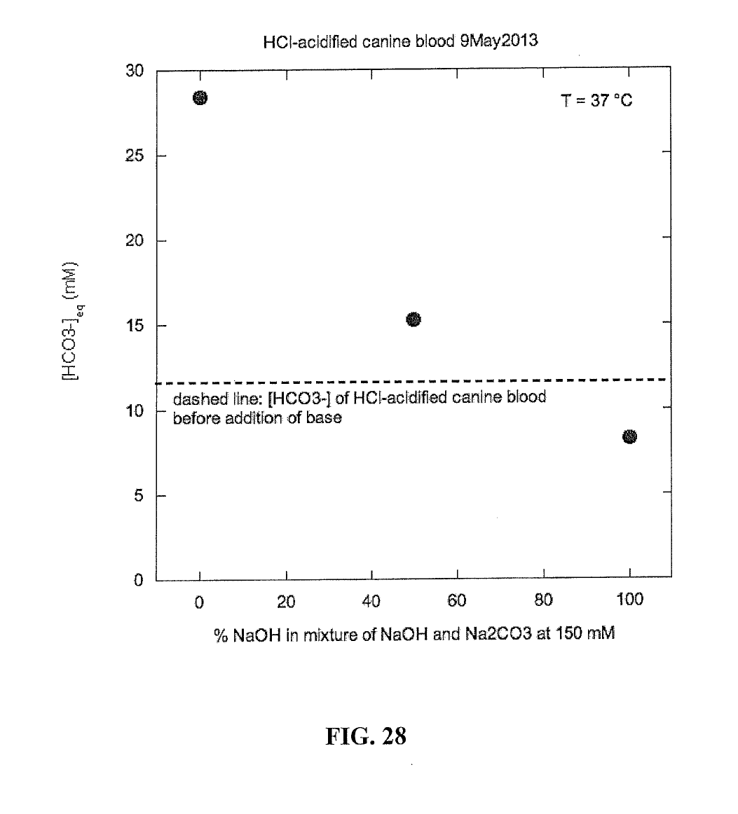

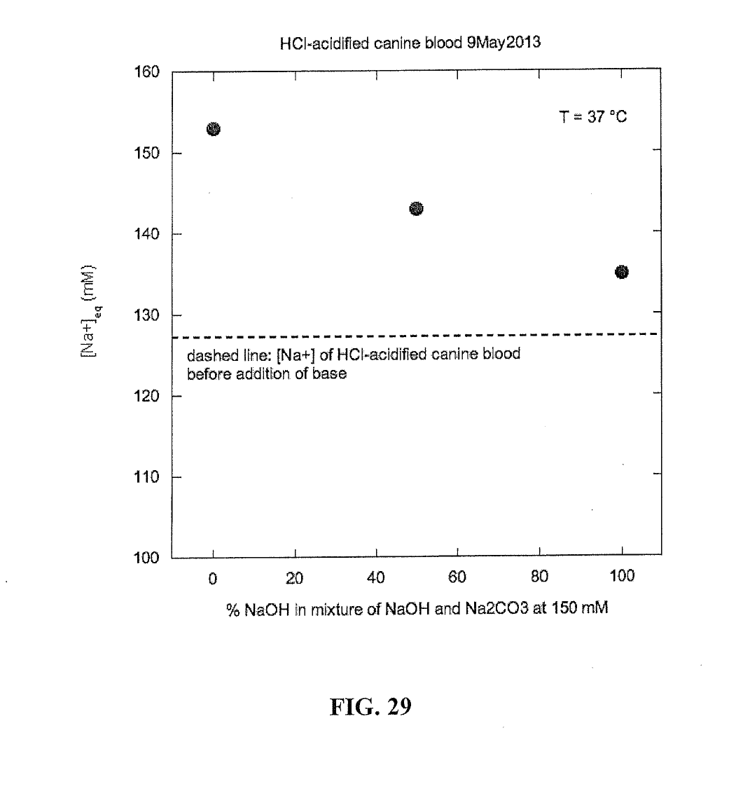

FIGS. 26-29 show BGA results for pH, PCO2, [HCO₃⁻]_{eq}, and [Na⁺]_{eq} respectively after near-isotonic 150 mM base-treatment solutions containing NaOH only, Na₂CO₃ only, and 50% NaOH mixed with 50% Na₂CO₃ were added to HCl-treated canine blood of 9 May 2013. The trends in the results are very similar to those in FIGS. 18-21 even if the starting pH was lower because of HCl-acidification of the blood.

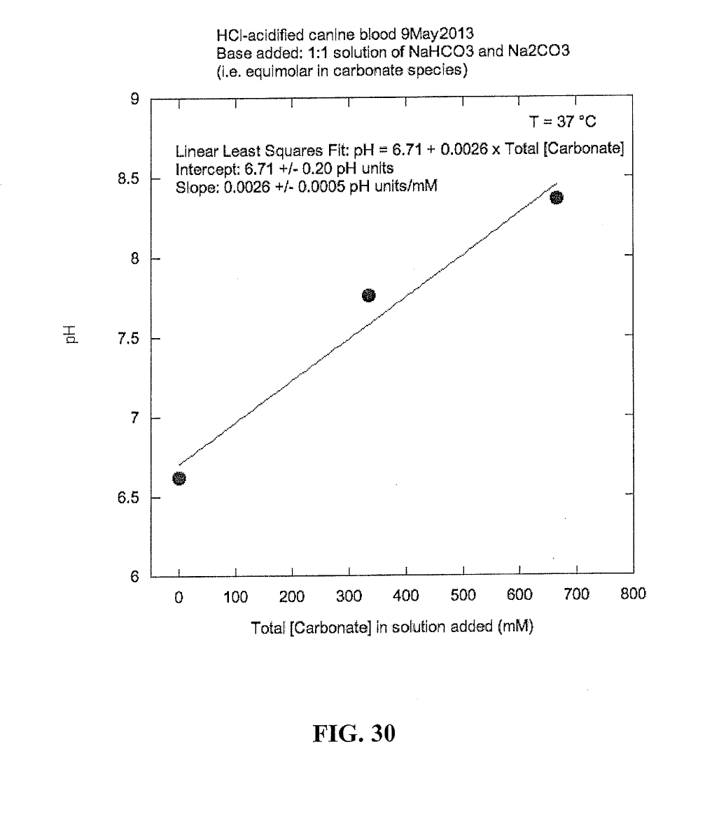

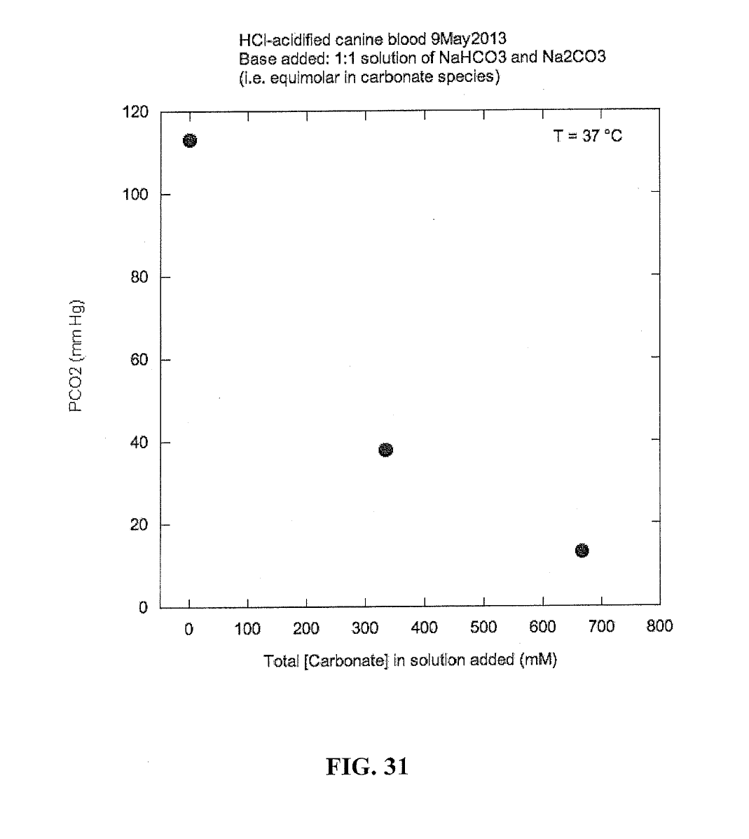

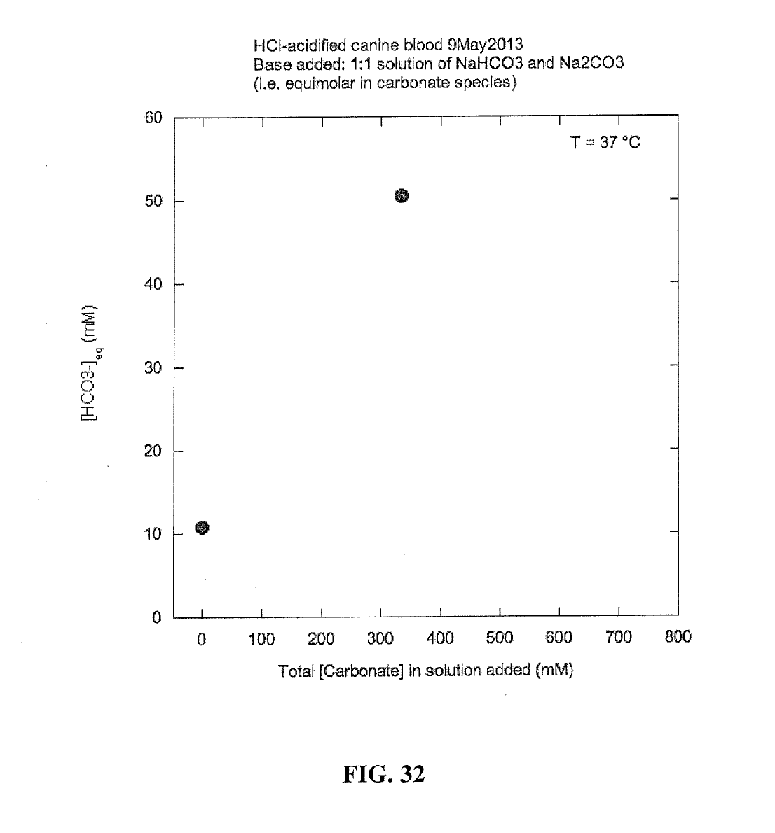

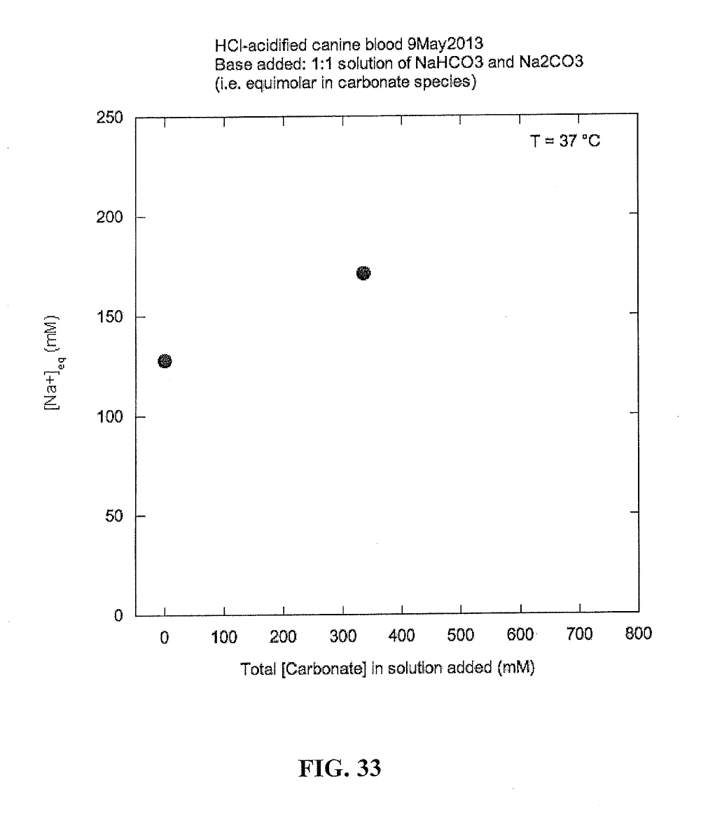

FIGS. 30-33 show BGA results for pH, PCO2, [HCO₃⁻]_{eq}, and [Na⁺]_{eq} respectively after equimolar base-treatment solutions, which contain equal proportions of NaHCO₃ to Na₂CO₃ but different total carbonate species concentrations, were added to HCl-treated canine blood of 9 May 2013. Carbicarb corresponds to a total carbonate concentration of 667 mM and a total ionic concentration of 1.0 M, far above the isotonic level.

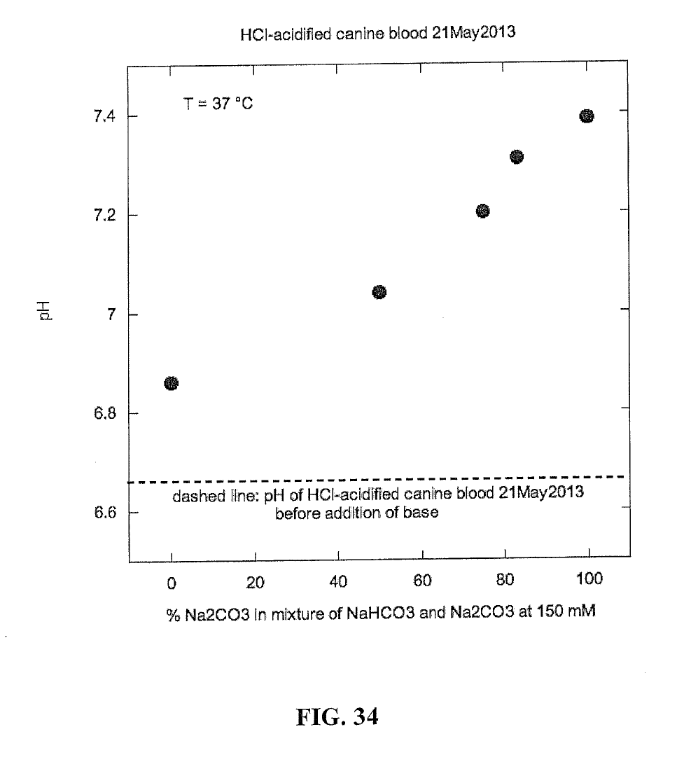

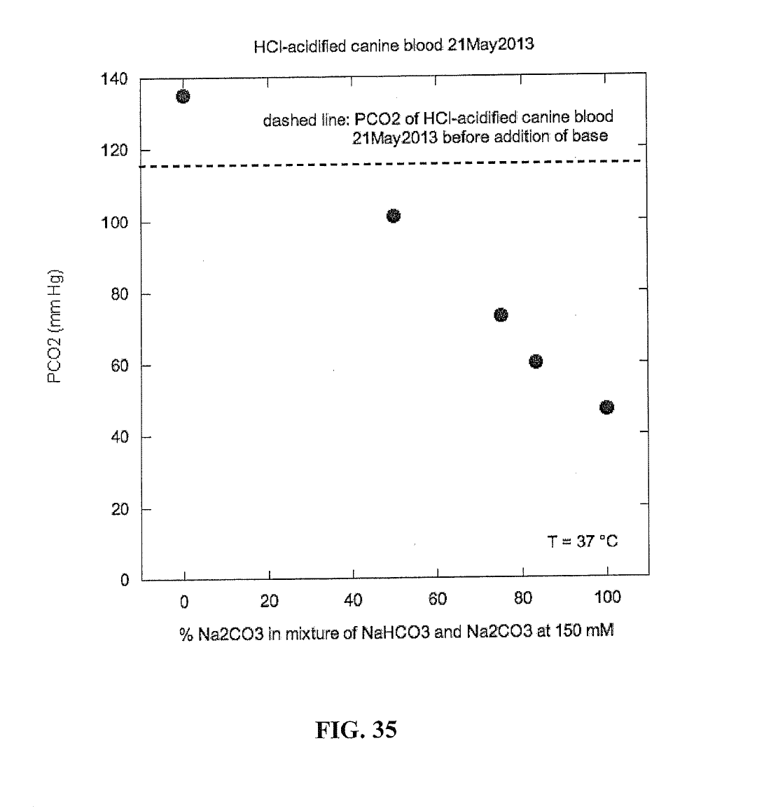

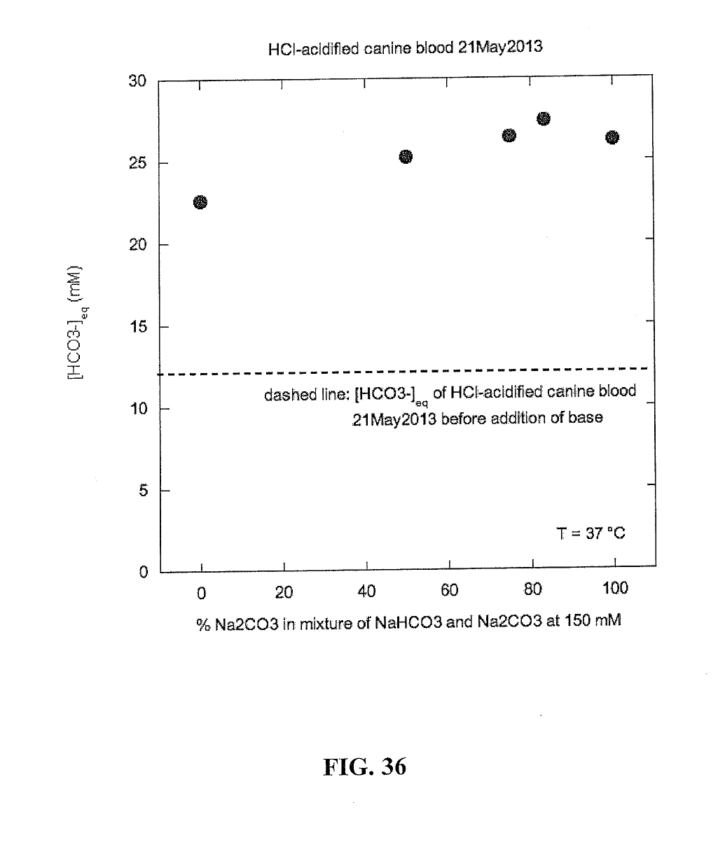

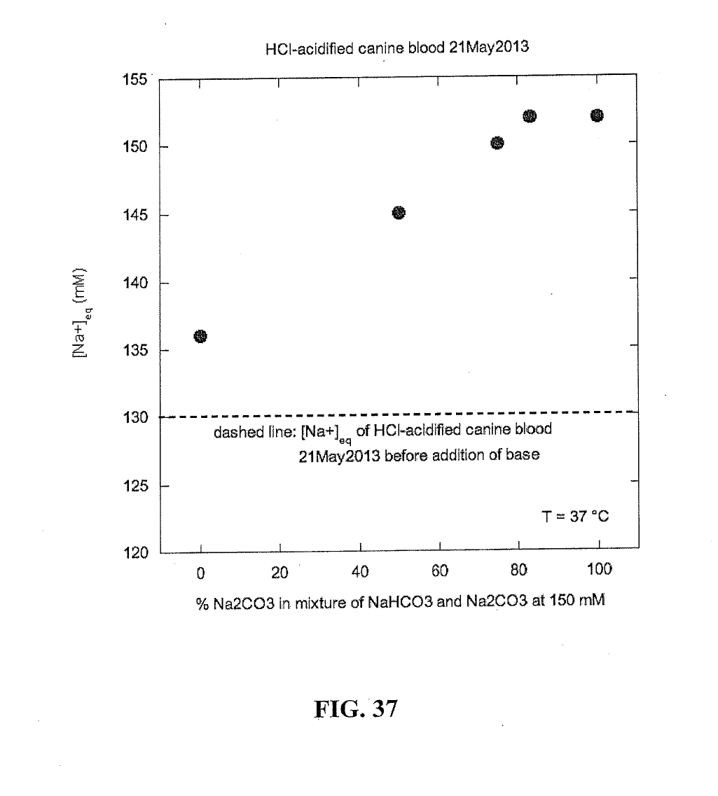

FIGS. 34-37 show BGA results for pH, PCO2, [HCO₃⁻]_{eq}, and [Na⁺]_{eq} respectively after base-treatment solutions, which contain differing proportions of NaHCO₃ to Na₂CO₃ but a fixed total added carbonate species concentration of 150 mM, were added to HCl-treated canine blood of 21 May 2013. These results show that as a larger percentage of Na₂CO₃ is used in the mixed base-treatment solution, the efficacy in simultaneously raising pH and lowering PCO2, both of which can be desired in the treatment of acidosis, of the treated blood is increased. Moreover, the bicarbonate ion concentration can be replenished while raising pH and lowering PCO2 by different controllable amounts.

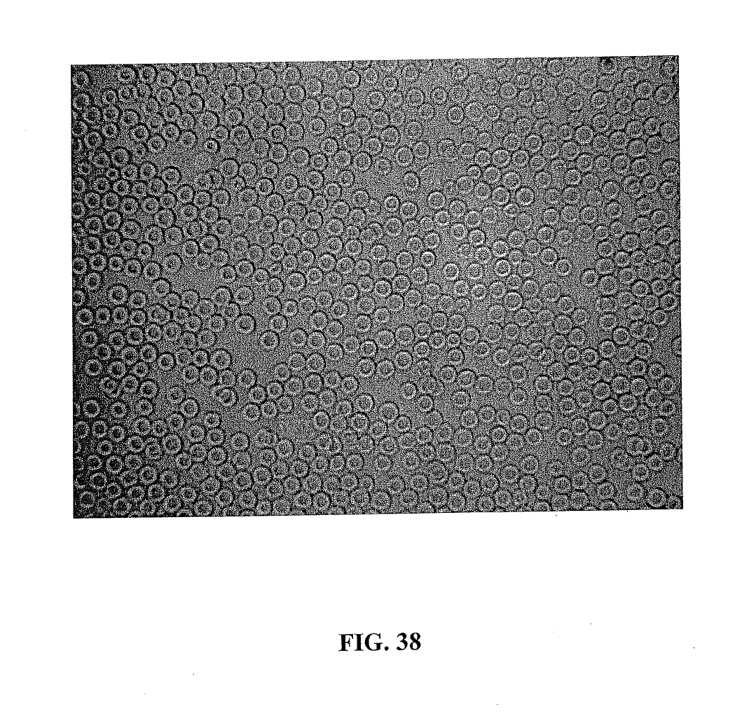

FIGS. 38-45 show the results of optical microscopy of canine blood, both untreated and treated. The images shown are representative of the entire sample, not singled out for peculiar features.

FIG. 38 shows an example of the appearance of untreated canine blood; red blood cells (RBCs) appear biconcave and few if any are spiculated (i.e. spiky), broken, or deformed. Rouleaux, columnar aggregates of RBCs, are not observed. RBCs have diameters that are approximately 7 microns, consistent with prior reports. The outermost diameter may appear to be somewhat larger than this value due to diffraction effects in the microscopy.

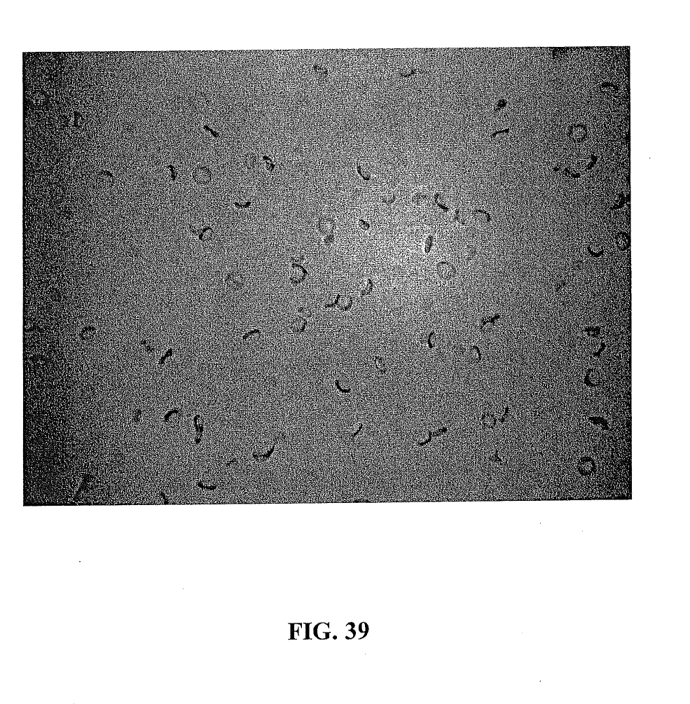

FIG. 39 shows an example of the appearance of canine blood treated with 1.0 M NaHCO₃, a standard base-treatment solution of sodium bicarbonate that is commonly used in medical practice to treat acute metabolic acidosis. Considerable damage is caused to RBCs as a result of using this treatment solution. Based on this finding, in the region where the 1.0 M NaHCO₃ first contacts the blood (e.g. if administered intravenously as it exits the injection point and enters the blood), it would be likely that at least some RBCs would be damaged. Thus, this standard treatment solution has a concentration far above isotonic, and this treatment solution can cause damage to blood even if sodium bicarbonate is a weak base having a pKa that is not far above the normal pH of blood.

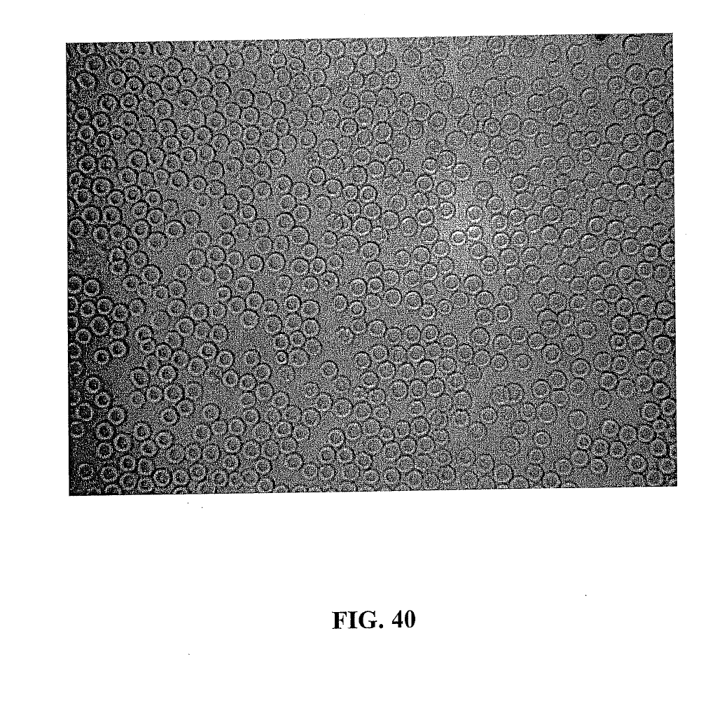

FIG. 40 shows an example of the appearance of canine blood that has been treated with a near-isotonic base-treatment solution of 150 mM Na₂CO₃. The RBCs are clearly biconvex and are not spiculated; the RBCs appear very similar to those in FIG. 38, so the RBCs are thus essentially undamaged, at least by visual inspection, by the base-treatment process. This shows that a near-isotonic base-treatment solution of a strong base, in this case disodium carbonate, can be mixed into blood without causing visible damage to RBCs. Because the near-isotonic solution of strong base 150 mM Na₂CO₃ is effective in raising pH and lowering PCO2 in blood, plus it does not visibly damage RBCs, it thus appears to be closer to optimal as a base-treatment solution than highly concentrated solutions that contain a weak base, such as the commonly used 1.0 M NaHCO₃.

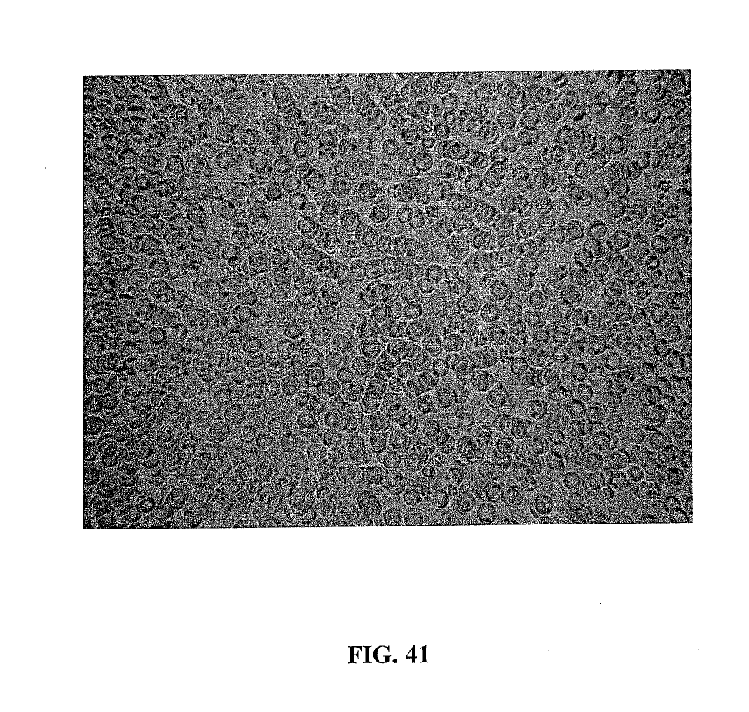

FIG. 41 shows an example of the appearance of canine blood that has been treated with a near-isotonic base-treatment solution of 150 mM NaOH. Relatively little damage to RBCs is observed; a few spiculated RBCs are seen (less than 10% of the population). However, rouleaux formation is evident. Spiculation or rupturing of RBCs are much more serious types of blood damage. In some species, removed blood that is not flowing in blood vessels exhibits rouleaux formation (e.g. in equine blood); thus, blood can still be functional and viable even if rouleaux are seen. So, the observation of rouleaux formation of RBCs, while not ideal, is of minor importance compared to changes in the shape and integrity of RBCs.

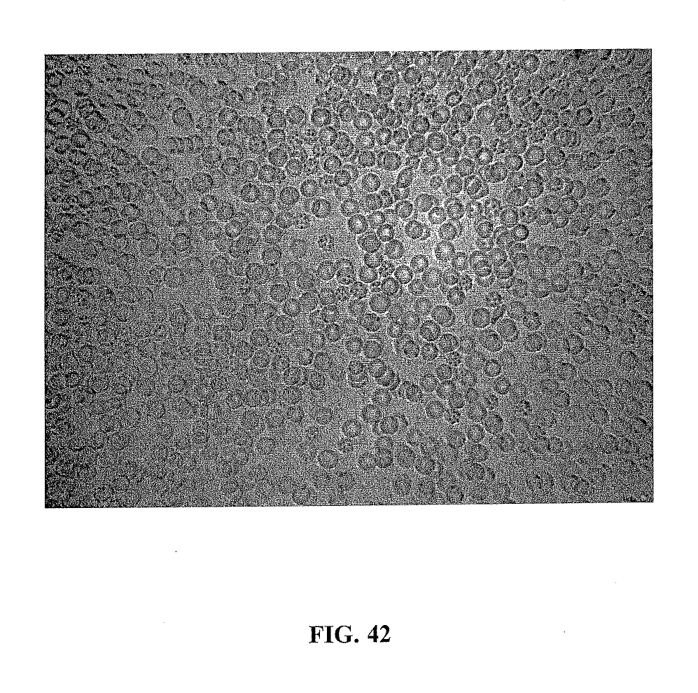

FIG. 42 shows an example of the appearance of canine blood that has been treated with a near-isotonic base-treatment solution of 75 mM NaOH:75 mM Na₂CO₃, yielding a total concentration of strong base species of 150 mM (i.e. having a similar power to raise pH as solutions in FIG. 40 and FIG. 41). Although a very small population (less than 10%) of spiculated RBCs are observed, the vast majority of RBCs are undamaged, similar to FIG. 41. Rouleaux formation is largely absent in FIG. 42; only a few very small aggregates are seen.

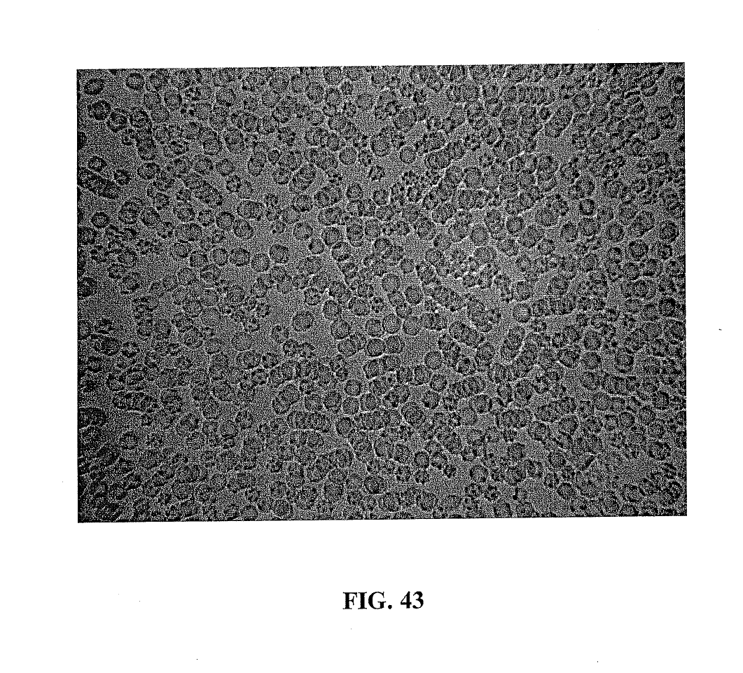

FIG. 43 shows an example of the appearance of canine blood that has been treated with 150 mM HCl. A significant population (approximately 30%) of the RBCs exhibit spiculation, and rouleaux are also observed. These features make the interpretation of microscope images of HCl-acidified blood that has been subsequently treated with base-treatment solutions very difficult. Presumably, spiculation and other forms of damage to the shape and integrity of RBCs are irreversible, so administration of a base might not cause a return of the spiculated cells to a biconcave shape. Consequently, we do not show any images of base-treated blood that has been acidified using HCl, since these images contain at least as many spiculated RBCs as we have observed in HCl-acidified blood of 9 May 2013.

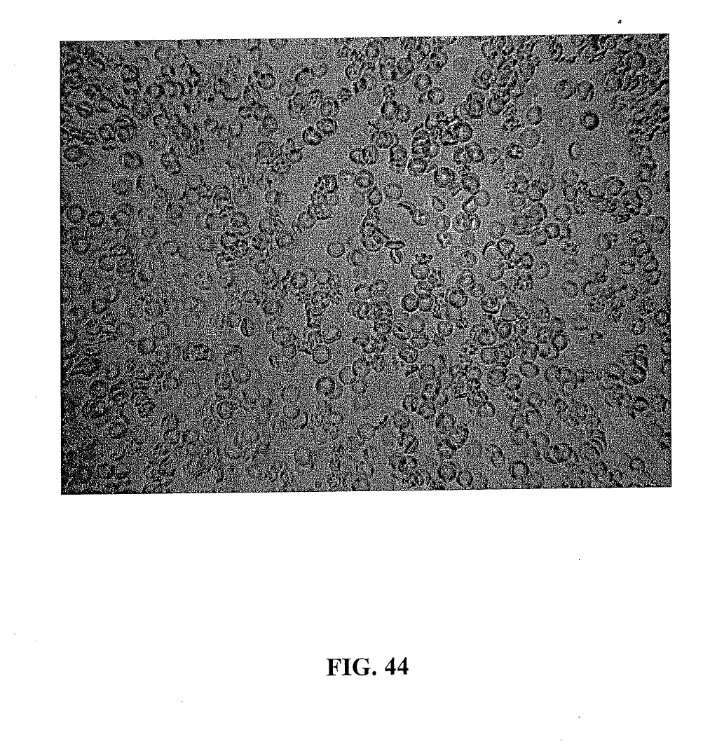

FIG. 44 shows an example of the appearance of canine blood that has been treated with carbicarb, 333 mM NaHCO₃: 333 mM Na₂CO₃, yielding a total bicarbonate concentration of 0.667 M, far above isotonic. A large fraction, nearing about 50%, of RBCs are damaged: noticeably spiculated, deformed, or broken. Some smaller rouleaux are also observed of the remaining biconcave fraction of RBCs. This micrograph, combined with FIG. 39, provides evidence that highly concentrated solutions (far above isotonic) that contain weak bases, such as NaHCO₃ are far from optimal base-treatment solutions for treating acute metabolic acidosis.

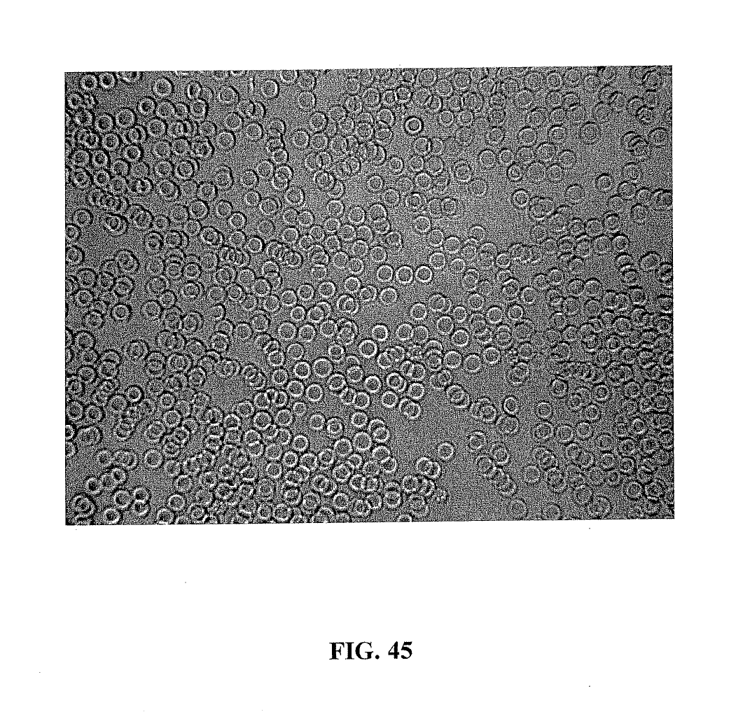

FIG. 45 shows an example of the appearance of canine blood of 21 May 2013 treated with a near-isotonic solution of saline-supplemented sodium hydroxide (1 part 150 mM aqueous solution of NaOH containing 75 mM NaCl added to 9 parts blood). Nearly all RBCs are biconcave (i.e. normal) in shape and very few are spiculated. However, a few smaller rouleaux are present. Overall, the addition of a small saline concentration appears to reduce the amount of spiculation and rouleaux formation compared to 150 mM NaOH only treatment shown in FIG. 41.

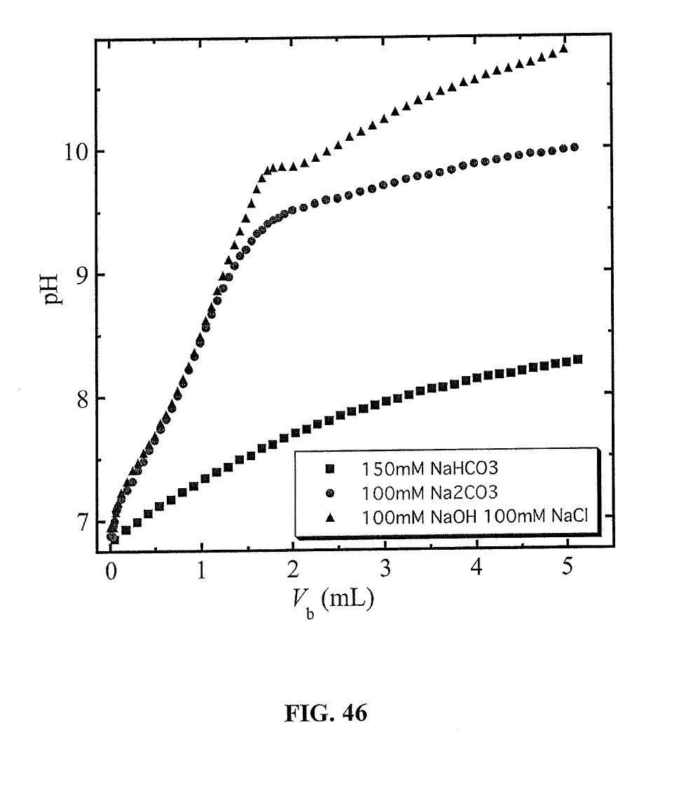

FIG. 46 shows pH measurements during titration of HCl-acidified canine blood using three different near-isotonic base-treatment solutions: 150 mM NaHCO₃, 100 mM Na₂CO₃, and 100 mM NaOH: 100 mM NaCl. The initial volume of HCl-acidified canine blood is 4.0 mL; the volume V_{b} is the volume of base-treatment solution that has been added to the HCl-acidified canine blood. The pH measurement is made using a computer-connected pH meter, and the volume dispense rate has been controlled using a computer-controlled syringe-pump dispenser. The near-isotonic base-treatment solutions containing strong bases Na₂CO₃ and NaOH/NaCl have nearly identical titration curves for small volumes. V_{b}≦1 mL, and these solutions raise pH into a desirable range from about 7.3 to about 7.4 at much smaller V_{b} than the base-treatment solution of the weak base NaHCO₃.

### Designing Optimal Base-Treatment Solutions Based on Measurements

Our measurements of blood chemistry and visual appearance of canine blood after the addition of a variety of different base-treatment solutions point to near-isotonic solutions of strong bases as being close to optimal for treating certain forms of acidosis, including acute metabolic acidosis.

In particular, near-isotonic solutions containing strong bases Na₂CO₃ and NaOH can be used to raise pH, while lowering PCO2 without causing substantial visible damage to RBCs.

Although NaHCO₃ can be used as a component in base-treatment solutions, it is not optimal for use on its own, unless a significant increase in PCO2 is desired in while slightly raising pH. Moreover, we find that for base-treatment solutions that are mixtures of NaHCO₃ and Na₂CO₃, the most effective mixtures for raising pH and lowering PCO2 are those containing significantly more Na₂CO₃ than NaHCO₃. Carbicarb as originally formulated, has a total ion concentration that is far above isotonic, and as a result, visible damage to a large fraction of RBCs is observed. Thus, carbicarb is not optimal as a base-treatment solution, at least compared to others we have tested so far.

Administration of any base-treatment solution at a concentration far above or far below isotonic should be avoided, except perhaps in extreme circumstances, at least from the standpoint of visible damage to RBCs, which can be significant.

A gradual administration of a near-isotonic solution of a strong base or mixture of bases is effective in raising pH, lowering PCO2, and minimizing visible damage to RBCs in base-treated blood. This combination of features is highly desirable for the treatment of acidosis. This represents a non-obvious improvement over the prior art in several aspects: (1) we have identified near-isotonic base-treatment solutions as being preferable to highly concentrated base-treatment solutions in minimizing visible damage to RBCs, (2) we have identified that near-isotonic solutions of strong bases, such as those containing Na₂CO₃ and NaOH, are effective in raising pH, lowering PCO2, and enabling tuning of the amount of reduction of PCO2 and increase in [HCO₃⁻], all while minimizing visible damage to RBCs, and (3) volumes of near-isotonic base-treatment solution of strong bases are required to treat acidosis in a human are reasonable and not extraordinarily large because strong bases, such as Na₂CO₃ and NaOH, are much more effective in raising pH than weak bases, such as NaHCO₃.

In addition, if desired, the concentration of strong base in a base-treatment solution can be lowered below near-isotonic conditions, and a salt that is not active in acid-base chemistry, such as NaCl can be added to the base-treatment solution to keep its total concentration at a near-isotonic condition.

We also find that other non-carbonate near-isotonic base-treatment solutions, such as Tris solution whether or not used in combination with saline, can effectively raise pH and lower PCO2 when used to treat blood, but such non-carbonate base-treatment solutions do not tend to significantly raise (i.e. replenish) the bicarbonate ion concentration in the treated blood. Thus, a combination of carbonate and non-carbonate base-treatment solutions that have a total concentration that is near-isotonic can be used to control and adjust to a desired level the increase in pH, decrease in PCO2, and change in bicarbonate ion concentration. Optionally, salts such as NaCl can be added to the base-treatment solution to increase the concentration of osmolites in order to make such mixed base-treatment solutions near-isotonic if the total concentration of osmolites is substantially below the near-isotonic range.

Furthermore, it can be desirable to administer a near-isotonic base-treatment solution containing a strong base to a patient using a computer-controlled dispenser that can adjust the relative concentrations of base and/or saline species in a base-treatment solution. The volume of base-treatment solution administered, volume rate of administration of the base-treatment solution, relative concentration of species in the base-treatment solution (i.e. composition of each type of species), can all be adjusted by the computer-controlled administration system. In addition, measurements of blood pH, PCO2, and other blood parameters, can be sent to the control computer from measurement devices, such as pH meters and blood gas analyzers, and, via a feedback loop, this real-time measured information about a patient's blood can be used by the control computer to adjust the composition, concentration, rate of administration, and total volume administered of a base-treatment solution to a patient.

### Examples of Human Treatment Based on Measured Values Using Near-Isotonic Strong Base-Treatment Solutions

Here, we estimate the volume of isotonic or near-isotonic strong base-treatment solution Vₜ of mixtures of Na₂CO₃ and NaOH at 150 mM total strong base concentration required to treat V_{b}=4.75 L of human blood (a rough estimate of the total blood volume of an adult human) and raise the blood pH by ΔpH=+0.3 pH units. Based on the measured average slope of χ≈0.004 pH unit increase per mM of strong base solution added at 1 part to 9 parts blood, we find that about Vₜ=0.25 L of administered strong base-treatment solution at Cₜ=150 mM will be required. This effectively corresponds to 1 part 150 mM strong base-treatment solution to 19 parts blood, a different ratio than what was used in the experiments, yet achieves the desired pH increase. The formula corresponding to this scenario is:

$$\begin{matrix}Δ pH = 10 [ {V}_{t} / ( {V}_{t} + {V}_{b} ) ] χ {C}_{t} \\ = 10 \times [ 0.25 L / ( 0.25 L + 4.75 L ) ] \times 0.004 pH unit \text{/} mM \times 150 mM \\ = 0.3 pH unit .\end{matrix}$$

Alternatively, if one desires a ratio of 1 part base-treatment solution to 9 part blood ratio and yet one still wants to obtain a ΔpH=0.30 pH units, one can simply lower the strong base-treatment solution molarity to Cₜ=75 mM and boost the ionic strength of the strong base-treatment solution by adding NaCl at 75 mM to keep the total ionic strength at or near an isotonic condition. Here, we take the blood volume to be V_{b}=4.5 L and the volume of strong base-treatment solution to be Vₜ=0.5 L. In this scenario, the same formula applies:

$$\begin{matrix}Δ pH = 10 [ {V}_{t} / ( {V}_{t} + {V}_{b} ) ] χ {C}_{t} \\ = 10 \times [ 0.5 L / ( 0.5 L + 4.5 L ) ] \times 0.004 pH unit \text{/} mM \times 75 mM \\ = 0.3 pH unit\end{matrix}$$

Given the significantly larger total liquid volume compared to the blood volume in an adult human, the pH change in an actual treatment scenario of a live patient would be less than what is calculated above, but this larger total liquid volume could be substituted in the formula above in order to estimate the volume and/or concentration of a strong base-treatment solution needed to raise blood pH a desired amount. Given the above estimates, it is likely that one to two liters of near-isotonic strong base-treatment solution could effectively raise pH while lowering PCO2 without significantly damaging red blood cells.

### Difference Between Tris Solution and Tris Buffer

There is a significant difference between Tris solution and Tris buffer. This difference is typically not clearly explained in the literature. Tris solution is a solution of the base Tris, which is a proton acceptor and reacts with water to produce hydroxide, thereby raising pH. As shown in Table VII, the measured pH of a 150 mM Tris solution (containing some saline that does not participate in acid-base equilibria) is about 10.0. This pH of Tris solution is much higher than the pH associated with 150 mM Tris buffer, measured to be 8.1. Tris buffer can be made from Tris solution by adding an adequate quantity of HCl to a Tris solution until a pH of 8.1 is reached. Thus, Tris solution is different than Tris buffer, and Tris solution has a greater capacity to raise blood pH than Tris buffer if both the Tris solution and the Tris buffer are at the same molarity.

Delivery of Optimized Treatment Solutions Using Computer-Controlled Liquid Dispensing Pumps with or without Computer Feedback Control for Treating Patients Who have Blood- and Body-Chemistry Disorders Including but not Limited to Acidosis

We have developed a computer-controlled multi-liquid dispensing system that is capable of delivering and changing in real-time the composition and concentration of delivered base-treatment solutions using feedback from real-time measurements of blood parameters, including but not limited to pH.

As an example embodiment, we have built a computer-controlled, multi-liquid, multi-injection-point dispensing system using a dual syringe pump computer-controllable liquid dispenser, a pH meter that is equipped to send measurements to a computer, and a control computer programmed with software that reads electronic signals from the pH meter through a first conducting cable and sends commands to the liquid dispenser electronically through a second conducting cable. The use of the pH meter is optional, but this pH meter can provide information about an important blood parameter, pH, that can be used to change the composition, concentration, rate of delivery, and total fluid delivered in real-time through a feedback loop as programmed in the control computer's software. Alternatively, other ion sensitive electrodes, not just pH electrodes, can be used with the pH meter. A computer-connected UV-Vis spectrometer can be employed as part of the apparatus for measuring blood-gas parameters such as PCO2 and PO2 that can be used in choosing the types of liquids, compositions of liquids, volume rates of delivery of liquids, and total volume of liquids delivered.

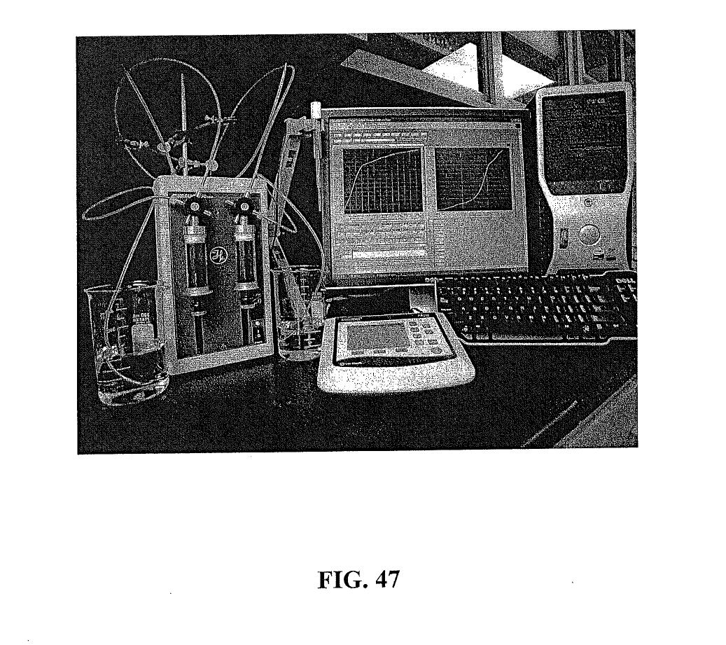

A Hamilton dual syringe pump system (MicroLab 560), containing two computer-controlled motors to drive the dispensing of the syringes, two syringes, and two computer-controlled values, is digitally linked to a Dell computer (Precision 490) via the first of its two built-in serial communications ports. An Accumet pH meter (model AB150) with a digital output is equipped with a pH electrode (Thermo Scientific Orion micro pH), and this pH meter is also digitally linked to the Dell computer using the second of its two built-in serial communications ports. LabVIEW programming environment by National Instruments is loaded onto the Dell computer and a software program (i.e. LABVIEW virtual instrument) has been written in the LabVIEW programming environment to control the dispensing of the treatment liquids by the Hamilton syringe pump system, to read signals from the Accumet pH meter, and to display and record the dispensed liquids and pH measurement in real-time. The two syringes are loaded with two different base-treatment solutions and the output of the dual syringe pump system is typically the combined outflow from the two syringes (i.e. the liquids dispensed by each of the two syringes are typically combined using a Y-type coupler for the tubing outputs of each syringe). Alternatively, the liquids dispensed by each of the two syringes are not combined, but instead are directed to different injection points into the patient's circulatory system. If necessary, the Hamilton dual syringe pump automatically reloads the syringes from large liquid reservoirs of the dispensed liquids using two separate built-in computer-controlled valves that are located at the ends of the two syringes. Thus, the total volume dispensed is not limited to the syringe volume, which is typically between about 10 mL up to 50 mL. An image of the computer-controlled liquid-treatment system that we have created is shown in FIG. 47.

Other common types of computer-controlled liquid dispensing pumps, such as peristaltic, diaphragm, and progressing cavity pumps, could alternatively be used in combination with reservoirs of liquids to be dispensed.

We have written a computer program using LabVIEW that independently controls the dispense rates of two different liquids (e.g. such as base-treatment solutions and/or saline solutions), total amounts of the two different dispensed liquids that can be injected into a patient's blood vessels at one or more injection points. These rates and amounts are adjusted in real-time using feedback of the pH measurement from the pH meter; this pH measurement can be made real-time on the patient's blood, sampled at a location in the patient's circulatory system that is different than any of the injection points so that the liquids have been adequately mixed with the patient's blood. A particular patient's information (e.g. such as weight, height, sex, age, medical condition, blood-gas parameters, and/or genetic information) is entered into the software of the control computer, and the software in the control computer uses this information to customize the types of liquids, compositions of liquids, volume rates of delivery of liquids, and total volume of liquids delivered to a particular patient, based on a database of treatment parameters and equations related to optimal treatment as programmed into the software of the control computer. The liquid-handling components of the apparatus can be sterilized for repeated use.

In another embodiment, based on stored information about the efficacy of base-treatment solutions, stored equations related to how different base-treatment solutions provide different changes in pH and PCO2, information entered about a patient, and real-time information provided by input devices such as a pH meter, the software recommends to a physician the types, compositions, volume rates, and total volumes of liquids to be administered to the patient in order to treat that patient's specific symptoms of acidosis. The physician can either approve the software-recommended treatment or the physician can manually override this and enter desired treatment solution types, compositions, concentrations, volume-dispense rates, and total liquid volumes dispensed.

TABLE I
Responses of canine blood of 9 May 2013 to added near-isotonic solutions
(1 part solution added to 9 parts canine blood 9 May 2013)

| | | | PCO₂ | | | | |
|---|---|---|---|---|---|---|---|
| Added | Conc. | | (mm | [HCO₃⁻] | [Na⁺] | [K⁺] | [Cl⁻] |
| solution | (mM) | pH | Hg) | (mM) | (mM) | (mM) | (mM) |
| none | 0 | 6.90 | 87 | 15.8 | 148 | 4.2 | 98 |
| Na₂CO₃ | 150 | 7.57 | 43 | 37.1 | 164 | 3.4 | 103 |
| NaOH | 150 | 7.47 | 23 | 15.3 | 147 | 3.6 | 98 |
| Na₂CO₃:NaOH | 75:75 = | 7.47 | 36 | 24.0 | 156 | 3.5 | 100 |
| (1:1) | 150ₜₒₜ | | | | | | |
| HCl | 150 | 6.62 | 113 | 10.8 | 128 | 3.8 | 94 |

TABLE II
Responses of HCl-acidified canine blood of 9 May 2013 to added
near-isotonic base solutions (1 part solution added to 9 parts
HCl-acidified canine blood).

| Added | | | PCO₂ | | | | |
|---|---|---|---|---|---|---|---|
| base | Conc. | | (mm | [HCO₃⁻] | [Na⁺] | [K⁺] | [Cl⁻] |
| solution | (mM) | pH | Hg) | (mM) | (mM) | (mM) | (mM) |
| none | 0 | 6.62 | 113 | 10.8 | 128 | 3.8 | 94 |
| NaHCO₃ | 150 | 6.82 | 141 | 21.2 | 135 | 3.1 | 92 |
| Na₂CO₃ | 150 | 7.31 | 61 | 28.4 | 153 | 3.0 | 98 |
| NaOH | 150 | 7.21 | 23 | 8.3 | 135 | 3.2 | 95 |
| Na₂CO₃:NaOH | 75:75 = | 7.37 | 28 | 15.3 | 143 | 3.1 | 97 |
| (1:1) | 150ₜₒₜ | | | | | | |

TABLE III
Responses of HCl-acidified canine blood of 9 May 2013 to added hypertonic
base solutions (1 part solution added to 9 parts HCl-acidified canine blood).

| Added | | | | | | | |
|---|---|---|---|---|---|---|---|
| base | Conc. | | PCO₂ | [HCO₃⁻] | | [K⁺] | [Cl⁻] |
| solution | (mM) | pH | (mm Hg) | (mM) | [Na⁺] (mM) | (mM) | (mM) |
| none | 0 | 6.62 | 113 | 10.8 | 128 | 3.8 | 94 |
| NaHCO₃:Na₂CO₃ | 167:167 | 7.76 | 38 | 50.5 | 171 | 2.7 | 106 |
| NaHCO₃:Na₂CO₃ | 333:333 | 8.36 | 13 | | | 2.6 | 118 |

pH value in italics represents a non-BGA solution probe measurement.

TABLE IV
Responses of canine blood 7 May 2013 to added base-treatment solutions of sodium
bicarbonate and disodium carbonate. The total carbonate species concentration
of the added base-treatment solution is fixed at 150 mM.
(1 part base-treatment solution added to 9 parts canine blood 7 May 2013).

| Added | | | | | | | |
|---|---|---|---|---|---|---|---|
| base | Conc. | | PCO₂ | [HCO₃⁻] | [Na⁺] | [K⁺] | [Cl⁻] |
| solution | (mM) | pH | (mm Hg) | (mM) | (mM) | (mM) | (mM) |
| none | 0 | 6.98 | 75 | 16.4 | 148 | 3.5 | 98 |
| NaHCO₃:Na₂CO₃ | 0:150 | 7.54 | 44 | 34.7 | 162 | 2.8 | 103 |
| NaHCO₃:Na₂CO₃ | 25:125 | 7.50 | 47 | 34.3 | 162 | 2.8 | 102 |
| NaHCO₃:Na₂CO₃ | 37.5:112.5 | 7.54 | 47 | 36.9 | 162 | 2.8 | 101 |
| NaHCO₃:Na₂CO₃ | 75:75 | 7.41 | 57 | 33.6 | 158 | 2.9 | 99 |

TABLE V
Responses of canine blood of 21 May 2013 to added near-isotonic
solutions (1 part solution added to 9 parts canine blood 21 May 2013)

| | | | PCO₂ | | | | |
|---|---|---|---|---|---|---|---|
| Added | Conc. | | (mm | [HCO₃⁻] | [Na⁺] | [K⁺] | [Cl⁻] |
| solution | (mM) | pH | Hg) | (mM) | (mM) | (mM) | (mM) |
| none | 0 | 7.00 | 73 | 16.6 | 150 | 3.3 | 101 |
| Na₂CO₃ | 150 | 7.57 | 43 | 37.1 | 164 | 3.4 | 103 |
| NaOH | 150 | 7.57 | 20 | 17.1 | 146 | 2.9 | 99 |
| NaOH:NaCl | 150:75 | 7.54 | 21 | 16.4 | 155 | 2.9 | 105 |
| Tris Soln | 150 | 7.50 | 27 | 19.5 | 135 | 2.9 | 98 |
| Tris Soln:NaCl | 150:75 | 7.50 | 26 | 19.1 | 144 | 2.9 | 104 |
| Tris Buffer | 150 | 7.18 | 41 | 14.4 | 134 | 3.0 | 100 |
| HCl | 150 | 6.66 | 115 | 11.9 | 130 | 3.0 | 95 |

TABLE VI
Responses of canine blood of HCl-acidified 21 May 2013 to added near-isotonic
solutions (1 part solution added to 9 parts HCl-acidified canine blood 21 May 2013)

| Added | Conc. | | PCO₂ | [HCO₃⁻] | [Na⁺] | [K⁺] | [Cl⁻] |
|---|---|---|---|---|---|---|---|
| solution | (mM) | pH | (mm Hg) | (mM) | (mM) | (mM) | (mM) |
| none | 0 | 6.66 | 115 | 11.9 | 130 | 3.0 | 95 |
| NaOH:NaCl | 150:75 | 7.33 | 21 | 10.1 | 143 | 2.6 | 100 |
| Tris Soln:NaCl | 150:75 | 7.24 | 28 | 11.0 | 131 | 2.6 | 100 |
| Tris Buffer | 150 | 6.96 | 42 | 8.8 | 122 | 2.7 | 98 |
| Na₂CO₃ | 150 | 7.39 | 47 | 26.2 | 152 | 2.5 | 99 |
| NaHCO₃:Na₂CO₃ | 25:125 | 7.31 | 60 | 27.4 | 152 | 3.6 | 99 |
| NaHCO₃:Na₂CO₃ | 37.5:112.5 | 7.20 | 73 | 26.4 | 150 | 4.3 | 99 |
| NaHCO₃:Na₂CO₃ | 75:75 | 7.04 | 101 | 25.2 | 145 | 5.6 | 98 |
| NaHCO₃ | 150 | 6.86 | 135 | 22.6 | 136 | 2.6 | 93 |

TABLE VII
Measured pH of base-treatment solutions. Measurements are
made using an Accumet pH meter equipped with a calibrated
pH electrode.

| | Measured |
|---|---|
| Base-Treatment Solution | pH |
| 150 mM NaHCO₃ | 8.18 |
| 1.0M NaHCO₃ | 8.10 |
| 75 mM Na₂CO₃ | 11.18 |
| 100 mM Na₂CO₃ | 11.33 |
| 150 mM Na₂CO₃ | 11.42 |
| 200 mM Na₂CO₃ | 11.35 |
| 250 mM Na₂CO₃ | 11.35 |
| 360 mM Na₂CO₃ | 11.37 |
| 1.0M Na₂CO₃ | 11.66 |
| 100 mM NaOH:100 mM NaCl | 12.91 |
| 150 mM NaOH | 12.95 |
| 75 mM Na₂CO₃:75 mM NaOH | 12.64 |
| 150 mM Tris:75 mM NaCl | 10.08 |
| 150 mM Tris Buffer | 8.11 |
| 167 mM NaHCO₃:167 mM Na₂CO₃ | 9.80 |
| 333 mM NaHCO₃:333 mM Na₂CO₃ | 9.63 |
| 25 mM NaHCO₃:125 mM Na₂CO₃ | 10.48 |
| 37.5 mM NaHCO₃:112.5 mM Na₂CO₃ | 10.23 |

### First Example Embodiment of Treating an Acidotic Rat Using a Near-Isotonic Base-Treatment Solution Containing a Strong Base

As a first example embodiment, we treat a 246.6 g male Sprague-Dawley (SD) rat, hereafter referred to as RAT A. RAT A is intubated using an oxygen ventilator, set at about 53 breaths/minute using 1.5% isoflurane as an anesthetic agent, according to an approved procedure 1999-028 (UCLA Medical School Physiology). This form of anesthesia permits good heart, lung, liver, and kidney function in an intubated rat. RAT A has catheters inserted into the jugular, a femoral vein, and a femoral artery. The blood is heparinized in order to prevent clotting of blood in the catheters. We first induce an acidotic state, characterized by lower than normal levels of pH and bicarbonate ion concentration [HCO₃⁻], by bleeding RAT A and then injecting a 0.55 M lactic acid solution intravenously into RAT A via the femoral vein. Blood-gas parameters (pH, PCO2, and [HCO₃-]) as well as blood ion concentrations ([Na+], [Cl−], and [K+]) are measured on 200 microliter blood samples taken from RAT A using an IDEXX VetStat Blood-Gas Analyzer (BGA) with disposable Electrolyte 8+ cassettes throughout the experiment. After establishing an acidotic state, we then treat RAT A by injecting a near-isotonic base solution containing a strong base intravenously. This base solution contains 112.5 mM disodium carbonate (Na₂CO₃, a strong base) and 37.5 mM sodium bicarbonate (NaHCO₃, a weak base) in water.

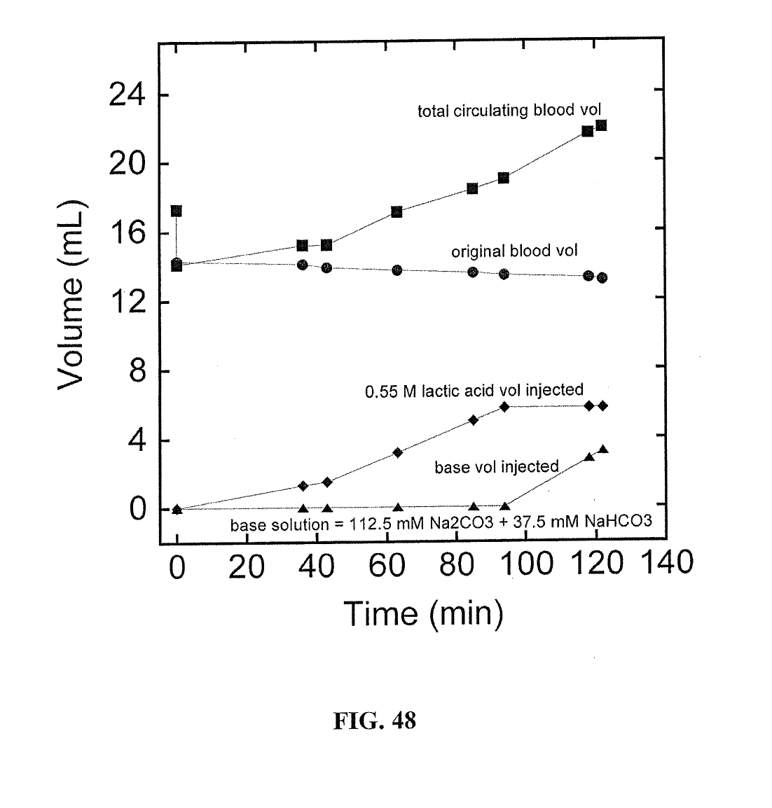

We estimate the original circulating blood volume in milliliters as being about 7% of the rat's body weight in grams. The total circulating blood volume (including all injected solutions) and the remaining original blood volume are plotted as a function of time in FIG. 48. The initial step-reduction in both reflects the initial bleed of the rat, which is used as the reference point of time=0 min. Thereafter, the total circulating blood volume increases because of the injected acid and base solutions, whereas the remaining original blood volume decreases slightly because of dilution by the injected acid and base solutions. The volumes shown in FIG. 1 account for the small volume of blood removed for each of the BGA measurements. After the initial bleed, 0.55 M lactic acid solution is injected; the volume of acid solution injected is also shown in the lower part of FIG. 48. Once the BGA parameters have reached a sufficiently strong acidotic state, corresponding to a total injected volume of the acid solution of about 5.7 mL, we stop the injection of acid solution. Thereafter, we administer 112.5 mM Na₂CO₃+37.5 mM NaHCO₃ base solution that has a near-isotonic osmolite concentration by injection into the femoral vein using a programmable automated syringe pump; the volume of injected base solution as a function of time is shown in the lower part of FIG. 48. The total volume of injected base at the end of the injection is about 3.3 mL.

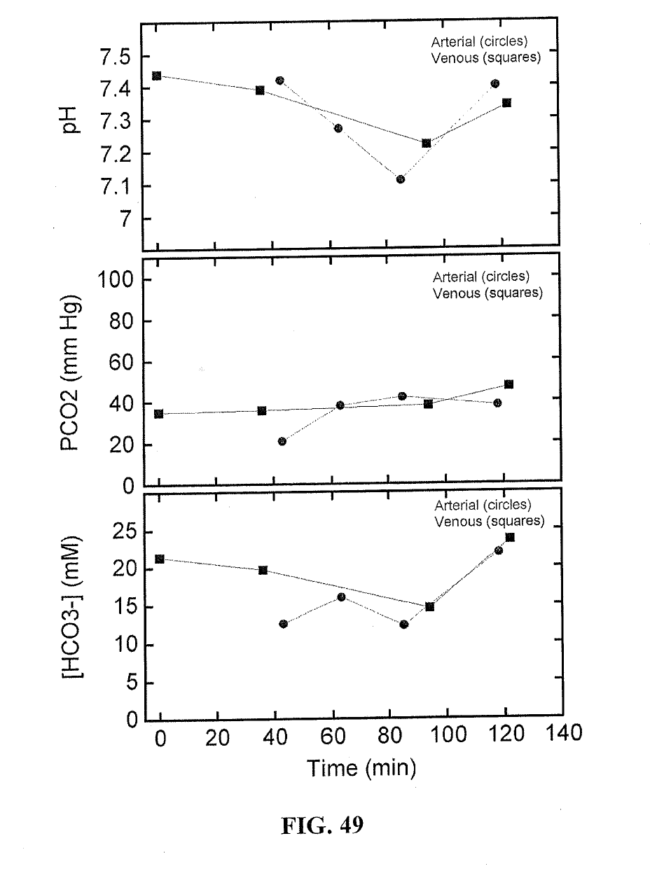

We show the dependence of the BGA parameters as a function of time for RAT A in FIG. 49. Initially at time=0, prior to injecting the acid solution, for arterial blood, the pH is pH=7.44, the carbon dioxide pressure is PCO2=35 mm Hg, and the bicarbonate ion concentration is [HCO₃⁻]=21.4 mM. These values are in the normal range for an SD rat. After the initial bleed and injection of 5.7 mL of 0.55 M lactic acid solution, we measure the following values for BGA parameters: pH=7.11 (arterial) and pH=7.22 (venous); PCO2=42 mm Hg (arterial) and PCO2=38 mm Hg (venous); and [HCO₃⁻]=12.3 mM (arterial) and [HCO₃⁻]=14.5 mM (venous), as shown around 80 min<Time<90 min in FIG. 49. The reductions in pH and [HCO₃⁻] below the normal range clearly indicate that the injected acid solution has created an acidotic state in RAT A. Following this, we treat the acidotic RAT A using base solution. After treatment with 3.3 mL of injected base solution, we measure the following for the BGA parameters of RAT A: pH=7.40 (arterial) and pH=7.34 (venous); PCO2=38 mm Hg (arterial) and PCO2=47 mm Hg (venous); and [HCO₃⁻]=21.9 mM (arterial) and [HCO₃⁻]=23.6 mM (venous), as shown at the longest time in FIG. 2. These values for the BGA parameters are in the normal range, so the near-isotonic base treatment solution has efficiently treated the acidotic state that had previously been induced in RAT A.

Treatment of the acidotic state of RAT A by about 3.3 mL of the near-isotonic base solution (112.5 mM Na₂CO₃+37.5 mM NaHCO₃) results in: an increase in pH (averaged over arterial and venous), ΔpH=+0.26; a very small average change in pressure of CO₂, ΔPCO2=+2.5 mm Hg; and an average increase in bicarbonate ion concentration, Δ[HCO₃⁻]=+9.4 mM. Thus, as predicted by a theoretical model, use of predominantly disodium carbonate, rather than sodium bicarbonate, in the base-treatment solution has caused both pH and [HCO₃⁻] to increase substantially without causing a large increase in PCO2. This is a highly desirable result.

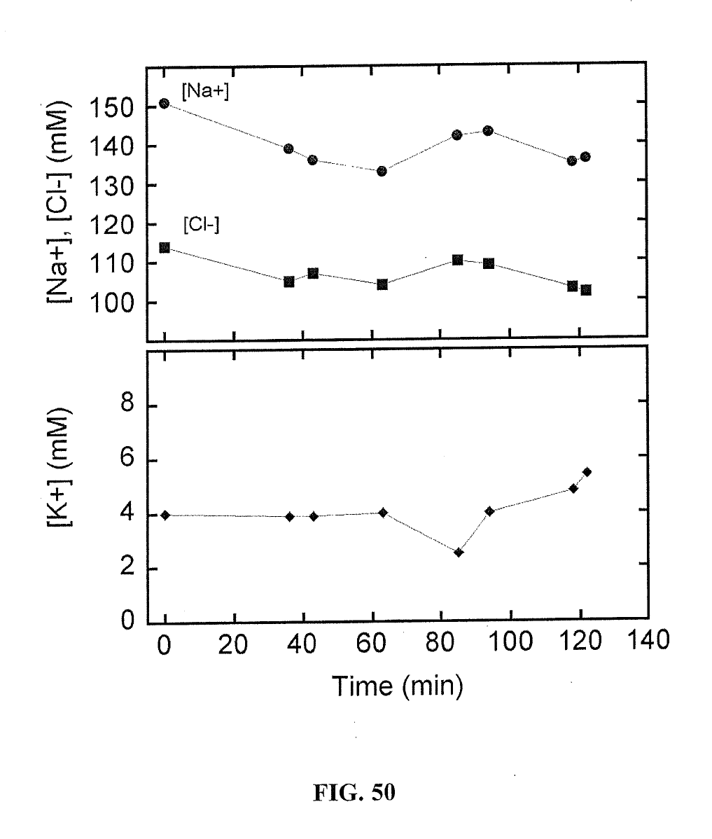

In addition to tracking BGA parameters, we also have measured [Na⁺], [Cl⁻], and [K⁺] ion concentrations in the blood of RAT A as a function of time during the experiment, as shown in FIG. 50. Slight decreases are seen in [Na⁺] and [Cl⁻], and [K⁺] remains essentially unchanged.

RAT A survived until the end of the experiment and could have survived significantly longer, but the approved protocol required sacrificing the animal while it was under anesthetic. Heart and lungs appeared to be in good condition after treatment with the base solution.

From the data we have obtained from RAT A, we estimate changes in average pH and average [HCO₃⁻] per mL of near-isotonic base solution injected per gram of body weight of RAT A. Thus, for this particular base-treatment solution, we can estimate for the pH change: +0.26/(3.3 mL/246.6 g)=+19.4 kg/L, and we would estimate for the bicarbonate ion change: +9.4 mM/(3.3 mL/246.6 g)=+0.70 M (kg/L).

Extrapolating these results from the rat scale to the human scale, to obtain a +0.2 increase in blood pH in a human weighing 60 kg, the relevant equation is +0.2=+19.4 kg/L×(V_{base}/60 kg), so V_{base}=0.62 L of base-treatment solution would have to be administered to the human. This is less volume than is typically required using sodium bicarbonate solution, thereby indicating that the base-treatment solution we used to treat RAT A, which contains a large proportion of strong base, is more efficacious in treating an acidotic state than a sodium bicarbonate solution. With V_{base}=0.62 L of the same base-treatment solution administered to the human, we would estimate a corresponding increase in bicarbonate ion concentration in the human of +7.2 mM.

### Second Example Embodiment of Treating an Acidotic Rat Using a Near-Isotonic Base-Treatment Solution Containing a Strong Base

As a second example embodiment, we induce a state of acidemia in a rat using a different approach, gavage of an aqueous solution of the acid ammonium chloride (NH₄Cl), and then we treat the induced acidotic rat using a near-isotonic base-treatment solution that is dominantly composed of a strong base. For this second embodiment, the rat is a male SD weighing 340.8 g (date of birth May 26, 2013), hereafter referred to as RAT B. We fast the rat (food only) for 12 hours and then measure BGA and ion parameters using the IDEXX VetStat from RAT B's orbital blood: pH=7.39, PCO2=51 mm Hg, [HCO₃⁻]=28.8 mM, [Na+]=142 mM, [Cl—]=106 mM, and [K+]=4.3 mM. We then administer a first gavage of 3.5 mL of a 2.5% w/v NH₄Cl solution to RAT B. After waiting 5 minutes for the solution to be taken up, we administer a second gavage of 3.5 mL of a 2.5% w/v NH₄Cl solution to RAT B.

Roughly one hour after the second gavage, RAT B is intubated using an oxygen ventilator, set at about 53 breaths/minute using 1.5% isoflurane as an anesthetic agent, according to an approved procedure 1999-028 (UCLA Medical School Physiology). This form of anesthesia permits good heart, lung, liver, and kidney function in an intubated rat. We insert catheters into RAT B in the following blood vessels: jugular, a femoral vein, and a femoral artery. The blood is heparinized in order to prevent clotting of blood in the catheters. Roughly 2 hours and 50 minutes after the second gavage, the following BGA and ion concentrations of RAT B's blood are measured. Arterial blood has measured values of pH=7.18, PCO2=28 mm Hg, [HCO₃⁻]=9.7 mM, [Na⁺]=137 mM, [Cl⁻]=118 mM, and [K⁺]=5.9 mM. Venous blood has measured values of pH=7.16, PCO2=28 mm Hg, [HCO₃⁻]=9.5 mM, [Na⁺]=140 mM, [Cl⁻]=117 mM, and [K⁺]=6.3 mM. Thus, after gavage, RAT B's pH and bicarbonate ion concentrations are well below the normal range, and are in the range associated with moderate to severe acidosis. An echo experiment, performed using an Acuson echo machine to evaluate cardiac function, reveals an ejection fraction of 49.3%, indicating cardiac function is depressed compared to a normal state in an SD rat.

Roughly 3 hours and 20 minutes after the second gavage, we begin administering a near-isotonic base solution (112.5 mM Na₂CO₃+37.5 mM NaHCO₃) via injection into the femoral vein of RAT B. After 2.5 mL of base solution has been administered, at about 3 hours and 50 minutes after the second gavage, we measure the following BGA parameters and ion concentrations of RAT B. Arterial blood has measured values of pH=7.28, PCO2=29 mm Hg, [HCO₃]=12.6 mM, [Na⁺]=140 mM, [Cl⁻]=117 mM, and [K⁺]=6.2 mM. Venous blood has measured values of pH=7.22, PCO2=33 mm Hg, [HCO₃⁻]=12.6 mM, [Na⁺]=142 mM, [Cl⁻]=117 mM, and [K⁺]=6.1 mM. Thus, there is a measurable increase in pH and [HCO₃-] without much change in PCO2. After removing 2 mL of blood to keep the total blood volume from expanding too much as we inject solution, we continue injecting the same base solution. After an additional 2.5 mL of base treatment solution has been administered, yielding a total of 5.0 mL of base treatment solution, at about 4 hours and 20 minutes after the second gavage, we measure the following BGA parameters and ion concentrations for RAT B. Arterial blood has measured values of pH=7.34, PCO2=31 mm Hg, [HCO₃⁻]=15.6 mM, [Na⁺]=142 mM, [Cl⁻]=115 mM, and [K⁺]=5.2 mM. The increase in pH and [HCO₃⁻] without a significant change in PCO2 are observed as a result of administering the base treatment solution. An echo experiment performed on RAT B to measure cardiac function after base treatment reveals an increase (i.e. improvement) in ejection fraction to 55.5%.

RAT B survived until the end of the experiment and could have survived significantly longer, but the approved protocol required sacrificing the animal while it was under anesthetic. Heart and lungs appeared to be in excellent condition after treatment with the base solution.

From the data we have obtained from RAT B, we estimate changes in average pH and average [HCO₃⁻] per mL of near-isotonic base solution injected per gram of body weight of RAT B. Thus, for this particular base-treatment solution, we can estimate for the pH change: +0.16/(5.0 mL/340.8=+10.9 kg/L, and we would estimate for the bicarbonate ion concentration change: +5.9 mM/(5.0 mL/340.8 g)=+0.40 M (kg/L).

Extrapolating these results from the rat scale to the human scale, based on results for RAT B, to obtain a+0.2 increase in blood pH in a human weighing 60 kg, the relevant equation is +0.2=+10.9 kg/L×(V_{base}/60 kg), so V_{base}=1.10 L of base-treatment solution would have to be administered to the human. This estimate is comparable to or less than the typical volume administered of 1.0 M sodium bicarbonate solution in a typical clinical treatment of acidosis in a human. Thus, the near-isotonic base-treatment solution we used to treat RAT B, which contains a large proportion of strong base Na₂CO₃, can be at least as efficacious in treating an acidotic state than a sodium bicarbonate solution that has a much higher concentration. With V_{base}=1.10 L of the same near-isotonic base-treatment solution administered to the human, we would estimate a corresponding increase in bicarbonate ion concentration in the human of +0.40 M kg/L×1.10 L/60 kg=+7.3 mM.

### Average Values for Predicting Changes in pH and Bicarbonate Ion Concentration

Averaging values for RAT A and RAT B, we estimate that the average pH change for the particular near-isotonic base-treatment solution (112.5 mM Na₂CO₃+37.5 mM NaHCO₃) as being: +15.2 kg/L. Likewise for the change in bicarbonate ion concentration for the same particular near-isotonic base-treatment solution, averaging values for RAT A and RAT B, we estimate: +0.55 M kg/L. For both RAT A and RAT B, the change in PCO2 as a result of administering the near-isotonic base-treatment solution described above is essentially negligible, given the measurement uncertainty of the BGA device.

### Third Example Embodiment of Treating an Acidotic Rat Using a Near-Isotonic Base-Treatment Solution Containing a Strong Base

As a third example embodiment, we induce a state of acidemia in a rat using gavage of an aqueous solution of the acid ammonium chloride (NH₄Cl), and then we treat the induced acidotic rat using an aqueous near-isotonic base-treatment solution containing only disodium carbonate, a strong base. For this third embodiment, the rat is a male SD weighing 379.9 g (date of birth May 26, 2013), hereafter referred to as RAT C. We fast the rat (food only) for 12 hours. We administer a first gavage of 4.0 mL of a 2.5% w/v NH₄Cl solution to RAT C. After waiting 5 minutes for the solution to be taken up, we administer a second gavage of 4.0 mL of a 2.5% w/v NH₄Cl solution to RAT C.

Roughly one hour after the second gavage, RAT C is intubated using an oxygen ventilator, set at about 53 breaths/minute using 1.5% isoflurane as an anesthetic agent, according to an approved procedure 1999-028 (UCLA Medical School Physiology). We insert catheters into RAT C in the following blood vessels: a femoral vein and a femoral artery. The blood is heparinized in order to prevent clotting of blood in the catheters. Roughly 3 hours after the second gavage, the following BGA and ion concentrations of RAT C's arterial blood are measured using the IDEXX VetStat with Electrolyte 8+ cassettes: pH=7.24, PCO2=22 mm Hg, [HCO₃⁻]=8.8 mM, [Na⁺]=142 mM, [Cl⁻]=119 mM, and [K⁺]=4.3 mM. Thus, after gavage, RAT C's pH and bicarbonate ion concentrations are well below the normal range.

Roughly 3 hours and 20 minutes after the second gavage, we begin administering a near-isotonic base solution of disodium carbonate (150 mM Na₂CO₃) via injection at 5 mL/hr into the femoral vein of RAT C. After 2.5 mL of base solution has been administered, at about 3 hours and 50 minutes after the second gavage, we measure the arterial blood of RAT C, yielding the following BGA parameters and ion concentrations: pH=7.38, PCO2=23 mm Hg, [HCO₃⁻]=12.8 mM, [Na⁺]=141 mM, [Cl⁻]=117 mM, and [K⁺]=5.0 mM. Thus, we observe a measurable increase in pH and [HCO₃-] without much change in PCO2. After removing 2 mL of blood to keep the total blood volume from expanding too much as we inject solution, we continue injecting the same base solution. After an additional 2.5 mL of the near-isotonic base treatment solution of 150 mM Na₂CO₃ has been administered, yielding a total of 5.0 mL of base treatment solution (i.e. after an additional 30 minutes of injection of the base solution), we measure the following arterial BGA parameters and ion concentrations for RAT C: pH=7.42, PCO2=24 mm Hg, [HCO₃⁻]=14.4 mM, [Na⁺]=143 mM, [Cl⁻]=118 mM, and [K⁺]=5.0 mM. Thus, RAT C's pH and [HCO₃⁻] have been increased, without causing a significant change in PCO2, as a result of administering the near-isotonic base treatment solution. An echo experiment performed on RAT C to measure cardiac function after base treatment reveals an increase (i.e. improvement) in ejection fraction to 81.8%, which is comparable to and slightly better than RAT C's initial ejection fraction of 74.2% measured by cardiac echo prior to gavage.

Optical transmission microscopy measurements were performed on the blood of RAT C throughout administration of the near-isotonic base-treatment solution, and we did not observe any significant increase in spiculation or other types of damage to RAT C's red blood cells as a result of near-isotonic base administration. RAT C survived until the end of the experiment and could have survived significantly longer, but the approved protocol required sacrificing the animal while it was under anesthetic. Heart and lungs appeared to be in excellent condition after treatment with the strong base solution. No obvious damage to tissue was seen visually near the injection site of the 150 mM Na₂CO₃ base-treatment solution.

From the data we have obtained from RAT C, we estimate the changes in average pH and average [HCO₃⁻] per mL of near-isotonic base solution of pure disodium carbonate (150 mM Na₂CO₃) injected per gram of body weight of RAT C. We estimate the pH change to be: +0.18/(5.0 mL/379.9 g)=+13.7 g/mL=+13.7 kg/L, and we estimate for the bicarbonate ion concentration change: +5.6 mM/(5.0 mL/379.9 g)=+0.43 M (g/mL)=+0.43 M (kg/L).

Extrapolating these results from the rat scale to the human scale, based on results for RAT C, to obtain a+0.2 increase in blood pH in a human weighing 60 kg, the relevant equation is +0.2=+13.7 kg/L×(V_{base}/60 kg), so V_{base}=0.88 L of 150 mM Na₂CO₃ base-treatment solution would have to be administered to the human. Thus, a reasonable volume near 1 L of the base-treatment solution containing only the strong base Na₂CO₃ at a near-isotonic concentration would be efficacious in treating a state of acidemia in a human. Using V_{base}=0.88 L of the same base-treatment solution administered to the human, we would estimate an increase in bicarbonate ion concentration in the human to be: +0.43 M kg/L×0.88 L/60 kg=+6.3 mM.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A system for treating an acidotic patient, comprising:
an intravenous-fluid supply system;
an automated fluid mixer and dispenser connected to said intravenous-fluid supply system to receive at least one supply fluid therefrom;
an electronic control system configured to communicate with said automated fluid mixer and dispenser; and
an intravenous line fluidly connected to said automated fluid mixer and dispenser, said intravenous line comprising an intravenous connecter configured for injecting intravenous fluid dispensed from said automated fluid mixer and dispenser intravenously into said acidotic patient,
wherein said electronic control system is configured to control at least one of a total volume or a flow rate of said intravenous fluid to be injected into said acidotic patient's blood based on at least a measured pH of said acidotic patient's blood and based on a composition of said at least one supply fluid.

2. A system for treating an acidotic patient according to claim 1, wherein said electronic control system is further configured to control at least one of said total volume or said flow rate of said intravenous fluid to be injected into said acidotic patient's blood based on at least one of a predicted pH effect, a predicted pCO2 effect, and a predicted bicarbonate anion concentration effect of said intravenous fluid to be injected.

3. A system for treating an acidotic patient according to claim 2, wherein said electronic control system is further configured to control at least one of said total volume or said flow rate of said intravenous fluid to be injected into said acidotic patient's blood further based on a predicted effect of said intravenous fluid to be injected on an isotonic condition of said acidotic patient's blood.

4. A system for treating an acidotic patient according to claim 1, wherein said at least one supply fluid from said intravenous-fluid supply system comprises at least a first supply fluid and a second supply fluid, and
wherein said electronic control system is further configured to provide control signals to said automated fluid mixer and dispenser to mix said first and second supply fluids in a proportion based on at least said measured pH of said acidotic patient's blood.

5. A system for treating an acidotic patient according to claim 2, wherein said at least one supply fluid from said intravenous-fluid supply system comprises at least a first supply fluid and a second supply fluid, and
wherein said electronic control system is further configured to provide control signals to said automated fluid mixer and dispenser to mix said first and second supply fluids in a proportion based on predicted pH and pCO2 effects of said intravenous fluid to be injected.

6. A system for treating an acidotic patient according to claim 5, wherein said electronic control system is further configured to provide control signals to said automated fluid mixer and dispenser to mix said first and second supply fluids in a proportion and a concentration based on a predicted effect of said intravenous fluid to be injected on an isotonic condition of said acidotic patient's blood.

7. A system for treating an acidotic patient according to claim 4, wherein said first supply fluid has a higher pH in a base range than a pH of said second solution.

8. A system for treating an acidotic patient according to claim 7, wherein said second supply fluid is an aqueous solution comprising dissolved sodium bicarbonate, and said first solution is an aqueous solution comprising at least one of dissolved disodium carbonate, sodium hydroxide, or tris(hydroxymethyl)aminomethane.

9. A system for treating an acidotic patient according to claim 1, further comprising a blood sensor system configured to communicate with said electronic control system,
wherein said blood sensor system is configured to measure at least one property of said patient's blood in real time and provide sensor signals to said electronic control system, and
wherein said electronic control system provides control signals to said automated fluid mixer and dispenser such that at least one of mixing or dispensing by said automated fluid mixer and dispenser is based at least partially on real-time information from said blood sensor system.

10. A system for treating an acidotic patient according to claim 9, wherein said blood sensor system comprises a pH sensor for real-time blood pH measurements and feedback control of said automated fluid mixer and dispenser by said electronic control system.

11. A system for treating an acidotic patient according to claim 1, further comprising a second intravenous line fluidly connected to said automated fluid mixer and dispenser, said second intravenous line comprising a second intravenous connecter configured for injecting second intravenous fluid dispensed from said automated fluid mixer and dispenser intravenously into a second position in said acidotic patient.

12. A system for treating an acidotic patient according to claim 11, wherein said first mentioned intravenous fluid dispensed from said automated fluid mixer and dispenser is substantially a same composition as said second intravenous fluid dispensed from said automated fluid mixer and dispenser.

13. A system for treating an acidotic patient according to claim 11, wherein said first mentioned intravenous fluid dispensed from said automated fluid mixer and dispenser has a different composition from said second intravenous fluid dispensed from said automated fluid mixer and dispenser, and
wherein said first-mentioned and said second intravenous fluids act together in said acidotic patient's body to adjust at least said pH condition of at least said portion of said acidotic patient's body while maintaining at least a near isotonic condition of said acidotic patient's blood.

14. A system for treating an acidotic patient according to claim 13, wherein said intravenous-fluid supply system comprises a plurality of precursor solutions that when mixed by said automated fluid mixer and dispenser provides an intravenous solution to be dispensed,
wherein said intravenous solution comprises sodium bicarbonate and at least one of disodium carbonate, sodium hydroxide, and tris(hydroxymethyl)aminomethane dissolved in an aqueous solution, and
wherein said intravenous solution has a pH of at least 10 and a total concentration of osmolites within a near isotonic range.

15. A system for treating an acidotic patient according to claim 14, wherein said aqueous solution comprises disodium carbonate and sodium bicarbonate in a molar ratio of at least 2:1.

16. A system for treating an acidotic patient according to claim 15, wherein said intravenous solution consists essentially of said disodium carbonate and sodium bicarbonate dissolved in said aqueous solution.

17. An intravenous solution for treating acidosis, comprising: sodium bicarbonate; and at least one of disodium carbonate, sodium hydroxide, and tris(hydroxymethyl)aminomethane dissolved in an aqueous solution,
wherein said intravenous solution has a pH of at least 10 and a total concentration of osmolites within a near isotonic range.

18. An intravenous solution according to claim 17, wherein said aqueous solution comprises disodium carbonate and sodium bicarbonate in a molar ratio of at least 2:1.

19. An intravenous solution according to claim 18, wherein said intravenous solution consists essentially of said disodium carbonate and sodium bicarbonate dissolved in said aqueous solution.

20. A method of treating acidosis, comprising:
providing an intravenous solution for treating acidosis; and
administering said intravenous solution intravenously to an acidotic patient,
wherein said intravenous solution has a pH of at least 10 and a total concentration of osmolites within a near isotonic range.

21. A method of treating acidosis according to claim 20, wherein said intravenous solution comprises sodium bicarbonate and at least one of disodium carbonate, sodium hydroxide, and tris(hydroxymethyl)aminomethane dissolved in an aqueous solution.

22. A method of treating acidosis according to claim 21, wherein said aqueous solution comprises disodium carbonate and sodium bicarbonate in a molar ratio of at least 2:1.

23. A method of treating acidosis according to claim 23, wherein said intravenous solution consists essentially of said disodium carbonate and sodium bicarbonate dissolved in said aqueous solution.

24. A method of treating acidosis according to claim 20, further comprising selecting said intravenous solution to have a composition based on at least one of a measured pH value, a measured pCO2 value, and a measured bicarbonate anion concentration of said acidotic patient.

25. A method of treating acidosis according to claim 20, further comprising:
measuring a pH of said acidotic patient; and
at least one of selecting or mixing said intravenous solution to have a composition based on said measuring said pH value of said acidotic patient.

26. A method of treating acidosis according to claim 20, further comprising repeating said measuring and at least one of selecting or mixing a plurality of times to provide a real-time adjusted method of treating acidosis.

27. An intravenous dispersion for treating acidosis, comprising:
a liquid; and
a plurality of particles dispersed in said liquid,
wherein each particle of said plurality of particles has a maximum outer dimension of less than about 2 micrometers such that said particles can pass unhindered through capillary blood vessels of an acidotic patient being treated,
wherein each of said plurality of particles comprises at least one of a shell and a matrix material that dissolves at a predetermined rate within said acidotic patient's blood stream, and
wherein each of said plurality of particles comprises a pH-influencing material that mixes in said acidotic patient's blood stream at a controlled rate while said at least one of said shell and said matrix material dissolves.

28. An intravenous dispersion for treating acidosis according to 27, wherein a rate of dissolution of said pH-influencing material is controlled by at least one of a composition of said at least one of said shell and said matrix material, a structure of said at least one of said shell and said matrix material, a relative volume fraction of said pH-influencing material and said at least one of said shell and said matrix material, and an average size of said plurality of particles.
